SEQUENCE LISTING (70 sequences)

SEQ ID NO: 1 - protein, 295 aa, Mus musculus
MSGALDVLQMKEEDVLKLLAAGTHLGGTNLDFQMEQYIYKRKSDGIYIINLKRTWEKLLLAARAIVAIENPADVSVISSRNTGQRAVLKFAAATGATPIAGRFTPGTFTNQIQAAFREPRLLVVTDPRADHQPLTEASYVNLPTIALCNTDSPLAYVDIAIPCNNKGAHSVGLMWWMLAREVLRMRGTISREHPWEVMPDLYFYRDPEEIEKEEQAAAEKAVTKEEFQGEWTAPAPEFTAAQPEVADWSEGVQVPSVPIQQFPTEDWSAQPATEDWSAAPTAQATEWVGATTEWS

SEQ ID NO: 2 - protein, 295 aa, Homo sapiens [sequence omitted]

SEQ ID NO: 3 - protein, 295 aa, Mus musculus
MSGALDVLQMKEEDVLKFLAAGTHLGGTNLDFQMEQYIYKRKSDGIYIINLKRTWEKLLLAARAIVAIENPADVSVISSRNTGQRAVLKFAAATGATPIAGRFTPGTFTNQIQAAFREPRLLVVTDPRADHQPLTEASYVNLPTIALCNTDSPLRYVDIAIPCNNKGAHSVGLMWWMLAREVLRMRGTISREHPWEVMPDLYFYRDPEEIEKEEQAAAEKAVTKEEFQGEWTAPAPEFTAAQPEVADWSEGVQVPSVPIQQFPTEDWSAQPATEDWSAAPTAQATEWVGATTEWS

SEQ ID NO: 4 - protein, 16 aa, Artificial Sequence
  Description of Artificial Sequence Synthetic peptide
AAEKAVTKEEFQGEWT

SEQ ID NO: 5 - protein, 12 aa, Artificial Sequence
  Description of Artificial Sequence Synthetic peptide
QFPTEDWSAQPA

SEQ ID NO: 6 - protein, 16 aa, Artificial Sequence
  Description of Artificial Sequence Synthetic peptide
QFPTEDWSAQPATEDW

SEQ ID NO: 7 - protein, 10 aa, Artificial Sequence
  Description of Artificial Sequence Synthetic peptide
GYTFTSYNMH

SEQ ID NO: 8 - protein, 17 aa, Artificial Sequence
  Description of Artificial Sequence Synthetic peptide
YIYPGNGGTNYNQKFKG

SEQ ID NO: 9 - protein, 15 aa, Artificial Sequence
  Description of Artificial Sequence Synthetic peptide
GGYYYGSSWELYFDY

SEQ ID NO: 10 - protein, 16 aa, Artificial Sequence
  Description of Artificial Sequence Synthetic peptide
RSSQSIVHSNGNTYLE

SEQ ID NO: 11 - protein, 7 aa, Artificial Sequence
  Description of Artificial Sequence Synthetic peptide
KVSNRFS

SEQ ID NO: 12 - protein, 9 aa, Artificial Sequence
  Description of Artificial Sequence Synthetic peptide
FQGSHVPPT

SEQ ID NO: 13 - protein, 10 aa, Artificial Sequence
  Description of Artificial Sequence Synthetic peptide
GFSLTAYGVN

SEQ ID NO: 14 - protein, 16 aa, Artificial Sequence
  Description of Artificial Sequence Synthetic peptide
MIWGNGDTDYNSALKS

SEQ ID NO: 15 - protein, 3 aa, Artificial Sequence
  Description of Artificial Sequence Synthetic peptide
YGY

SEQ ID NO: 16 - protein, 16 aa, Artificial Sequence
  Description of Artificial Sequence Synthetic peptide
KSSQSLLDSDGKTYLN

SEQ ID NO: 17 - protein, 7 aa, Artificial Sequence
  Description of Artificial Sequence Synthetic peptide
LVSKVDS

SEQ ID NO: 18 - protein, 9 aa, Artificial Sequence
  Description of Artificial Sequence Synthetic peptide
WQGTHFPFT

SEQ ID NO: 19 - protein, 10 aa, Artificial Sequence
  Description of Artificial Sequence Synthetic peptide
GFTFSSYTMS

SEQ ID NO: 20 - protein, 17 aa, Artificial Sequence
  Description of Artificial Sequence Synthetic peptide
TISSGGTYTYYPDSVKG

SEQ ID NO: 21 - protein, 3 aa, Artificial Sequence
  Description of Artificial Sequence Synthetic peptide
LRY

SEQ ID NO: 22 - protein, 16 aa, Artificial Sequence
  Description of Artificial Sequence Synthetic peptide
KSGQSLLDSDGKTYLN

SEQ ID NO: 23 - protein, 7 aa, Artificial Sequence
  Description of Artificial Sequence Synthetic peptide
LVSKLDS

SEQ ID NO: 24 - protein, 9 aa, Artificial Sequence
  Description of Artificial Sequence Synthetic peptide
WQGTHFPQT

SEQ ID NO: 25 - protein, 10 aa, Artificial Sequence
  Description of Artificial Sequence Synthetic peptide
GFSLTSYDIS

SEQ ID NO: 26 - protein, 16 aa, Artificial Sequence
  Description of Artificial Sequence Synthetic peptide
VIWTGGGTNYNSAFMS

SEQ ID NO: 27 - protein, 4 aa, Artificial Sequence
  Description of Artificial Sequence Synthetic peptide
SFVY

SEQ ID NO: 28 - protein, 16 aa, Artificial Sequence
  Description of Artificial Sequence Synthetic peptide
RSSQSLVHSNGNTYLH

SEQ ID NO: 29 - protein, 9 aa, Artificial Sequence
  Description of Artificial Sequence Synthetic peptide
SQSTHVPWT

SEQ ID NO: 30 - protein, 6 aa, Artificial Sequence
  Description of Artificial Sequence Synthetic 6xHis tag
HHHHHH

SEQ ID NO: 31 - protein, 12 aa, Artificial Sequence
  Description of Artificial Sequence Synthetic peptide
MLAREVLRMRGT

SEQ ID NO: 32 - protein, 12 aa, Artificial Sequence
  Description of Artificial Sequence Synthetic peptide
EVLRMRGTISRE

SEQ ID NO: 33 - protein, 12 aa, Artificial Sequence
  Description of Artificial Sequence Synthetic peptide
MRGTISREHPWE

SEQ ID NO: 34 - protein, 12 aa, Artificial Sequence
  Description of Artificial Sequence Synthetic peptide
ISREHPWEVMPD

SEQ ID NO: 35 - protein, 12 aa, Artificial Sequence
  Description of Artificial Sequence Synthetic peptide
HPWEVMPDLYFY

SEQ ID NO: 36 - protein, 12 aa, Artificial Sequence
  Description of Artificial Sequence Synthetic peptide
VMPDLYFYRDPE

SEQ ID NO: 37 - protein, 12 aa, Artificial Sequence
  Description of Artificial Sequence Synthetic peptide
LYFYRDPEEIEK

SEQ ID NO: 38 - protein, 12 aa, Artificial Sequence
  Description of Artificial Sequence Synthetic peptide
RDPEEIEKEEQA

SEQ ID NO: 39 - protein, 12 aa, Artificial Sequence
  Description of Artificial Sequence Synthetic peptide
EIEKEEQAAAEK

SEQ ID NO: 40 - protein, 12 aa, Artificial Sequence
  Description of Artificial Sequence Synthetic peptide
EEQAAAEKAVTK

SEQ ID NO: 41 - protein, 12 aa, Artificial Sequence
  Description of Artificial Sequence Synthetic peptide
AAEKAVTKEEFQ

SEQ ID NO: 42 - protein, 12 aa, Artificial Sequence
  Description of Artificial Sequence Synthetic peptide
AVTKEEFQGEWT

SEQ ID NO: 43 - protein, 12 aa, Artificial Sequence
  Description of Artificial Sequence Synthetic peptide
EEFQGEWTAPAP

SEQ ID NO: 44 - protein, 12 aa, Artificial Sequence
  Description of Artificial Sequence Synthetic peptide
GEWTAPAPEFTA

SEQ ID NO: 45 - protein, 12 aa, Artificial Sequence
  Description of Artificial Sequence Synthetic peptide
APAPEFTATQPE

SEQ ID NO: 46 - protein, 12 aa, Artificial Sequence
  Description of Artificial Sequence Synthetic peptide
VPIQQFPTEDWS

SEQ ID NO: 47 - protein, 12 aa, Artificial Sequence
  Description of Artificial Sequence Synthetic peptide
EDWSAQPATEDW

SEQ ID NO: 48 - protein, 12 aa, Artificial Sequence
  Description of Artificial Sequence Synthetic peptide
AQPATEDWSAAP

SEQ ID NO: 49 - protein, 12 aa, Artificial Sequence
  Description of Artificial Sequence Synthetic peptide
TEDWSAAPTAQA

SEQ ID NO: 50 - protein, 12 aa, Artificial Sequence
  Description of Artificial Sequence Synthetic peptide
SAAPTAQATEWV

SEQ ID NO: 51 - protein, 12 aa, Artificial Sequence
  Description of Artificial Sequence Synthetic peptide
TAQATEWVGATT

SEQ ID NO: 52 - protein, 11 aa, Artificial Sequence
  Description of Artificial Sequence Synthetic peptide
TEWVGATTDWS

SEQ ID NO: 53 - protein, 16 aa, Artificial Sequence
  Description of Artificial Sequence Synthetic peptide
EEFQGEWTAPAPEFTA

SEQ ID NO: 54 - protein, 24 aa, Artificial Sequence
  Description of Artificial Sequence Synthetic peptide
QFPTEDWSAQPATEDWSAAPTAQA

SEQ ID NO: 55 - DNA, 429 bp, Artificial Sequence
  Description of Artificial Sequence Synthetic polynucleotide
atgggattcagcaggatctttctcttcctcctgtcaataactacaggtgtccactcccaggcttatctacagcagtctggggctgagctggtgaggtctggggcctcagtgaagatgtcctgcaaggcttctggctacacatttaccagttacaatatgcactgggtaaagcagacacctggacagggcctggaatggattggatatatttatcctggaaatggtggtactaactacaatcagaagttcaagggcaaggccacattgactgcagacacatcctccagcacagcctacatgcagatcagcagcctgacatctgaagactctgcggtctatttctgtgcaagggggggttattactacggtagtagctgggagctctactttgactactggggccaaggcaccactctcacagtctcctca

SEQ ID NO: 56 - protein, 143 aa, Artificial Sequence
  Description of Artificial Sequence Synthetic polypeptide
MGFSRIFLFLLSITTGVHSQAYLQQSGAELVRSGASVKMSCKASGYTFTSYNMHWVKQTPGQGLEWIGYIYPGNGGTNYNQKFKGKATLTADTSSSTAYMQISSLTSEDSAVYFCARGGYYYGSSWELYFDYWGQGTTLTVSS

SEQ ID NO: 57 - DNA, 393 bp, Artificial Sequence
  Description of Artificial Sequence Synthetic polynucleotide
atgaagttgcctgttaggctgttggtgctgatgttctggattcctgcttccagcagtgatgttttgatgacccaaactccactctccctgcctgtcagtcttggagatcaagcctccatctcttgcagatctagtcagagcattgtacatagtaatggaaacacctatttagaatggtacctgcagaaaccaggccagtctccaaagctcctgatctacaaagtttccaaccgattttctggggtcccagacaggttcagtggcagtggatcagggacagatttcacactcaagatcagcagagtggaggctgaggatctgggagtttattactgctttcaaggttcacatgttcctccgacgttcggtggaggcaccaagctggaaatcaaa

SEQ ID NO: 58 - protein, 131 aa, Artificial Sequence
  Description of Artificial Sequence Synthetic polypeptide
MKLPVRLLVLMFWIPASSSDVLMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSHVPPTFGGGTKLEIK

SEQ ID NO: 59 - DNA, 390 bp, Artificial Sequence
  Description of Artificial Sequence Synthetic polynucleotide
atggctgtcctggcattactcttctgcctggtaacattcccaagctgtatcctttcccaggtgcagctgaaggagtcaggacctggcctggtggcgccctcacagagcctgtccatcacatgcaccgtctcagggttctcattaaccgcctatggtgtaaactgggttcgccagcctccaggaaagggtctggagtggctgggaatgatatggggtaatggagacacagactataattcagctctcaaatccagactgagcatcagcaaggacaactccaagagccaagttttcttaaagatgaacagtctgcaaactgatgacacagccaggtactactgtgccagatatggctactggggccaagggactctggtcactgtctctgca

SEQ ID NO: 60 - protein, 130 aa, Artificial Sequence
  Description of Artificial Sequence Synthetic polypeptide
MAVLALLFCLVTFPSCILSQVQLKESGPGLVAPSQSLSITCTVSGFSLTAYGVNWVRQPPGKGLEWLGMIWGNGDTDYNSALKSRLSISKDNSKSQVFLKMNSLQTDDTARYYCARYGYWGQGTLVTVSA

SEQ ID NO: 61 - DNA, 393 bp, Artificial Sequence
  Description of Artificial Sequence Synthetic polynucleotide
atgagtcctgcccagttcctgtttctgttagtgctctggattcgggaaaccaacggtgatgttgtgatgacccagactccactcactttgtcggttacccttggacaaccagcctccatctcttgcaagtcaagtcagagcctcttagatagtgatggaaagacatatttgaattggttgttacagaggccaggccagtctccaaagcgcctaatctatctggtgtctaaagtggactctggagtccctgacaggttcactggcagtggatcagggacagatttcacactgaaaatcagcagagtggaggctgaggatttgggagtttattattgctggcaaggtacacattttccgttcacgttcggtgctgggaccaagctggagctgaaa

SEQ ID NO: 62 - protein, 131 aa, Artificial Sequence
  Description of Artificial Sequence Synthetic polypeptide
MSPAQFLFLLVLWIRETNGDVVMTQTPLTLSVTLGQPASISCKSSQSLLDSDGKTYLNWLLQRPGQSPKRLIYLVSKVDSGVPDRFTGSGSGTDFTLKISRVEAEDLGVYYCWQGTHFPFTFGAGTKLELK

SEQ ID NO: 63 - DNA, 393 bp, Artificial Sequence
  Description of Artificial Sequence Synthetic polynucleotide
atgaactttgtgctcagcttgattttccttgccctcattttaaaaggtgtccagtgtgaagtgcagctagtggagtctgggggaggcttagtgaagcctggagggtccctgaaactctcctgtgcagcctctggattcactttcagtagctataccatgtcttgggttcgccagactccggagaagaggctggagtgggtcgcaaccattagtagtggtggtacttacacctactatccagacagtgtgaagggtcgattcaccatctccagagacaatgccaagaacaccctgtacctgcaaatgagcagtctgaggtctgaggacacggccatgtattactgtgcaagactgaggtactggggccaagggactctggtcactgtctctgca

SEQ ID NO: 64 - protein, 131 aa, Artificial Sequence
  Description of Artificial Sequence Synthetic polypeptide
MNFVLSLIFLALILKGVQCEVQLVESGGGLVKPGGSLKLSCAASGFTFSSYTMSWVRQTPEKRLEWVATISSGGTYTYYPDSVKGRFTISRDNAKNTLYLQMSSLRSEDTAMYYCARLRYWGQGTLVTVSA

SEQ ID NO: 65 - DNA, 393 bp, Artificial Sequence
  Description of Artificial Sequence Synthetic polynucleotide
atgagtcctgcccagttcctgtttctgttagtgctctggattcgggaaaccaacggtgatgttgtgatgacccagactccactcactttgtcggttaccattggacaaccagcctccatctcttgcaagtcaggtcagagcctcttagatagtgatggaaagacatatttgaattggttgttacagaggccaggccagtctccaaagcgcctaatctatctggtgtctaaactggactctggagtccctgacaggttcactggcagtggatcagggacagatttcacactgaaaatcagcagagtggaggctgaggatttgggagtttattattgctggcaaggtacacattttcctcagacgttcggtggaggcaccaagctggaaatcaaa

SEQ ID NO: 66 - protein, 131 aa, Artificial Sequence
  Description of Artificial Sequence Synthetic polypeptide
MSPAQFLFLLVLWIRETNGDVVMTQTPLTLSVTIGQPASISCKSGQSLLDSDGKTYLNWLLQRPGQSPKRLIYLVSKLDSGVPDRFTGSGSGTDFTLKISRVEAEDLGVYYCWQGTHFPQTFGGGTKLEIK

SEQ ID NO: 67 - DNA, 390 bp, Artificial Sequence
  Description of Artificial Sequence Synthetic polynucleotide
atggctgtcctggcactactcctctgcctggtggctttcccaagctgtaccctgtcccaggtgcaactgaaggagtcaggacctggcctggtggcgccctcacagagcctgtccattacctgcactgtctctgggttctcattaaccagctatgatataagctggattcgccagccaccaggaaagggtctggagtggcttggagtaatatggactggtggaggcacaaattataattcagctttcatgtccagactgagcatcagcaaggacaactccaagagccaagttttcttaaaaatgaacagtctgcaaactgatgacacagccatatattactgtgtaagctttgtttactggggccaagggactctggtcactgtctctgca

SEQ ID NO: 68 - protein, 130 aa, Artificial Sequence
  Description of Artificial Sequence Synthetic polypeptide
MAVLALLLCLVAFPSCTLSQVQLKESGPGLVAPSQSLSITCTVSGFSLTSYDISWIRQPPGKGLEWLGVIWTGGGTNYNSAFMSRLSISKDNSKSQVFLKMNSLQTDDTAIYYCVSFVYWGQGTLVTVSA

SEQ ID NO: 69 - DNA, 393 bp, Artificial Sequence
  Description of Artificial Sequence Synthetic polynucleotide
atgaagttgcctgttaggctgttggtgctgatgttctggattcctgcttccagcagtgatgttgtgatgacccaaactccactctccctgcctgtcagtcttggagatcaagcctccatctcttgcagatctagtcagagccttgtacacagtaatggaaacacctatttacattggtacctgcagaagccaggccagtctccaaagctcctgatctacaaagtttccaaccgattttctggggtcccagacaggttcagtggcagtggatcagggacagatttcacactcaagatcagcagagtggaggctgaggatctgggagtttatttctgctctcaaagtacacatgttccgtggacgttcggtggaggcaccaagctggaaatcaaa

SEQ ID NO: 70 - protein, 131 aa, Artificial Sequence
  Description of Artificial Sequence Synthetic polypeptide
MKLPVRLLVLMFWIPASSSDVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQSTHVPWTFGGGTKLEIK

(19) United States
(12) Patent Application Publication
(10) Pub. No.: US 2016/0229918 A1
(43) Pub. Date: Aug. 11, 2016

(54) TUMOR SELECTIVE ANTIBODIES SPECIFIC TO ONCOFETAL ANTIGEN/IMMATURE LAMININ RECEPTOR PROTEIN

(71) Applicants: BENOVUS BIO, INC., Atlanta, GA (US); SOUTH ALABAMA MEDICAL SCIENCE FOUNDATION, Mobile, AL (US)

(72) Inventors: Kent J. Johnson, Ann Arbor, MI (US); Alton C. Morgan, Savannah, GA (US); Joseph A. Sinkule, Paradise Valley, AZ (US); James Varani, Ann Arbor, MI (US); Joseph H. Coggin (Deceased), Mobile, AL (US); James W. Rohrer (Deceased), Mobile, AL (US); Adel L. Barsoum, Mobile, AL (US)

(73) Assignees: Benovus Bio, Inc., Atlanta, GA (US); South Alabama Medical Science Foundation, Mobile, AL (US)

(21) Appl. No.: 14/904,312

(22) PCT Filed: Jul. 11, 2014
(86) PCT No.: PCT/US2014/046424

§ 371 Date: Jan. 11, 2016

Related U.S. Application Data
(60) Provisional application No. 61/845,155, filed on Jul. 11, 2013.

(51) Int. Cl. C07K 16/30; G01N 33/574

(52) U.S. Cl. CPC C07K 16/30; C07K 16/303; C07K 16/3023; C07K 16/3015; C07K 16/3046; C07K 16/3069; C07K 16/3061; G01N 33/57476; C07K 2317/565; C07K 2317/20; C07K 2317/34; C07K 2317/73; C07K 2317/92

(57) ABSTRACT

Disclosed are high affinity antibodies or antigen binding fragments thereof, which bind an epitope that lies within the C terminal region of oncofetal antigen (OFA)/immature laminin receptor protein (iLRP), and which do not substantially cross-react with mature OFA/LRP. The antibodies may be conjugated to cytotoxic moieties to enhance their therapeutic efficacy. Methods of making the antibodies and therapeutic and diagnostic uses thereof are also disclosed.

### CROSS-REFERENCE TO RELATED APPLICATION

The present application claims the benefit of the filing date of U.S. Provisional Application No. 61/845,155, filed Jul. 11, 2013, entitled HIGH-AFFINITY, TUMOR SELECTIVE ANTIBODIES SPECIFIC TO ONCOFETAL ANTIGEN/IMMATURE LAMININ RECEPTOR PROTEIN, the disclosure of which is hereby incorporated herein by reference.

### BACKGROUND OF THE INVENTION

The 32-44kD oncofetal antigen (OFA), also known as 37Kd OFA or immature laminin receptor protein (iLRP), is a monomeric non-acylated oncofetal antigen. It is present in all embryonic and early fetal cells in humans, including inbred pregnancies. Subsequently, in mid-to-late gestation, 37kD OFA/iLRP ceases to be expressed as an active antigen. Rather, it becomes dimerized, forming the 67kD mature laminin receptor protein (mLRP) which is non-immunogenic. Mature LRP is expressed at varying levels on some normal adult cells, and is believed to function so as to enable these differentiated adult cells to egress through laminin basement membranes in normal tissues and vessel walls.

There is experimental evidence that following early neoplastic cell transformation, OFA/iLRP is re-expressed on cancer cells. See generally, Coggin et al., Mod. Asp. Immunobiol. 16:27-34 (2005). This protein has thus been implicated in several aspects of cancer progression, including tumor invasiveness, metastasis, and growth. See, Zelle-Rieser, et al., J. Urol. 165(5):1705-9 (2001).

In addition to data demonstrating the expression of OFA/iLRP on malignant tumors, there is also evidence that targeting OFA/iLRP directly can influence tumor growth and/or spread. Specifically, it has been shown that dendritic cells primed with OFA/iLRP or transfected with RNA specific to OFA/iLRP can induce a T-cell immune response with the capacity to suppress growth of OFA/iLRP-positive hematological malignancies in syngeneic mouse models. See, Rohrer et al., J. Immunol. 176:2844-56 (2006); and Siegel et al., Blood 102:4416-23 (2003). See also U.S. Pat. Nos. 6,534,060 and 7,718,762, and U.S. Patent Application Publication 2013/0052211 A1.

There is also indirect evidence for a humoral response. The presence of antibodies to OFA/iLRP has been reported in the serum of some leukemia patients. See, e.g., Siegel et al., Leukemia 22:2115-18 (2008); and Friedrichs et al., Leukemia Res. 35(6): 721-29 (2010). Serum from patients with antibodies to OFA/iLRP lysed OFA/iLRP-positive tumor cells in vitro, while serum from subjects without detectable antibody did not. Most importantly, individuals who mounted a humoral response had a more favorable prognosis than those who did not. See id. While these findings are consistent with a role antibody-dependent anti-tumor activity, there is no direct evidence that targeting OFA/iLRP with exogenous antibody can be used therapeutically. An early report of monoclonal antibodies specific to embryonic antigens such as OFA, is described in U.S. Pat. No. 4,686,180. There have been several subsequent reports of the production of monoclonal antibodies against OFA/iLRP. Aarli et al., Am. J. Rep. Immunol. 38(5):313-319 (1997); Lee et al., J. Cell Biol. 117(3):671-78 1992); Liotta et al., Exp. Cancer Res. 156(1):117-26 (1985); Sanz et al., Cancer Therapy Immunother: 52:643-47 (2003); and Rahman et al., J. Natl. Cancer Int. 81(23):1794-1800 (1989). There are reports that anti-OFA antibodies react with both the OFA/iLRP and the mature LRP. Sobel et al., Semin. Cancer Biology 4(5):311-317 (1993); Cloce et al., J. Natl. Cancer Int. 2(83):29-36 (1991); and Coggin et al., Anticancer Res. 25(3):2345-55 (2005). Thus, known antibodies may lack the requisite specificity for purposes of therapeutic use. More recently, U.S. Patent Application Publication 2010/0247536 A1, to O11e, describes monoclonal antibodies specific to OFA/iLRP and which purportedly do not bind mature LRP. However, these antibodies may not have adequate affinity for the target for clinical purposes.

Accordingly, a need exists to develop antibodies that bind with relatively high affinity to OFA/iLRP, and do not bind mLRP, and which can be used alone or in conjunction with other therapeutic modalities, to develop effective therapies for cancer.

### SUMMARY OF THE INVENTION

Applicants have discovered antibodies that bind to one or more epitopes within the immunogenic C-terminal region of OFA/iLRP with relatively high affinity, and which inhibit proliferation of cancer cells. The antibodies do not substantially cross-react with mature OFA/LRP or with non-cancerous cells. Their specificity, coupled with a relatively high affinity, make them particularly attractive for use in the diagnosis and treatment of cancer.

Accordingly, one aspect of the present invention provides antibodies or antigen-binding fragments thereof, which bind an epitope that lies within the C-terminal region of OFA/iLRP and which do not substantially cross-react with mature OFA/LRP. Variable regions of the antibodies of the present invention contain the three heavy chain and the three light chain complementarity determining regions (CDRs) as illustrated in any of FIGS. 1-4 (or variants thereof), and as described herein below.

As demonstrated in working examples, antibodies of the present invention possess the ability to block OFA/iLRP-induced signaling pathways and/or become internalized in a cancer cell. Antibody-dependent inhibition of cell growth and proliferation is a property rare among antibodies specific to tumor-associated antigens, including known antibodies specific to OFA. Antibody-dependent antigen internalization is less rare but not all antibodies induce internalization and even for those that do, the rate of internalization varies. In certain embodiments, this property may be exploited by conjugating the antibody to a toxin or cytotoxic moiety. When used in cancer therapy, the conjugated antibody may exhibit enhanced toxicity toward cancer cells. As also demonstrated in the working examples, antibodies of the present invention significantly reduced blood tumor burden in an animal model of hematological cancer.

Another aspect of the present invention provides nucleic acids encoding the antibodies or fragments thereof, recombinant vectors and host cells comprising the nucleic acids, and methods of producing the antibodies using the host cells.

Further aspects of the present invention provide pharmaceutical compositions and kits comprising the antibodies.

Yet further aspects of the present invention are directed to methods of treating cancer, diagnosing cancer, and monitoring cancer therapy, in subjects (including humans and animals) using the antibodies of the invention.

### BRIEF DESCRIPTION OF THE DRAWINGS

FIG. 1 shows the amino acid sequences and corresponding nucleic acid sequences of the heavy and light chains of the variable region of an antibody of the present invention (referred to herein as “BV-06”), wherein the complementarity determining regions (CDRs) are underscored.

FIG. 2 shows the amino acid sequences and corresponding nucleic acid sequences of the heavy and light chains of the variable region of an antibody of the present invention (referred to herein as “BV-12”), wherein the CDRs are underscored.

FIG. 3 shows the amino acid sequences and corresponding nucleic acid sequences of the heavy and light chains of the variable region of an antibody of the present invention (referred to herein as “BV-15”), wherein the CDRs are underscored.

FIG. 4 shows the amino acid sequences and corresponding nucleic acid sequences of the heavy and light chains of the variable region of an antibody of the present invention (referred to herein as “BV-27”), wherein the CDRs are underscored.

FIG. 5 is a graph that shows a typical set of ELISA binding curves using various concentrations of MAb BV-27 against increasing amounts of recombinant OFA (rOFA).

FIGS. 6A-B are graphs that show binding of monoclonal antibodies of the present invention to HL-60 and K562 cells (as compared to controls).

FIGS. 7A-C are graphs that show (A) flow cytometry patterns for polystyrene beads with precisely known amounts of CD5 antigen, wherein the insert (calibration curve) shows the relationship between theoretical fluorescence and the fluorescence actually attained, and the middle (B) and lower (C) panels demonstrate fluorescence using the control IgG2a antibody and BV-27.

FIGS. 8A-D are histograms that show HL60 binding of MAb BV-12, wherein the upper panels (A and B) show histograms from low concentrations (0.01 μg) of a class-matched IgG and the specific MAb BV-12, and wherein the histogram generated with the control IgG was used to determine background binding, and wherein the lower panels (C and D) show histograms from high antibody concentrations (5 mg).

FIGS. 9A-E are Western blots of four inventive monoclonal antibodies and a non-inventive monoclonal antibody (BV-19) generated to rOFA (recombinant OFA), with respect to Lane a: K562 cells; Lane b: NB4 cells; Lane c: HC38 breast carcinoma (triple negative) cells; Lane d: normal fibroblast cells; Lane e: normal keratinocytes; and Lane f: Immortalized keratinocytes.

FIGS. 10A-E are photographs of confocal fluorescent microscopy with a triple-negative breast cancer cell line (HCC38), normal human epithelial cells and fibroblasts, using four inventive monoclonal antibodies and a non-inventive monoclonal antibody (BV-19); FIGS. 10F and G show differential immunohistological staining using BV-15 of ductal cell carcinoma of the breast compared with normal breast tissue, respectively.

FIGS. 11A-B are graphs that show percent attachment of MCA-1315 cells to laminin-coated (A) and fibronectin-coated (B) culture dishes, as a function of time.

FIGS. 12A-B are bar graphs that show inhibition of MCA-1315 cell attachment to laminin-coated (A) and fibronectin-coated (B) culture dishes in the presence of inventive antibodies, non-inventive antibodies and control, as a function of time.

FIGS. 13A-D are photographs showing microscopic appearance of MCA-1315 cells on laminin-coated dishes, in the presence of an inventive monoclonal antibody, a non-inventive antibody (2C6) and controls. FIG. 13E shows the amino acid sequences and corresponding nucleic acid sequences of the heavy and light chains of the variable region of 2C6, wherein the leader sequences are in bold and the CDRs are underscored.

FIG. 14 is a bar graph that shows inhibition of proliferation of MCA-1315 cells in the presence of BV-27.

FIGS. 15A-J are graphs that show cross-competition among inventive monoclonal antibodies and a non-inventive monoclonal antibody (BV-19) using ELISA.

FIGS. 16A-E are bar graphs that show reactivity of inventive monoclonal antibodies and a non-inventive monoclonal antibody to OFA synthetic peptides spanning the C-terminal region of OFA.

FIGS. 17A-D are bar graphs that show cross-competition among inventive monoclonal antibodies measured via an intact cell assay.

FIGS. 18A-B are bar graphs that show effects of increasing concentration of biotinylated BV-27 on enhancement of binding of BV-15 to HL-60 cells.

FIGS. 19A-B are graphs that show effect of BV-27 and BV-15, respectively, on primary tumor (A20) growth in syngeneic mice, compared to a control.

FIGS. 20A-C are bar graphs that show suppression of liver tumor formation in syngeneic mice by BV-27 compared to a control.

FIGS. 21A-B are bar graphs that show suppression of blood-borne tumor colony formation in syngeneic mice by BV-27 compared to a control.

FIGS. 22A-C are bar graphs that show suppression of lung tumor formation in syngeneic mice by BV-15 and BV-27 compared to a control.

FIG. 23 is a graph showing a standard curve plotting the log concentration of rOFA standards (x-axis) against optical density (y-axis).

FIG. 24 is a bar graph showing levels of OFA in serum of dogs with acute lympho-sarcoma as compared to animals in remission, healthy controls and control animals having various inflammatory conditions.

### DETAILED DESCRIPTION

The present invention provides antibodies or fragments thereof specific for their corresponding epitopes that reside within the C-terminus, which is the laminin binding region of OFA/iLRP, and which have CDR sequences as described herein. As demonstrated in the working examples, certain of the inventive antibodies also block OFA/iLRP from binding to laminin (e.g., BV-15), and inhibit OFA/iLRP-induced signaling pathways (e.g., BV-27), inhibit tumor proliferation, and inhibit tumor growth in vitro and in vivo (e.g., BV-15 and BV-27).

As used herein, “OFA” and “iLRP” are used interchangeably along with “OFA/iLRP”, and refer to the full-length consensus 295-amino acid protein, with variability in positions 18, 155, 241, and 293 as shown below (wherein “Mu” refers to murine and “Hu” refers to human), the sequence of which is described in U.S. Pat. No. 7,718,762, to Coggin et al.

| | | |
|---|---|---|
| Mu iLRP | M S G A L D V L Q M K E E D V L K L L A | 20 |
| Hu iLRP | - - - - - - - - - - - - - - - - - F - - | |
| Mu OFA | - - - - - - - - - - - - - - - - - F - - | |
| | A G T H L G G T N L D F Q M E Q Y I Y K | 40 |
| Hu iLRP | - - - - - - - - - - - - - - - - - - - - | |
| Mu OFA | - - - - - - - - - - - - - - - - - - - - | |
| | R K S D G I Y I I N L K R T W E K L L L | 60 |
| Hu iLRP | - - - - - - - - - - - - - - - - - - - - | |
| Mu OFA | - - - - - - - - - - - - - - - - - - - - | |
| | A A R A I V A I E N P A D V S V I S S R | 80 |
| Hu iLRP | - - - - - - - - - - - - - - - - - - - - | |
| Mu OFA | - - - - - - - - - - - - - - - - - - - - | |
| Mu iLRP | N T G Q R A V L K F A A A T G A T P I A | 100 |
| Hu iLRP | - - - - - - - - - - - - - - - - - - - - | |
| Mu OFA | - - - - - - - - - - - - - - - - - - - - | |
| Mu iLRP | G R F T P G T F T N Q I Q A A F R E P R | 120 |
| Hu iLRP | - - - - - - - - - - - - - - - - - - - - | |
| Mu OFA | - - - - - - - - - - - - - - - - - - - - | |
| Mu iLRP | L L V V T D P R A D H Q P L T E A S Y V | 140 |
| Hu iLRP | - - - - - - - - - - - - - - - - - - - - | |
| Mu OFA | - - - - - - - - - - - - - - - - - - - - | |
| Mu iLRP | N L P T I A L C N T D S P L A Y V D I A | 160 |
| Hu iLRP | - - - - - - - - - - - - - - R - - - - - | |
| Mu OFA | - - - - - - - - - - - - - - R - - - - - | |
| Mu iLRP | I P C N N K G A H S V G L M W W M L A R | 180 |
| Hu iLRP | - - - - - - - - - - - - - - - - - - - - | |
| Mu OFA | - - - - - - - - - - - - - - - - - - - - | |
| Mu iLRP | E V L R M R G T I S R E H P W E V M P D | 200 |
| Hu iLRP | - - - - - - - - - - - - - - - - - - - - | |
| Mu OFA | - - - - - - - - - - - - - - - - - - - - | |
| Mu iLRP | L Y F Y R D P E E I E K E E Q A A A E K | 220 |
| Hu iLRP | - - - - - - - - - - - - - - - - - - - - | |
| Mu OFA | - - - - - - - - - - - - - - - - - - - - | |
| Mu iLRP | A V T K E E F Q G E W T A P A P E F T A | 240 |
| Hu iLRP | - - - - - - - - - - - - - - - - - - - - | |
| Mu OFA | - - - - - - - - - - - - - - - - - - - - | |
| Mu iLRP | A Q P E V A D W S E G V Q V P S V P I Q | 260 |
| Hu iLRP | T - - - - - - - - - - - - - - - - - - - | |
| Mu OFA | A - - - - - - - - - - - - - - - - - - - | |
| Mu iLRP | Q F P T E D W S A Q P A T E D W S A A P | 280 |
| Hu iLRP | - - - - - - - - - - - - - - - - - - - - | |
| Mu OFA | - - - - - - - - - - - - - - - - - - - - | |
| Mu iLRP | T A Q A T E W V G A T T E W S | 295 |
| Hu iLRP | - - - - - - - - - - - - D - - | |
| Mu OFA | - - - - - - - - - - - - E - - | |

### Amino Acid Abbreviations:

Alanine A
Arginine R
Asparagine M
Aspartic Acid D
Cysteine C
Glutamine Q
Glutamic Acid E
Glycine G
Histidine H
Isoleucine I
Leucine L
Lysine K
Methionine M
Phenylalanine F
Proline P
Serine S
Threonine T
Tryptophan W
Tyrosine Y
Valine V

As used herein, the term “antibody” includes intact immunoglobulins and antigen-binding portions or fragments thereof that retain the binding specificity and affinity for the antigen. An IgG “immunoglobulin” is a tetrameric molecule. In a naturally-occurring IgG immunoglobulin, each tetramer is composed of two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. For IgG, the CH2 and CH3 domains of each heavy chain define a constant region primarily responsible for effector function. The light chains of antibodies from any vertebrate species can be assigned to one of two clearly distinct types, called kappa (κ) and lambda (λ), based on the amino acid sequences of their constant domains. The variable regions of kappa and lambda light chains are referred to herein as Vκ and vλ, respectively. The expression VL, as used herein, is intended to include both the variable regions from kappa-type light chains (Vκ) and from lambda-type light chains (Vλ). Heavy chains are classified as μ, δ, γ, α or ε, and define the antibody's isotype as IgM, IgD, IgG, IgA, and IgE, respectively. Several of these may be further divided into subclasses, e.g., IgG1, IgG2, IgG3, IgG4, IgA1 and IgA2. Within light and heavy chains, the variable and constant regions are joined by a “J” region of about 12 or more amino acids, with the heavy chain also including a “D” region of about 10 more amino acids. See generally, Fundamental Immunology Ch. 7 (Paul, W., ed., 2nd ed. Raven Press, N.Y. (1989)). The variable regions of each light/heavy chain pair form the antibody binding site such that an intact immunoglobulin has two binding sites. Immunoglobulin chains exhibit the same general structure of relatively conserved framework regions (FR) joined by three hypervariable regions, also called complementarity determining regions or CDRs. The CDRs from the two chains of each pair are aligned by the framework regions, enabling binding to a specific epitope. From N-terminus to C-terminus, both light and heavy chains comprise the domains FR1, CDR1, FR2, CDR2, FR3, CDR3, and FR4. The assignment of amino acids to each domain is in accordance with the definitions of Kabat Sequences of Proteins of Immunological Interest (National Institutes of Health, Bethesda, Md. (1987 and 1991), or Chothia & Lesk, J. Mol. Biol. 196:901-917 (1987); Chothia et al., Nature 342:878-883 (1989)). The present invention includes antibodies of any of the aforementioned classes or subclasses, including any of the different constant domains.

The antibodies of the present invention include “monoclonal antibodies,” which as used herein, refers to an antibody obtained from a population of substantially homogeneous antibodies, e.g., the individual antibodies comprising the population are substantially identical except for possible naturally occurring mutations or minor post-translational variations that may be present. Monoclonal antibodies are highly specific, being directed against a single antigenic site (also known as determinant or epitope). This is in contrast to polyclonal antibodies that typically include different antibodies directed against different antigenic determinants. The modifier “monoclonal” indicates the character of the antibody as being obtained from a substantially homogeneous population of antibodies, and is not to be construed as requiring production of the antibody by any particular method. As disclosed herein, the antibodies of the present invention include murine, chimeric, human and humanized antibodies.

Antibody specificity refers to selective recognition of the antibody for a particular epitope of an antigen. The terms “epitope” and “antigenic determinant” are used interchangeably herein and refer to that portion of an antigen capable of being recognized and specifically bound by a particular antibody. When the antigen is a polypeptide, epitopes can be formed both from contiguous amino acids and noncontiguous amino acids juxtaposed by tertiary folding of a protein. Epitopes formed from contiguous amino acids are typically retained upon protein denaturing, whereas epitopes formed by tertiary folding are typically lost upon protein denaturing. An epitope typically includes at least 3, and more usually, at least 5 or 8-16 amino acids in a unique spatial conformation.

The antibodies of the present invention are selective or specific for their respective epitope within the C-terminal region of OFA/iLRP. As used herein, the term “C-terminal region of OFA” refers to amino acids 177-295 of OFA. These epitopes are presented on the surface of cancer cells. The antibodies may exhibit both species and molecule selectivity, or may be selective with respect to the molecule only and thus bind OFA/iLRP of more than one species. The antibodies of the invention may bind mouse, rat, rabbit, cat, dog, pig, horse, cow, monkey, and/or human OFA/iLRP. In one embodiment, the antibody binds to human OFA/iLRP. Whether an antibody specifically binds OFA/iLRP can be determined, e.g., by a binding assay such as an ELISA, employing a panel of antigens. As demonstrated in the working examples, the inventive antibodies are selective or specific for at least one epitope in the C-terminal region of OFA/iLRP, e.g., amino acid residues 217-232 (AAEKAVTKEEFQGEWT), residues 261-272 (QFPTEDWSAQPA) and residues 261-276 (QFPTEDWSAQPATEDW).

That an antibody “selectively binds” or “specifically binds” to an epitope or antigen means that the antibody reacts or associates more frequently, more rapidly, with greater duration, with greater affinity, or with some combination of the above to an epitope than with alternative substances, including unrelated proteins. The antibodies of the present invention exhibit substantially no cross-reactivity with mature LRP, which in the context of the present invention means that binding of the antibodies with mLRP is minimal or not even detectable within the limits of the assays employed (e.g., Western blotting as demonstrated in the working examples).

Antibodies of the present invention can be monospecific or multi-specific. Monospecific antibodies bind only one antigen at one site, i.e., an epitope within the C-terminal region of OFA/iLRP. Multi-specific antibodies have two or more different antigen-binding specificities or sites. Where an antibody has more than one such specificity, the recognized epitopes can be associated with a single antigen or with more than one antigen. For example, hybrid antibodies are immunoglobulin molecules in which pairs of heavy and light chains from antibodies with different antigenic determinant regions are assembled together so that two different epitopes (or two different antigens) can be recognized and bound. Thus, in some embodiments, a multi-specific (e.g., bispecific) antibody has one combining site from an inventive anti-OFA antibody and a second site directed to a second antigen to improve targeting to T-cells etc.

As used herein, an “isolated” or “purified” antibody includes an antibody that (1) has been partially, substantially, or fully purified from a mixture of components; (2) is monoclonal; (3) is free of other proteins from the same species; (4) is expressed by a cell from a different species; or (5) does not occur in nature. Contaminant components of its natural environment are materials which would interfere with diagnostic or therapeutic uses for the antibody, and may include enzymes, hormones, and other proteinaceous or non-proteinaceous solutes. Examples of isolated antibodies include an antibody that has been affinity purified, an antibody that has been made by a hybridoma or other cell line in vitro, and a human antibody derived from a transgenic mouse or bacteriophage.

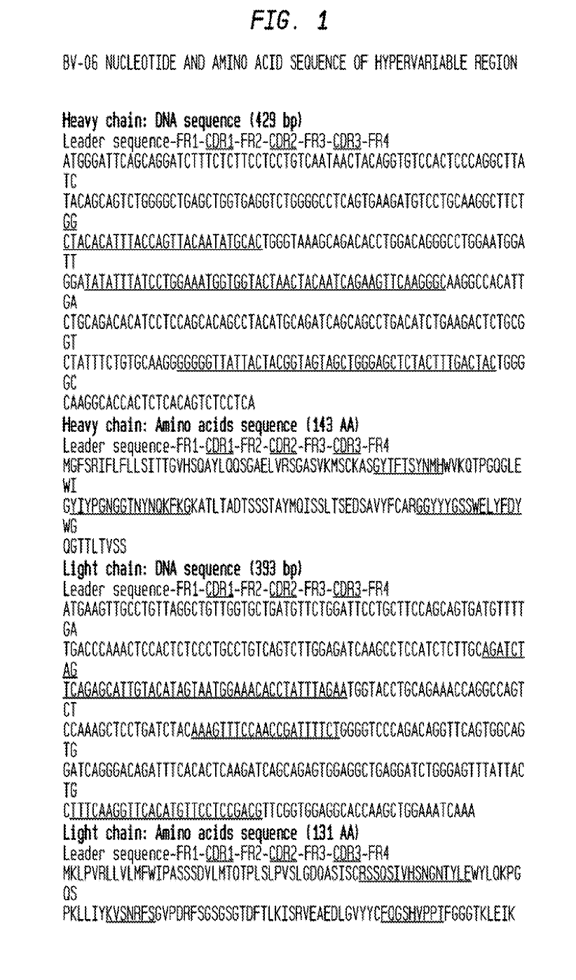

In some embodiments of the present invention, the antibodies (e.g., murine, chimeric, humanized and human) include three CDRs in the heavy chain which have the sequences GYTFTSYNMH, YIYPGNGGTNYNQKFKG, and GGYYYGSSWELYFDY, and three CDRs in the light chain which have the sequences RSSQSIVHSNGNTYLE, KVSNRFS, and FQGSHVPPT. Exemplary amino acid sequences of a heavy variable chain and a light variable chain (along with the corresponding nucleic acid sequences) that contain these CDRs are set forth in FIG. 1. A murine antibody containing the variable region illustrated in FIG. 1 and which contains an IgG2a murine constant region, is referred to herein as monoclonal antibody “BV-06.”

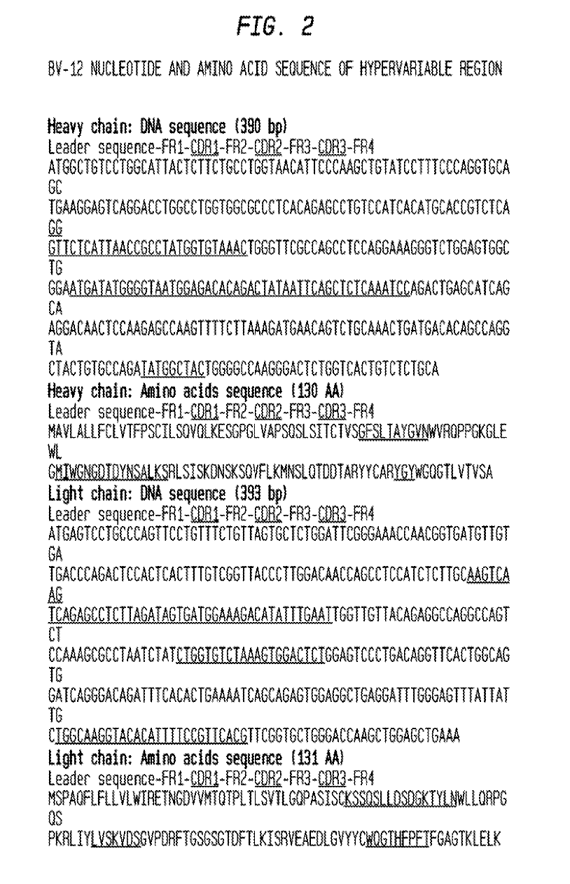

In some embodiments of the present invention, the antibodies (e.g., murine, chimeric, humanized and human) include three CDRs in the heavy chain which have the sequences GFSLTAYGVN, MIWGNGDTDYNSALKS, and YGY, and three CDRs in the light chain which have the sequences KSSQSLLDSDGKTYLN, LVSKVDS, and WQGTHFPFT. Exemplary amino acid sequences of a heavy variable chain and a light variable chain (along with the corresponding nucleic acid sequences) that contain these CDRs are set forth in FIG. 2. A murine antibody containing the variable region illustrated in FIG. 2 and which contains an IgG2a murine constant region, is referred to herein as monoclonal antibody “BV-12.”

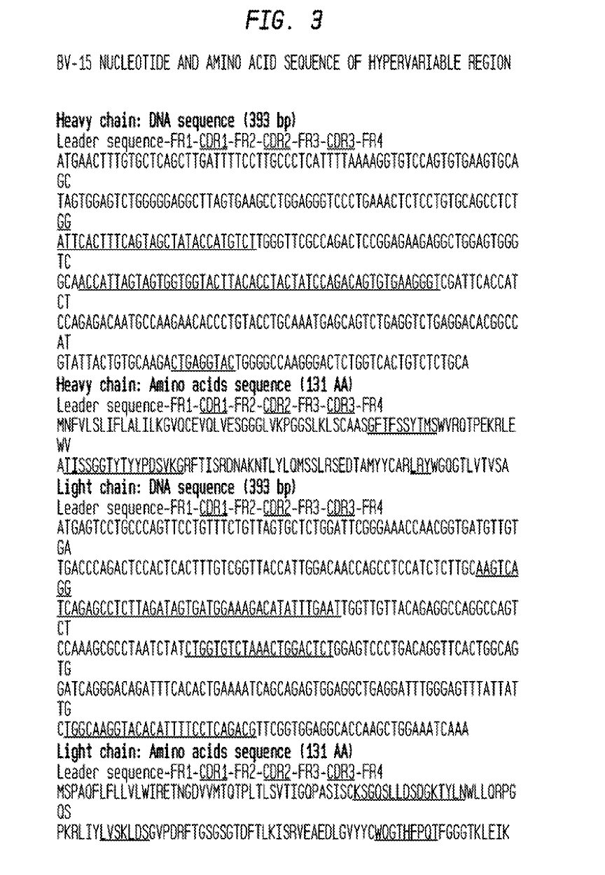

In some embodiments of the present invention, the antibodies (e.g., murine, chimeric, humanized and human) include three CDRs in the heavy chain which have the sequences GFTFSSYTMS, TISSGGTYTYYPDSVKG, and LRY, and three CDRs in the light chain which have the sequences KSGQSLLDSDGKTYLN, LVSKLDS, and WQGTHFPQT. Exemplary amino acid sequences of a heavy variable chain and a light variable chain (along with the corresponding nucleic acid sequences) that contain these CDRs are set forth in FIG. 3. A murine antibody containing the variable region illustrated in FIG. 3 and which contains an IgG2a murine constant region, is referred to herein as monoclonal antibody “BV-15.”

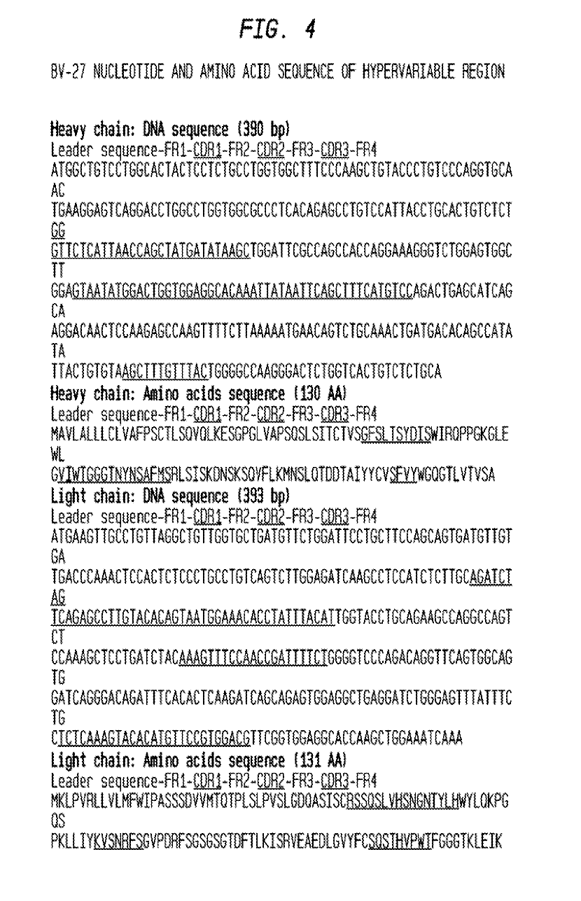

In some embodiments of the present invention, the antibodies (e.g., murine, chimeric, humanized and human) include three CDRs in the heavy chain which have the sequences GFSLTSYDIS, VIWTGGGTNYNSAFMS, and SFVY, and three CDRs in the light chain which have the sequences RSSQSLVHSNGNTYLH, KVSNRFS, and SQSTHVPWT. Exemplary amino acid sequences of a heavy variable chain and a light variable chain (along with the corresponding nucleic acid sequences) that contain these CDRs are set forth in FIG. 4. A murine antibody containing the variable region illustrated in FIG. 4 and which contains an IgG2a murine constant region, is referred to herein as monoclonal antibody “BV-27.”

The term “antibodies,” as used herein, also includes “chimeric” antibodies in which the amino acid sequence of the immunoglobulin molecule is derived from two or more species, e.g., the variable region, is identical with or homologous to corresponding sequences in antibodies derived from a mouse or rat, while the remainder of the chain(s), e.g., the constant region, is identical with or homologous to corresponding sequences in antibodies derived from another species (e.g., human). Alternatively, “chimeric” antibodies may refer to antibodies derived from 2 or more antibody classes or subclasses, e.g., CH1 and hinge of human IgG1, CH2 of IgG3, most of CH3 of IgG3 and terminal portion of CH3 of IgG1 (Natsume A et al. 2008), so long as they bind the antigen. Thus, the present invention includes, for example, chimeric antibodies comprising a chimeric heavy chain and/or a chimeric light chain. For example, the chimeric heavy chain may comprise any of the heavy chain variable (VH) regions described herein or mutants or variants thereof, fused to a heavy chain constant region of a human antibody. The chimeric light chain may comprise any of the light chain variable (VL) regions described herein or mutants or variants thereof, fused to a light chain constant region of a human antibody. Thus, in some embodiments, the chimeric antibodies contain the light and heavy chain variable domains of BV-6, BV-12, BV-15, or BV-27.

Antibodies of the invention also include “humanized antibodies”, which refer to forms of non-human (e.g., murine) antibodies that are specific immunoglobulin chains, chimeric immunoglobulins or fragments thereof that contain minimal non-human sequences. Typically, humanized antibodies are human antibody molecules having one or more complementarity determining regions (CDRs) from a non-human species and framework regions from a human immunoglobulin molecule. Often, one or more framework residues in the human framework regions will be substituted with the corresponding residue from the CDR donor antibody to restore, preferably improve, antigen binding of the humanized antibody. These framework substitutions are identified using standard techniques such as by modeling of the interactions of the CDR and framework residues to identify framework residues that may contribute to antigen binding and sequence comparison to identify unusual framework residues at particular positions. Antibodies can be humanized using a variety of techniques including CDR-grafting (Jones PT et al. 1986, Verhoeyen M et al. 1988, Riechmann L et al. 1988, SDR-grafting (specificity determining region; Gonzales NR et al. 2003, Kashmiri SV et al. 2004), veneering or resurfacing (Padlan EA 1991, Pedersen JT et al. 1994, Roguska MA et al. 1994), and framework shuffling (Wu H et al. 1999, Dall'Acqua WF et al. 2005). A variety of human framework sequences designs have been demonstrated, including a consensus human heavy and light chain framework based on the most abundant subclass families of VL κ subgroup I and VH subgroup III (Carter P et al. 1992, Presta LG et al 1993, Presta LG et al. 1997, Adams CW et al. 2006), a single human heavy and light chain framework based on sequence identity and/or molecular modeling (Queen C et al. 1989), and genetic selection from a library of homologous human heavy and light chain shuffled frameworks (Wu H et al. 1999, Dall'Acqua WF et al. 2005). In addition, human framework sequence design can be derived from expressed human antibodies (Poul MA et al. 1995, Johnson G et al. 2001) or from human germline genes (Hwang WY et al. 2005, Pelat T et al. 2008, Robert R et al. 2010).

Humanized antibodies of the present invention include the heavy and light chain CDRs contained in the antibodies described herein, e.g., BV-6, BV-12, BV-15, and BV-27.

Antibodies of the invention also include “human antibodies,” which are antibodies having variable and constant regions substantially corresponding to human germline immunoglobulin sequences (e.g., an antibody produced by a human or an antibody having an amino acid sequence corresponding to an antibody produced by a human made via known techniques). The human antibodies of the invention may include some amino acid residues not encoded by human germline immunoglobulin sequences (e.g., mutations introduced by random or site-specific mutagenesis in vitro or by somatic mutation in vivo). The human antibody can have at least one position replaced with an amino acid residue, e.g., an activity enhancing amino acid residue which is not encoded by the human germline immunoglobulin sequence. However, the term “human antibody,” as used herein, is not intended to include antibodies in which CDR sequences derived from the germline of another mammalian species, such as a mouse, have been grafted onto human framework sequences.

Human antibodies of the present invention include the heavy and light chain CDRs contained in the antibodies described herein, e.g., BV-6, BV-12, BV-15 and BV-27.

The phrase “recombinant human antibody” includes human antibodies that are prepared, expressed, created or isolated by recombinant means, such as antibodies expressed using a recombinant expression vector transfected into a host cell, antibodies isolated from a recombinant, combinatorial human antibody library, antibodies isolated from an animal that is transgenic for human immunoglobulin genes, or antibodies prepared, expressed, created or isolated by any other means that involves splicing of human immunoglobulin gene sequences to other DNA sequences. Such recombinant human antibodies have variable and constant regions derived from human germline immunoglobulin sequences.

Antigen-binding portions or fragments of the antibodies, that retain the binding specificity and affinity thereof, may be produced by recombinant DNA techniques or by enzymatic or chemical cleavage of intact antibodies. Antigen-binding portions include, inter alia, Fab, Fab', F(ab')₂, Fv, dAb, and complementarity determining region (CDR) fragments, single-chain antibodies (scFv), chimeric antibodies, diabodies, multi-specific antibodies and polypeptides that contain at least a portion of an immunoglobulin that is sufficient to confer specific antigen binding to the polypeptide. An Fab fragment is a monovalent fragment consisting of the VL, VH, CL and CH I domains; a F(ab')₂ fragment is a bivalent fragment comprising two Fab fragments linked by a disulfide bridge at the hinge region; a Fd fragment consists of the VH and CH1 domains; and a dAb fragment (Ward Dep.13, Nov. 26, 2013 et al., Nature 341:544-546 (1989)) consists of a VH domain of an antibody; An Fv fragment refers to the minimal antibody fragment that contains a complete antigen-recognition and binding site, either as two chains, in which one heavy and one light chain variable domain form a non-covalent dimer, or as a single-chain scFv in which a VL and VH regions are paired to form a monovalent molecules via a synthetic linker that enables them to be made as a single protein chain (Bird et al., Science 242:423-426 (1988) and Huston et al., Proc. Natl. Acad. Sci. USA 85:5879-5883 (1988)). Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see, e.g., Holliger et al., Proc. Natl. Acad. Sci. USA 90:6444-6448 (1993) and Poljak et al., Structure 2:1121-1123 (1994)).

In some embodiments, the fragments are scFv's wherein the VH and VL chains (e.g., of BV-06, 12, 15, or 27) are joined together such as by a short peptide linker or a disulfide bond.

Specificity of the antibodies is further conferred based on affinity and/or avidity. Avidity is related to both the affinity between an epitope with its antigen binding site on the antibody, and the valence of the antibody, which refers to the number of antigen binding sites of a particular epitope. Affinity, represented by the equilibrium constant for the dissociation of an antigen with an antibody (K_{d}), measures the binding strength between an antigenic determinant and an antibody-binding site. The lower the K_{d} value, the stronger the binding strength between an antigenic determinant and the antibody binding site. Practically, if an antibody has low inherent affinity, the specificity cannot be accurately assessed.

Antibodies of the invention bind an epitope in the C-terminal region of OFA/iLRP with a dissociation constant (K_{d}), e.g., as measured by ELISA as described herein, that generally ranges from about 1 to about 500 nm, e.g., 1, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500 (and which is inclusive of all subranges therein, e.g., from about 1-100 nm).

Antibodies of the present invention may have their affinity further increased by direct mutation (of CDR and/or framework residues), affinity maturation, phage display, or chain shuffling, in accordance with known techniques. See, e.g., Affinity maturation of monoclonal antibodies by multi-site-directed mutagenesis. Kim H Y, Stojadinovic A, Izadjoo M J. Methods Mol Biol. 2014; 1131:407-20; Yeast surface display for antibody isolation: library construction, library screening, and affinity maturation. Van Deventer JA, Wittrup KD. Methods Mol Biol. 2014; 1131:151-81; In vitro affinity maturation of a natural human antibody overcomes a barrier to in vivo affinity maturation. Li B, Fouts AE, Stengel K, Luan P, Dillon M, Liang WC, Feierbach B, Kelley RF, Hötzel I. MAbs. 2014 March-April; 6(2):437-45; The influence of antibody fragment format on phage display based affinity maturation of IgG. Steinwand M, Droste P, Frenzel A, Hust M, Dübel S, Schirrmann T. MAbs. 2014 January-February; 6(1):204-18; Mammalian cell display for the discovery and optimization of antibody therapeutics. Bowers P M, Horlick R A, Kehry M R, Neben T Y, Tomlinson G L, Altobell L, Zhang X, Macomber J L, Krapf I P, Wu B F, McConnell A D, Chau B, Berkebile A D, Hare E, Verdino P, King D J. Methods. 2014 Jan. 1; 65(1):44-56; MAPs: a database of modular antibody parts for predicting tertiary structures and designing affinity matured antibodies. Pantazes R J, Maranas C D. BMC Bioinformatics. 2013 May 30; 14(1):168; Affinity maturation by semi-rational approaches. Barderas R, Desmet J, Alard P, Casal J I. Methods Mol Biol. 2012; 907:463-86; Affinity maturation of antibodies: optimized methods to generate high-quality ScFv libraries and isolate IgG candidates by high-throughput screening. Renaut L, Monnet C, Dubreuil O, Zaki O, Crozet F, Bouayadi K, Kharrat H, Mondon P. Methods Mol Biol. 2012; 907:451-61; Femtomolar Fab binding affinities to a protein target by alternative CDR residue co-optimization strategies without phage or cell surface display. Votsmeier C, Plittersdorf H, Hesse O, Scheidig A, Strerath M, Gritzan U, Pellengahr K, Scholz P, Eicker A, Myszka D, Coco W M, Haupts U. MAbs. 2012 May-June; 4(3):341-8; Rapid selection of high-affinity binders using ribosome display. Dreier B, Plückthun A. Methods Mol Biol. 2012; 805:261-86; Synthetic single-framework antibody library integrated with rapid affinity maturation by V L shuffling. Brockmann E C, Akter S, Savukoski T, Huovinen T, Lehmusvuori A, Leivo J, Saavalainen O, Azhayev A, Lövgren T, Hellman J, Lamminmäki U. Protein Eng Des Sel. 2011 September; 24(9):691-700; In vitro affinity maturation of HuCAL antibodies: complementarity determining region exchange and RapMAT technology. Prassler J, Steidl S, Urlinger S. Immunotherapy. 2009 July; 1(4):571-83; Affinity maturation of a TNFalpha-binding affibody molecule by Darwinian survival selection. Löfdahl P A, Nygren P A. Biotechnol Appl Biochem. 2010 Mar. 5; 55(3):111-20; In vitro antibody affinity maturation targeting germline hotspots. Ho M, Pastan I. Methods Mol Biol. 2009; 525:293-308, xiv; Affinity maturation by phage display. Thie H, Voedisch B, Dübel S, Hust M, Schirrmann T. Methods Mol Biol. 2009; 525:309-22, xv; Improving antibody binding affinity and specificity for therapeutic development. Bostrom J, Lee C V, Haber L, Fuh G. Methods Mol Biol. 2009; 525:353-76, xiii; and Affinity maturation of antibodies assisted by in silico modeling. Barderas R, Desmet J, Timmerman P, Meloen R, Casal J I. Proc Natl Acad Sci USA. 2008 Jul. 1; 105(26):9029-34.

Substantially the same amino acid sequence is defined herein as a sequence with at least 70%, 75%, 80%, 85%, 86%, 87%, 88%, 89%, 90%, 91%, 92%, 93%, 94%, 95%, 96%, 97%, 98%, or 99% identity to a compared amino acid sequence, as determined by the FASTA search method in accordance with Pearson and Lipman, Proc. Natl. Acad. Sci. USA 85:2444-2448 (1988). In certain embodiments, the antibody may include a sequence at least 70%, 75%, 80%, 85%, 86%, 87%, 88%, 89%, 90%, 91%, 92%, 93%, 94%, 95%, 96%, 97%, 98%, or 99% identical to a heavy variable and/or light variable chain of the sequences illustrated in FIGS. 1-4. The amino acid differences (which may be in the constant or variable region, including the CDRs or framework regions) may be in conservative and/or non-conservative amino acid substitutions.

Conservative amino acid substitutions can be made in the constant and/or variable regions, including in the CDR or framework regions. A conservative amino acid substitution is a replacement of an amino acid with an amino acid with generally similar properties (e.g., acidic, basic, aromatic, size, positively or negatively charged, polarity, non-polarity) such that the substitution does not substantially negatively alter antibody characteristics (e.g., charge, isoelectric point, affinity, avidity, conformation, solubility) or activity. Typical conservative amino acid substitutions are among the groups of amino acids as follows: glycine (G), alanine (A), valine (V), leucine (L) and isoleucine (I), e.g., A and G; aspartic acid (D) and glutamic acid (E); isoleucine (I), leucine (L), methionine (M) and valine (V); cysteine (C) and methionine (M); alanine (A), serine (S) and threonine (T), e.g., S and T; histidine (H), lysine (K) and arginine (R), e.g., R and K; asparagine (N) and glutamine (Q); phenylalanine (F), tyrosine (Y) and tryptophan (W).

In some embodiments, the antibody is modified in order to further increase its serum half-life. This can be achieved, for example, by mutation of the existing salvage receptor binding epitope present on human or humanized IgG1, IgG2 or IgG4 or by incorporating the epitope into a peptide tag that is then fused to the antibody at either end or in the middle (e.g., by DNA or peptide synthesis).

The antibodies of the invention can be prepared by any conventional means known in the art. For example, polyclonal antibodies can be prepared by immunizing an animal (e.g., a rabbit, rat, mouse, donkey, etc.) by multiple subcutaneous or intraperitoneal injections of the relevant antigen (a purified peptide fragment, full-length recombinant protein, fusion protein, etc.) optionally conjugated to keyhole limpet hemocyanin (KLH), serum albumin, etc. diluted in sterile saline and combined with an adjuvant (e.g., Complete or Incomplete Freund's Adjuvant) to form a stable emulsion. The polyclonal antibody is then recovered from blood, ascites and the like, of an animal so immunized. Collected blood is clotted, and the serum decanted, clarified by centrifugation, and assayed for antibody titer. The polyclonal antibodies can be purified from serum or ascites according to standard methods in the art including affinity chromatography, ion-exchange chromatography, gel electrophoresis, dialysis, etc.

Monoclonal antibodies can be prepared using hybridoma methods, such as those described by Kohler and Milstein (1975) Nature 256:495. Using the hybridoma method, a mouse, hamster, or other appropriate host animal, is immunized as described above to elicit the production by lymphocytes of antibodies that will specifically bind to an immunizing antigen. Lymphocytes can also be immunized in vitro. Following immunization, the lymphocytes are isolated and fused with a suitable myeloma cell line using, for example, polyethylene glycol, to form hybridoma cells that can then be selected away from unfused lymphocytes and myeloma cells. Hybridomas that produce monoclonal antibodies directed specifically against a chosen antigen as determined by immunoprecipitation, immunoblotting, or by an in vitro binding assay (e.g., radioimmunoassay (RIA); enzyme-linked immunosorbent assay (ELISA)) can then be recovered as clonal cell lines and then propagated either in vitro culture using standard methods (Goding, Monoclonal Antibodies: Principles and Practice, Academic Press, 1986) or in vivo as ascites tumors in an animal. The monoclonal antibodies can then be purified from the culture medium or ascites fluid as described for polyclonal antibodies above.

Alternatively, the antigen binding domains of monoclonal antibodies can be isolated using recombinant DNA methods using phage display libraries expressing CDRs of the desired species (e.g., as described in U.S. Pat. Nos. 7,723,270; 7,732,377; 7,662,557; and 7,635,666, in the name of Winter et al., and in McCafferty et al., Nature 348:552-554 (1990); Clackson et al., Nature 352:624-628 (1991); and Marks et al., J. Mol. Biol. 222:581-597 (1991)). The binding domains can also be identified by standard screening methods using either scFv or Fab libraries constructed from naïve or immunized B cell VH and VL cDNA preparations from human, monkey, rodent, rabbit as well as other vertebrate species.

Once antibodies of interest are identified and isolated, the polynucleotide(s) encoding a monoclonal antibody can further be modified in a number of different manners using recombinant DNA technology to generate alternative antibodies. In some embodiments, the constant domains of the light and heavy chains of, for example, a mouse monoclonal antibody can be substituted for those regions of, for example, a human antibody to generate a chimeric antibody. In some embodiments, the constant regions are truncated or removed to generate the desired antibody fragment of a monoclonal antibody. Site-directed or high-density mutagenesis of the variable region can be used to optimize specificity, affinity, etc. of a monoclonal antibody.

Humanized antibodies can be produced using various techniques known in the art. An antibody can be humanized by substituting the CDR of a human antibody with that of a non-human antibody (e.g., mouse, rat, rabbit, hamster, etc. such as the CDR sequences of the murine antibodies disclosed herein) having the desired specificity, affinity, and capability (Jones et al., Nature 321:522-525 (1986); Riechmann et al., Nature 332:323-327 (1988); Verhoeyen et al., Science 239:1534-1536 (1988)). The humanized antibody can be further modified by the substitution of at least one additional residue either in the Fv framework region and/or within the replaced non-human residues to refine and optimize antibody specificity, affinity, and/or capability. Methods of designing humanized antibodies are described in U.S. Pat. Nos. 5,225,539; 5,846,534; 6,569,430; 5,886,152; 5,877,293; 5,821,337; 6,054,297; 6,407,213; 6,639,055; and 6,719,971.

Human antibodies can be directly prepared using various techniques known in the art. Immortalized human B lymphocytes immunized in vitro or isolated from an immunized individual can produce a fully human antibody directed against a target antigen (See, e.g., Cole et al., Monoclonal Antibodies and Cancer Therapy, Alan R. Liss, p. 77 (1985); Boerner et al., J. Immunol. 147(1):86-95 (1991); and U.S. Pat. No. 5,750,373). Human antibodies can also be selected in bacterial hosts like E. coli using a phage display library or cell display library, wherein the display library is introduced into a suitable E. coli strain by transduction or transfection and then can be induced to express human antibodies fragments in one of several possible formats including but not limited to scFv and Fab (Vaughan et al., Nat. Biotech. 14:309-314 (1996); Sheets et al., Proc. Nat'l Acad. Sci. 95:6157-6162 (1998); Hoogenboom et al., J. Mol. Biol. 227:381 (1991); Marks et al., J. Mol. Biol. 222:581 (1991); Daugherty P S et al. 1999)). Full-length human antibodies can be isolated from full-length antibody cell surface display libraries upon transfection into eukaryotic cells, e.g., yeast (Boder ET et al. 1997). Human antibodies can be made in transgenic mice containing human immunoglobulin loci that are capable upon immunization of producing the full repertoire of human antibodies in the absence of endogenous immunoglobulin production. This approach is described in U.S. Pat. Nos. 5,545,807; 5,545,806; 5,569,825; 5,625,126; 5,633,425; and 5,661,016.

In certain embodiments, the antibody is naked (i.e., unconjugated). Regarding therapeutic applications, naked antibodies may mediate cancer cell death via antibody-dependent cellular cytotoxicity (ADCC), which involves cell lysis by effector cells (lymphocytes, NK cells, monocytes, tissue macrophages, granulocytes and eosinophils) that recognize the Fc portion of an antibody. Naked antibodies may cause cancer cell death by activating complement-dependent cytotoxicity (CDC), which involves binding of serum complement to the Fc portion of an antibody and subsequent activation of the complement protein cascade, resulting in cell membrane damage and eventual cell death. Antibodies of different classes and subclasses differ in this respect, as do antibodies of the same subclass but from different species.

In other embodiments, an inventive antibody is conjugated to another moiety, either directly or indirectly. The conjugation may be chemical or biosynthetic. For purposes of therapy, the other agent may be a cytotoxic agent. Applicants' discovery that antibodies of the present invention are internalized by cancer cells makes such Ab-toxin conjugates particularly attractive active anti-cancer agents.

Cytotoxic agents include chemotherapeutic agents, growth inhibitory agents, toxins (e.g., an enzymatically active toxin of bacterial, fungal, plant, or animal origin, or fragments thereof), radioactive isotopes (i.e., a radioconjugate), etc. Chemotherapeutic agents useful in the generation of such immunoconjugates include, for example, methotrexate, adriamicin, doxorubicin, melphalan, mitomycin C, chlorambucil, daunorubicin or other intercalating agents including pro-drug forms. Enzymatically active toxins and fragments thereof that can be used include diphtheria A chain, nonbinding active fragments of diphtheria toxin, exotoxin A chain, ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes. Antibodies can also be conjugated to holo-toxins such as Pseudomonas exotoxin wherein the conjugation procedure inhibits the binding of the holotoxin to its cellular receptor. In some embodiments, the antibodies can be conjugated to radioisotopes, such as ⁹⁰Y, ¹²⁵I, ¹³¹I, ¹²³I, ¹¹¹In, ¹⁰⁵Rh, ¹⁵³SM, ⁶⁷CU, ⁶⁷Ga, ¹⁶⁶Ho, ¹⁷⁷Lu, ¹⁸⁶Re and ¹⁸⁸Re using known chelators or direct labeling. In other embodiments, the antibody is conjugated to a lymphokine such as interferon.

Conjugates of the antibody and cytotoxic agent may be prepared using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutaraldehyde), bis-azido compounds (such as bis(p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). Conjugates of an antibody and one or more small molecule cytotoxic agents, such as a calicheamicin, maytansinoids, a trichothecene, and CC1065, and the derivatives of these compounds that have cytotoxic activity, can also be used. In some embodiments, the antibody can be conjugated with other immunologically active ligands (e.g., antibodies or fragments thereof) wherein the resulting molecule binds to both the neoplastic cell and an effector cell such as a T cell.

The antibodies may also be conjugated to peptides for purposes of enhanced binding and therapeutic activity. One such example is the T15 peptide derived from a monoclonal antibody with self-binding(Kohler et al., Monoclon. Antib. Immunodiag. Immunother. 32(6):425-7 (2013)), which may be attached to the antibody at its nucleotide binding site or installed into the framework region of a recombinant antibody.

Antibodies may be conjugated to a detectable label can be used, for example, to diagnosis disease, to aid in prognosis and to locate OFA/iLRP-expressing cells, in vivo or in vitro. The detectable label produces a measurable signal which is detectable by external means. Detectable labels include an enzyme, a chromophore, a radioisotope, or a substance that emits light by fluorescence, phosphorescence or chemiluminescence. Suitable enzymes include horseradish peroxidase, alkaline phosphatase, β-galactosidase, and acetylcholinesterase. Chromophores include dyes which absorb light in the ultraviolet or visible region, and can be substrates or degradation products of enzyme catalyzed reactions. Suitable fluorescent materials include umbelliferone, fluorescein, fluorescein isothiocyanate, rhodamine, dichlorotriazinylamine fluorescein, dansyl chloride or phycoerythrin. Suitable chemiluminescence materials include luminol, luciferase, luciferin, and aequorin. Suitable radioactive materials for use as detectable labels include ¹²⁵I, ¹³¹I, ³⁵_{S,} ³H and ^{99m}Tc.

Targeting moieties are binding pair members through which other molecules may be attached. Target moieties useful in the present invention include avidin, streptavidin or biotin. For example, biotin may be conjugated to an antibody of the invention, thereby providing a target for another moiety (such as an anti-tumor agent, toxin, detectable label or other antibody) which is conjugated to avidin or streptavidin. Alternatively, the antibody may be conjugated to avidin or streptavidin, thereby providing a target for another moiety which is conjugated to biotin.

The present invention also includes isolated nucleic acid molecules that encode the inventive antibodies or a portion thereof. The nucleic acid molecule encodes an antibody heavy chain, comprising any one of the VH regions or a portion thereof, or which contains the 3 VH CDRs, including any variants thereof, as disclosed herein. Alternatively, the nucleic acid molecule encodes an antibody light chain comprising any one of the VL regions or a portion thereof or which contains the 3 VL CDRs, including any variants thereof as disclosed herein. In certain embodiments, the nucleic acid encodes both a heavy and light chain variable region, or a portion thereof. Examples include the nucleic acid molecules shown in FIGS. 1-4.

The invention also includes recombinant vectors comprising any of the nucleic acid molecules described herein. The vector may comprise a nucleic acid encoding only one antibody chain or a portion thereof (e.g., the heavy or light chain) or a nucleic acid encoding both antibody chains or portions thereof.

Exemplary vectors include plasmids, phagemids, cosmids, viruses and phage particles or other nucleic acid molecules that are capable of replication in a prokaryotic or eukaryotic host. The vectors typically contain a marker to provide a phenotypic trait for selection of transformed hosts such as conferring resistance to antibiotics such as ampicillin or neomycin.

The vector may be an expression vector, wherein the nucleic acid encoding the antibody is operably linked to an expression control sequence. Typical expression vectors contain transcription and translation terminators, initiation sequences, and promoters useful for regulation of the expression of the nucleic acid molecules of the invention. The vectors may also contain genetic expression cassettes containing an independent terminator sequence, sequences permitting replication of the vector in both eukaryotes and prokaryotes, i.e., shuttle vectors and selection markers for both prokaryotic and eukaryotic systems. When the vector contains nucleic acids encoding both a heavy and light chain, or portions thereof, the nucleic acid encoding the heavy chain may be under the same or a separate promoter. The separate promoters may be identical or may be different types of promoters.

Suitable promoters include constitutive promoters and inducible promoters. Representative expression control sequences/promoters include the lac system, the trp system, the tac system, the trc system, major operator and promoter regions of phage lambda, the control region of fd coat protein, the glycolytic promoters of yeast, e.g., the promoter for 3-phosphoglycerate kinase, the promoters of yeast acid phosphatase, e.g., Pho5, the promoters of the yeast alpha mating factors, promoters derived from the human cytomegalovirus, metallothionine promoter, murine mammary tumor virus promoter, Rous sarcoma virus promoter, polyhedrin promoter and promoters derived from polyoma, adenovirus, retrovirus, and simian virus, e.g., the early and late promoters of SV40.

The invention also includes non-human hosts such as cells or organisms containing a nucleic acid molecule or a vector of the invention. By “host” it is meant a non-human unicellular or multicellular organism or a “host cell”, which refers to a cell or population of cells into which a nucleic acid molecule or vector of the invention is introduced. “A population of host cells” refers to a group of cultured cells into which a nucleic acid molecule or vector of the present invention can be introduced and expressed. The host contains a nucleic acid or vector encoding only one chain or portion thereof (e.g., the heavy or light chain variable region); or it may contain a nucleic acid or vector encoding both chains or portions thereof, either on the same or separate nucleic acids and/or vectors.

A host of the present invention may be prokaryotic or eukaryotic. Suitable prokaryotic hosts include E. coli, such as E. coli SG-936, E. coli HB 101, E. coli W3110, E. coli X1776, E. coli X2282, E. coli DHI, and E. coli MRC1, Pseudomonas, Bacillus, such as Bacillus subtilis, and Streptomyces. Suitable eukaryotic cells include yeast and other fungi, insect cells, plant cells, e.g., tobacco cells and cells derived therefrom, human cells, and animal cells, including mammalian cells, such as hybridoma lines, COS cells, NS0 cells and CHO cells. The invention also includes methods of producing an antibody of the present invention, which entails culturing a host cell expressing one or more nucleic acid sequences encoding an antibody of the present invention, and recovering the antibody from the culture medium. In certain embodiments, the antibody is purified by separating it from the culture medium. Antibodies containing more than one chain can be produced by expressing the nucleic acid encoding each chain together in the same host; or as separate chains, and which are assembled before or after recovery from the culture medium.

The antibody proteins produced by a transformed host can be purified according to standard techniques such as chromatography (e.g., ion exchange, affinity and sizing column chromatography), centrifugation, and differential solubility. In addition to affinity purification using protein chromatography, affinity tags such as hexa-histidine, maltose binding domain, influenza coat sequence and/or glutathione-S-transferase may be expressed in-frame to the anti-OFA protein to facilitate purification by passage over an appropriate affinity column. Isolated antibody proteins can also be physically characterized using such techniques as proteolysis, nuclear magnetic resonance and x-ray crystallography.

For example, supernatants from systems which secrete recombinant protein into culture media can be first concentrated using a commercially available protein concentration filter (e.g., an Amicon or Millipore Pellicon ultrafiltration unit). Following the concentration step, the concentrate can be applied to a suitable purification matrix. Alternatively, an anion exchange resin can be employed, for example, a matrix or substrate having pendant diethylaminoethyl (DEAE) groups. The matrices can be acrylamide, agarose, dextran, cellulose or other types commonly employed in protein purification. Alternatively, a cation exchange step can be employed. Suitable cation exchangers include various insoluble matrices comprising sulfopropyl or carboxymethyl groups. Further degree of purification may be achieved by one or more reverse-phase high performance liquid chromatography (RP-HPLC) steps employing hydrophobic RP-HPLC media, e.g., silica gel having pendant methyl or other aliphatic groups. Combinations of two or more purification steps may be employed.

Recombinant antibody protein produced in bacterial culture can be isolated, for example, by initial extraction from cell pellets, followed by one or more concentration, salting-out, aqueous ion exchange or size exclusion chromatography steps. HPLC can be employed for final purification steps. Microbial cells employed in expression of a recombinant antibody protein can be disrupted by any convenient method, including freeze-thaw cycling, sonication, mechanical disruption, or use of cell lysing agents.

This invention further provides a pharmaceutical composition comprising an antibody of this invention and a pharmaceutically acceptable vehicle. The composition may be formulated for any medically acceptable and efficacious route of administration, including oral, parenteral (e.g., intravenous, intraperitoneal, infusion, intraarterial, intramuscular, subcutaneous), topical (which includes transmucosal and transdermal), and pulmonary administration. The composition may be formulated as an immediate, controlled, extended or delayed release composition. The pharmaceutical composition of the invention may be formulated in a variety of ways, including for example, liquid, semi-solid and solid dosage forms, such as liquid solutions (e.g., injectable and infusible solutions), dispersions or suspensions, tablets, pills, powders, liposomes and suppositories.

Pharmaceutically acceptable vehicles include nontoxic buffers such as phosphate, citrate, and other organic acids; salts such as sodium chloride; antioxidants including ascorbic acid and methionine; preservatives (e.g., octadecyldimethylbenzyl ammonium chloride; hexamethonium chloride; benzalkonium chloride; benzethonium chloride; phenol, butyl or benzyl alcohol; alkyl parabens, such as methyl or propyl paraben; catechol; resorcinol; cyclohexanol; 3-pentanol; and m-cresol); low molecular weight polypeptides (e.g., less than about 10 amino acid residues); proteins such as serum albumin, gelatin, or immunoglobulins; hydrophilic polymers such as polyvinylpyrrolidone; amino acids such as glycine, glutamine, asparagine, histidine, arginine, or lysine; carbohydrates such as monosaccharides, disaccharides, glucose, mannose, or dextrins; chelating agents such as EDTA; sugars such as sucrose, mannitol, trehalose or sorbitol; salt-forming counterions such as sodium; metal complexes (e.g., Zn-protein complexes); and non-ionic surfactants such as TWEEN or polyethylene glycol (PEG).

More particularly, preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. In the subject invention, pharmaceutically acceptable carriers include, but are not limited to, 0.01-0.1M and preferably 0.05M phosphate buffer or 0.8% saline. Other common parenteral vehicles include sodium phosphate solutions, Ringer's dextrose, dextrose and sodium chloride, lactated Ringer's, or fixed oils. Intravenous vehicles include fluid and nutrient replenishers, electrolyte replenishers, such as those based on Ringer's dextrose, and the like. Preservatives and other additives may also be present such as for example, antimicrobials, antioxidants, chelating agents, and inert gases and the like. The composition should be sterile and fluid for purposes of ease of syringability.

The pharmaceutical composition can be formulated in a unit dosage form. Such formulations include tablets, pills, capsules, powders, granules, solutions or suspensions in water or non-aqueous media, or suppositories for oral, parenteral, or rectal administration or for administration by inhalation. In solid compositions such as tablets the antibody is mixed with a vehicle e.g., corn starch, lactose, sucrose, sorbitol, talc, stearic acid, magnesium stearate, dicalcium phosphate or gums, and other diluents (e.g., water) to form a solid preformulation composition. The solid preformulation composition is then subdivided into unit dosage forms of the type described above. The tablets, pills, etc. can be coated or otherwise compounded to provide a dosage form affording the advantage of prolonged action. For example, the tablet or pill can comprise an inner composition covered by an outer component. Furthermore, the two components can be separated by an enteric layer that serves to resist disintegration and permits the inner component to pass intact through the stomach or to be delayed in release. A variety of materials can be used for such enteric layers or coatings, such materials including a number of polymeric acids and mixtures of polymeric acids with such materials as shellac, cetyl alcohol and cellulose acetate.

Sterile injectable solutions can be prepared by incorporating the antibody and the vehicle, in the required amount followed by filtered sterilization. Generally, dispersions are prepared by incorporating the antibody into a sterile vehicle including a basic dispersion medium. In the case of sterile powders for the preparation of sterile injectable solutions, one method of preparation is vacuum drying and freeze-drying, which yields a powder of the antibody from a previously sterile-filtered solution thereof. The preparations for injections are processed, filled into containers such as ampoules, bags, bottles, syringes or vials, and sealed under aseptic conditions according to methods known in the art.

The inventive antibodies may be complexed with liposomes (Epstein, et al., 1985, Proc. Natl. Acad. Sci. USA 82:3688; Hwang, et al., 1980, Proc. Natl. Acad. Sci. USA 77:4030; and U.S. Pat. Nos. 4,485,045 and 4,544,545). Liposomes with enhanced circulation time are disclosed in U.S. Pat. No. 5,013,556. Some liposomes can be generated by the reverse phase evaporation with a lipid composition comprising phosphatidylcholine, cholesterol, and PEG-derivatized phosphatidylethanolamine (PEG-PE). Liposomes are extruded through filters of defined pore size to yield liposomes with the desired diameter.

The antibodies can also be entrapped in microcapsules. Such microcapsules are prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions as described in Remington, The Science and Practice of Pharmacy 20th Ed. Mack Publishing (2000).

In addition sustained-release preparations can be prepared. Suitable examples of sustained-release preparations include semipermeable matrices of solid hydrophobic polymers containing the antibody, which matrices are in the form of shaped articles (e.g., films, or microcapsules). Examples of sustained-release matrices include polyesters, hydrogels such as poly(2-hydroxyethyl-methacrylate) or poly(vinylalcohol), polylactides (U.S. Pat. No. 3,773,919), copolymers of L-glutamic acid and 7-ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), sucrose acetate isobutyrate, and poly-D-(−)-3-hydroxybutyric acid.

Further, the preparations may be packaged and sold in the form of a kit. If the antibody is freeze-dried (for purposes of prolonged storage stability), the kit may also contain a solvent for reconstituting the antibody. Such articles of manufacture may also have labels or package inserts indicating that the associated compositions are useful for therapeutic or diagnostic purposes. Thus, in some embodiments, the kit also contains additional components necessary to conduct a detection assay, in which case the antibody may be conjugated to a detectable label.

The invention further provides methods of treating cancer, which entails administering to a subject in need thereof a therapeutically effective amount of an antibody of the invention. As used herein, the term “cancer” refers to or describes the physiological condition in mammals in which a population of cells is characterized by unregulated cell growth. As used herein, the term “subject” refers to any animal (e.g., a mammal), including, but not limited to humans, non-human primates, domestic animals (e.g., dogs and cats), rodents, and the like, which is to be the recipient of a particular treatment or protocol describe herein. Typically, the terms “subject” and “patient” are used interchangeably herein in reference to a subject that is a human subject.

As used herein, the terms “treat” and “treatment” refer to therapeutic treatment, including prophylactic or preventative measures, wherein the object is to prevent or slow down (lessen) an undesired physiological change associated with cancer. Beneficial or desired clinical results may include alleviation of symptoms, diminishment of the extent of a disease or condition, stabilization of a disease or condition (i.e., where the disease or condition does not worsen), delay or slowing of the progression of a disease or condition, amelioration or palliation of the disease or condition, and remission (whether partial or total) of the disease or condition, whether detectable or undetectable. “Treatment” can also mean prolonging survival as compared to expected survival if not receiving treatment. Those in need of treatment include those already with the disease or condition as well as those prone to having the disease or condition or those in which the disease or condition is to be prevented.

Cancers to be treated include primary tumors and secondary or metastatic tumors (including those metastasized from lung, breast, or prostate), as well as recurrent or refractory tumors. Recurrent tumors encompass tumors that appear to be inhibited by treatment with such agents, but recur up to five years, sometimes up to ten years or longer after treatment is discontinued. Refractory tumors are tumors that have failed to respond or are resistant to treatment with one or more conventional therapies for the particular tumor type. Refractory tumors include those that are hormone-refractory (e.g., androgen-independent prostate cancer; or hormone-refractory breast cancer, such as breast cancer that is refractory to tamoxifen); those that are refractory to treatment with one or more chemotherapeutic agents; those that are refractory to radiation; and those that are refractory to combinations of chemotherapy and radiation, chemotherapy and hormone therapy, or hormone therapy and radiation.

Therapy may be “first-line”, i.e., as an initial treatment in patients who have undergone no prior anti-cancer treatment regimens, either alone or in combination with other treatments; or “second-line”, as a treatment in patients who have undergone a prior anti-cancer treatment regimen, either alone or in combination with other treatments; or as “third-line”, “fourth-line”, etc. treatments, either alone or in combination with other treatments.

Therapy may also be given to patients who have had previous treatments which have been partially successful but are intolerant to the particular treatment. Therapy may also be given as an adjuvant treatment, i.e., to prevent reoccurrence of cancer in patients with no currently detectable disease or after surgical removal of tumor.

Types of cancers to be treated with the antibodies of the invention include carcinomas, sarcomas, benign and malignant tumors, and malignancies. Adult tumors/cancers and pediatric tumors/cancers are included. The cancers may be vascularized, or not yet substantially vascularized, or non-vascularized tumors. The cancers may be characterized by non-solid tumors (e.g., hematopoietic cancers such as leukemias (e.g., ALL, AML, CLL, and CML) and lymphomas (Hodgkins and non-Hodgkins) or solid tumors.

Examples of cancers characterized by solid tumors which may be treated in accordance with the present invention include breast (including HER2+ and metastatic), colorectal, colon, renal, rectal, pancreatic, prostate, stomach, gastrointestinal, gastric, stomach, esophageal, bile duct, lung (including small cell and non-small cell lung tumors, adenocarcinoma of the lung and squamous carcinoma of the lung), liver, lymphoma, epidermoid tumors, squamous tumors such as head and neck tumors, epithelial squamous cell cancer, thyroid, cervical, ovarian, neuroendocrine tumors of the digestive system, neuroendocrine tumors, pheochromacytomas, cancer of the peritoneum, hepatocellular cancer, hepatoblastoma, HPCR, glioblastoma, bladder cancer, hepatoma, endometrial or uterine carcinoma, salivary gland carcinoma, kidney or renal cancer, bone cancer, soft tissue sarcoma (including embryonal and alveolar rhabdomyosarcoma, GIST, alveolar soft part sarcoma and clear cell sarcoma), cholangiocarcinoma, bile cancer, gallbladder carcinoma, myeloma, vulval cancer, hepatic carcinoma, anal carcinoma, penile carcinoma, retinal, androgen-dependent tumors, androgen-independent tumors, Kaposi's sarcoma, synovial sarcoma, vasoactive intestinal peptide secreting tumor, CNS neoplasms, neuroblastomas, capillary hemangioblastomas, meningiomas, and cerebral metastases, melanoma, rhabdomyosarcoma, glioblastoma, including glioblastoma multiforme, EMB, RMS, ALV, medulloblastoma, ependymoma, Wilm's cancer, Ewing's cancer, osteosarcoma, PNT, rhabdoid, rhabdomyosarcoma, retinoblastoma, adrenal cortical cancer, adrenal cancer, and leiomyosarcoma.

In some embodiments, the treatment methods of the present invention entail targeting (directly or indirectly) of cancer stem cells. The evolution of a normal cell into a fully transformed one requires the deregulation of multiple cellular processes. According to classical models of carcinogenesis, these events can occur in any cell. The “cancer stem cell hypothesis” holds that the preferential targets of oncogenic transformation are tissue stem or early progenitor cells that have acquired self-renewal potential. See, e.g., Bonnet et al., Nat. Med. 3:730-737 (1997); Glinsky et al. Stem Cell Rev. 3:79-93 (2007); Jaiswal et al., Proc. Natl. Acad. Sci. U.S.A 100:10002-10007 (2003); and Krivtsov et al. Nature 442:818-822 (2006). These “tumor-initiating cells” or “cancer stem cells” (CSC), in turn, are characterized by their ability to undergo self-renewal, a process that drives tumorigenesis and differentiation which contributes to tumor cellular heterogeneity.

The relative abundance of cancer stem cells in a total population of cancer cells may be influenced by the course of treatment. For instance, it is reported that treatment regimens that cause increased levels of various cytokines such as IL-6 and IL-8 cause an increase in cancer stem cell number. The inventive antibodies will effectively target OFA/iLRP that may be present on cancer stem cells. To the extent it is desired to enhance targeting of cancer stem cells in any given course of treatment, the regimen may be supplemented, e.g., by administering an IL8-CXCR1 pathway inhibitor (e.g., an anti-CXCR1 antibody), a IL-6 pathway inhibitor (e.g., anti-IL-6R antibody such as Tocilizumab), alone or in combination with an additional chemotherapeutic agent, such that non-tumorigenic and tumorigenic cancer cells in a subject are killed. See, e.g., U.S. Patent Application Publications 20130142785 A1 and 20100136031 A1.

The antibody may be administered only once, or it may be administered over a series of treatments lasting from several days to several months or until cure, remission, or a diminution in disease state (e.g., reduction in tumor size). For multiple dosages, the antibody may be, for example, administered three times a day, twice a day, once a day, once every two days, twice a week, weekly, once every two weeks, once every three weeks, or monthly.

The antibodies of the invention are administered in a “therapeutically effective amount”. A “therapeutically effective amount” refers to an amount effective, at dosages and for periods of time necessary, to “treat” the cancer as that term is used herein. For example, the therapeutically effective amount of the drug can reduce the number of cancer cells, reduce the tumor size; inhibit or stop cancer cell infiltration into peripheral organs including, for example, the spread of cancer into soft tissue and bone, inhibit and stop tumor metastasis, inhibit and stop tumor growth, relieve or reduce one or more of the symptoms associated with the cancer, reduce morbidity and mortality, improve quality of life, or a combination thereof. A therapeutically effective amount of the antibody may vary according to factors such as the disease state, age, sex, and weight and overall health of the individual, any previous treatment, patient's clinical history, and the ability of the antibody or antibody fragment to elicit a desired response in the subject. A therapeutically effective amount is also one in which any toxic or detrimental effects of the antibody or antibody portion are outweighed by the therapeutically beneficial effects. Depending on the mode of administration, a therapeutically effective amount of an antibody of the invention may vary from about 0.000125 to about 100 mg per kg of body weight, and in some embodiments about 1 to about 30, and in some other embodiments, from about 2 to about 6 mg/kg, per week. In some embodiments, an initial dose of the antibody may be administered, e.g., in amounts ranging from about 2 to about 20 mg/kg, and in some embodiments from about 3-12 mg/kg.

The methods of treatment described herein can be used to treat any subject in need thereof, e.g., mammals, including primates, such as monkeys and humans, horses, cows, cats, dogs, rabbits, and rodents such as rats and mice. In one embodiment, the mammal to be treated is human.

More than one OFA/iLRP antibody may be administered, either incorporated into the same composition or administered as separate compositions. For example, as shown in the working examples, administration of the monoclonal antibody BV-27 may potentiate the therapeutic activity of the monoclonal antibody BV-15.

The antibody may be administered alone (monotherapy), or in combination with one or more therapeutically effective active agents (e.g., anti-cancer agents) or treatments (combination therapy). The other therapeutically effective agent may be conjugated to the antibody, incorporated into the same composition as the antibody, or may be administered as a separate composition. The other therapeutically effective agent or treatment may be administered prior to, during and/or after the administration of the antibody. The other therapeutically effective agent may be administered to augment the therapeutic effect of the antibody, or to diminish the negative side effects of the antibody.

Other anti-cancer therapeutically effective agents/treatments include surgery, radiation chemotherapeutic agents, cytokines, chemokines and biological agents such as antibodies to other targets, and enzymes. As disclosed above, in some embodiments, combination therapy may be achieved by conjugating the antibody to a cytotoxic agent (e.g., including FDA-approved drugs and those which have failed in clinical testing due to toxicity and for which conjugation reduces said toxicity).

The anti-neoplastic agent also includes radiation. When the anti-neoplastic agent is radiation, the source of the radiation can be either external (external beam radiation therapy—EBRT) or internal (brachytherapy—BT) to the patient being treated. The dose of anti-neoplastic agent administered depends on numerous factors, including, for example, the type of agent, the type and severity of tumor being treated and the route of administration of the agent. It should be emphasized, however, that the present invention is not limited to any particular dose. Radiation may also be used in conjunction with other antineoplastic agents.

Examples of chemotherapeutic agents (which are typically small molecules) include topoisomerase inhibitors (e.g., inhibitors of topoisomerase I or topoisomerase II. Topoisomerase I inhibitors such as irinotecan (CPT-II), aminocamptothecin, camptothecin, DX-8951f, topotecan. Topoisomerase II inhibitors include etoposide (VP-16), and teniposide (VM-26)), cyclophosphamide, Thiotepa, bysulfan, melphalan, dacarbazine, cytosine arabinoside, cyclophosphamide, actinomycin-D, methotrexate, gemcitabine, oxyplatin, fluorouracil (5-FU), leucourin (LU), cisplatin, irinotecan (CPT-II), paclitaxel, docetaxel, vinblastine, epothilone, carboplatin, pegylated adriamycin, anthracyclines (e.g., daunomycin and doxorubicin), vindesine, neocarzinostatin, cis-platinum, chlorambucil, cytosine arabinoside, 5-fluorouridine, and calicheamicin.

A chemotherapeutic agent may be administered as a prodrug. The term “prodrug” refers to a precursor or derivative form of a pharmaceutically active substance that is less cytotoxic to tumor cells compared to the parent drug and is capable of being enzymatically activated or converted into the more active parent form. Examples of prodrugs include phosphate-containing prodrugs, thiophosphate-containing prodrugs, sulfate-containing prodrugs, peptide-containing prodrugs, D-amino acid-modified prodrugs, glycosylated prodrugs, beta-lactam-containing prodrugs, optionally substituted phenoxyacetamide-containing prodrugs or optionally substituted phenylacetamide-containing prodrugs, 5-fluorocytosine and other 5-fluorouridine prodrugs which can be converted into the more active cytotoxic free drug.

“Cytokines” refer to proteins and derivatives thereof released by one cell population that act on another cell as intercellular mediators. Examples of such cytokines are lymphokines, monokines, and traditional polypeptide hormones. Included among the cytokines are growth hormones such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; fibroblast growth factor; prolactin; placental lactogen; tumor necrosis factor-α and TNF-β; mullerian-inhibiting substance; mouse gonadotropin-associated peptide; inhibin; activin; vascular endothelial growth factor; thrombopoietin (TPO); nerve growth factors such as NGF-beta; platelet-growth factor; transforming growth factors (TGFs) such as TGF-alpha and TGF-beta; insulin-like growth factor-I and ILGF-II; erythropoietin (EPO); osteoinductive factors; interferons such as interferon-α, -β, and -γ; colony stimulating factors (CSFs) such as macrophage-CSF (M-CSF); granulocyte-macrophage-CSF (GM-CSF); and granulocyte-CSF (G-CSF); interleukins (ILs) such as IL-1, IL-1 alpha, IL-2, IL-3, IL-4, IL-5, IL-6, IL-7, IL-8, IL-9, IL-10, IL-11, IL-12; IL-15, and other polypeptide factors including LIF and kit ligand (KL).

Chemokines include MIP-1α, MIP-1β, RANTES, SDF-1, MCP-1, MCP-2, MCP-3, MCP-4, eotaxin, eotaxin-2, I-309/TCA3, ATAC, HCC-1, HCC-2, HCC-3, LARC/MIP-3α, PARC, TARC, CKβ, CKβ6, CKβ7, CKβ8, CKβ9, CKβ11, CKβ12, C10, IL-8, GROα, GROβ, ENA-78, GCP-2, PBP/CTAβIIIβ-TG/NAP-2, MIG, PBSF/SDF-1, and lymphotactin.

Antibodies to other targets include antibodies to TrkB, TrkC, CD19, CD20, CD33, CD44, CD45, CD46, CD59, EGFR, EGF, VEGF, VEGFR-1, VEGFR-2, VEGFR-3, PDGFR, PDGF, IGFR, IGF, NGFR, NGF, FGFR, FGF, RON, gp75, Flt-3, Fas, AFP, PDFG, Natural Killer cells, CA 125, CEA, T cell receptor alpha/beta, GD₂, GD₃, GM1, GM2, Her-2/Neu, Ep-CAM (KSA), endothelin receptor, IL-2 receptor, IL-6 receptor, (e.g., Tocilizumab) IL-8 receptor (e.g., anti-CXCR1 antibodies), Lewis-Y, Lewis-X (CD 15), melanoma-associated proteoglycan MCSP, PSA, cadherin, and the transferrin receptor.

For example, an antibody against EGFR, such as Erbitux® (cetuximab), may also be administered, particularly when treating colon or head and neck cancer. Other antibodies for combination use include Herceptin (trastuzumab) (against breast cancer cells that express HER2, or HER2 expression on other cancer cells) and Avastin® (bevacizumab) (an antibody that inhibits angiogenesis). Other antibodies for combination are antibodies which specifically bind human insulin-like growth factor-1 (IGFR). See, e.g., WO 2005/016970 and U.S. Pat. No. 7,241,444.

Enzymatically active toxin or fragments thereof that can be used include diphtheria A chain, nonbinding active fragments of diphtheria toxin, exotoxin A chain or holo-toxin (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes.

Representative examples of other therapeutic agents (which are not necessarily anti-cancer agents) include antibiotics that target gram-negative bacteria, including broad spectrum antimicrobials that cover both gram-positive and gram-negative organisms, including quinolones (e.g., Baytril, ciprofloxacin), cephalosporins (e.g., cefepime, ceftazidine) and aminoglycosides (e.g., gentamicin, amikacin).

The administration of the antibodies with other agents and/or treatments may occur simultaneously, or separately, via the same or different route, at the same or different times. Dosage regimens may be adjusted to provide the optimum desired response (e.g., a therapeutic or prophylactic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic or prophylactic effect to be achieved, and (b) the limitations inherent in the art of compounding such an active compound for the treatment of sensitivity in individuals.

The present invention further provides the method of using the present antibodies diagnostically. As used herein, “diagnosing” or “diagnostic” refers to any information that is useful in determining whether a subject has cancer or in classifying the cancer into a category having significance with regards to prognosis, the selection of treatment or likely response to treatment (including monotherapy and combination therapy), and the monitoring of treatment (wherein a decrease in the relative amounts of OFA/iLRP would indicate that therapy is effective, and an unchanged or increase in the relative amounts of OFA/iLRP would indicate that therapy is ineffective). The diagnostic methods are applicable to humans as well as to animals such as domestic animals (e.g., dogs and cats).

Diagnostic information may be obtained via a biopsy or biopsy tissue, which as used herein, refers to a sample of tissue or fluid, e.g., a blood sample (or serum obtained therefrom), that is removed from a subject for the purpose of detecting the presence and relative amount of OFA. Detection of OFA/iLRP in the sample can be accomplished using one or more of the antibodies of the present invention, which may be detectably labeled, and standard analytic biochemical methods such as, for example, ELISA, FACS (e.g., particularly for hematopoietic cancers such as leukemia and lymphoma), immunohistochemistry (e.g., particularly for solid tumors and pelleted tumor cells), immunoprecipitation and protein microarrays. Labeling systems are known in the art and typically entail use of chromogenic, fluorescent and chemiluminescent labels or agents. Analysis of the results may be semi-quantitative or quantitative.

In some embodiments, an ELISA (also known as an antibody sandwich assay) may be performed following standard techniques as follows. Any of the inventive antibodies may be used as the capture antibody. The antibody is disposed on (e.g., coated onto) a solid support, e.g., a Nunc Star Immunosorb plate. The solid support may then be washed at least once (e.g., with water and/or a buffer such as PBS-t), followed by a standard blocking buffer, and then at least one more wash. The solid support is then brought into contact with the fluid sample or a lysated extract from a tissue sample under conditions to allow an antibody-antigen complex to form (e.g., incubating from 1 hour to about 24 hours at a temperature from about 4° C. to about room temperature). The support may then be washed at least once (e.g., with a buffer such as PBS-t). To detect the complexation between the capture antibody and the OFA/iLRP that may be present in the sample, a secondary or “detection” antibody is applied to the solid support (e.g., diluted in blocking buffer) under conditions to allow complexation between the secondary antibody and the antigen (e.g., at room temperature for at least one hour). The secondary antibody may be an inventive antibody that binds a different epitope than the capture antibody, or it may be a non-inventive antibody that has specificity for either the immature or the mature form of OFA or LRP, respectively, the proviso being that the capture antibody and the detection antibody should not cross-inhibit each other. Thus, if BV-15 is used as a capture antibody, the detection antibody is not BV-27. The optimum concentration of capture and detection antibody is determined using standard techniques such as the “criss-cross” method of dilutions. The detection antibody is conjugated to a (directly or indirectly) chromogenic label such as biotin, in which case the biotin conjugate is detected using Streptavidin/Horseradish Peroxidase (HRP) or the equivalent. The streptavidin may be diluted in an appropriate block and incubated for 30 minutes at room temperature. Other detectable labels suitable for use in ELISA include fluorescent labels and chemiluminescent labels. The support may then be washed and the label (e.g., HRP enzymatic conjugate on the streptavidin) is detected using following standard protocols such as a chromogenic system (the SIGMA FAST™ OPD system), a fluorescent system or a chemiluminescent system. The amount of OFA/iLRP present in a fluid sample is then read on an ELISA plate reader (e.g., SpectraMax 384 or the equivalent). The concentration of OFA/iLRP may then be back-calculated (e.g., by using the standard curve generated from purified OFA/iLRP and multiplied by the dilution factor following standard curve fitting methods), and then compared to a control (generated from tissue samples obtained from healthy subjects).

The invention will now be described in terms of the following, non-limiting examples.

### EXAMPLE 1

### Determining Antibody Affinity

Assessment of MAb affinity by ELISA against the immunizing antigen (rOFA). High antibody affinity is thought to be important for binding to target antigen. More recent data suggests that moderate affinity may be better than high affinity for penetration into tumors and therapeutic activity, Rudnick et al., 2009 (1). Moreover, from a practical consideration, screening for tumor and normal tissue reactivity could be misleading if an antibody is of too low affinity.

Therefore antibody affinity (Kd M) for each of BV-15, BV-27, BV-12 and BV-06, as well as non-inventive antibodies BV-19, 2C6 and 3G7 was determined utilizing the approach of Beatty et al., (1987) (2). Briefly, antigen (rOFA) was coated onto the solid phase of micro-titer plates (Immulon IV, Nunc) in carbonate buffer (Warner et. al. 1986) (3) at 3 sequential Log₂ concentrations (0.5, 0.25 and 0.125 μg/ml) and allowed to bind overnight at 4° C. After washing and blocking the wells with 0.5% BSA, antibody solutions of individual clones were added at 12 sequential Log₂ concentrations in diluent and allowed to react for 60 minutes at room temperature on a rotary platform shaker. Antibody binding to OFA was followed with biotinylated anti-mouse IgG and streptavidin-HRP. Bound anti-OFA antibodies (biotinylated anti-mouse IgG/streptavidin-HRP) were quantified utilizing TMB substrate addition and subsequent color development.

Data were plotted on semi-log paper with optical densities plotted on the Y-axis (Linear) and antibody concentrations plotted on the X-axis (Log₁₀). Antibody concentrations at the lower levels were at a molar deficit and at the upper levels were at molar excess, relative to OFA concentrations, producing a curve that is sigmoid in shape. The high OFA concentration curve (0.5 μg/ml) was set as the 100% binding curve; the 0.25 μg/ml curve corresponded to the 50% binding curve and 0.125 μg/ml was set as the 25% binding curve. The point at which a line drawn from the upper plateau of the 50% binding curve bisects the 100% binding curve and then dropped from this point to the antibody concentration is referred to as Ab. The same is then done with the 25% binding curve bisecting the 50% binding curve and the subsequent value is set as Ab′. Utilizing the Molar concentration of Ab and Ab′ in the equation: 1/2(2[Ab′]ₜ−[Ab]ₜ) of Beatty et.al., we then determined the Kd M value.

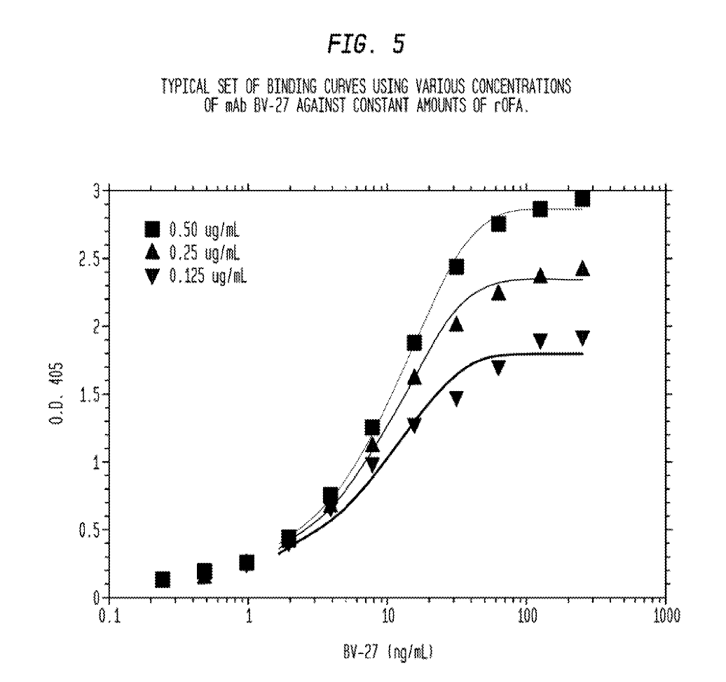

The data are shown in Table 1. As part of the studies, soluble rOFA was able to compete with the bound substrate for binding with each of the antibodies. FIG. 5 shows a typical set of binding curves using MAb BV-27.

TABLE 1
Affinity versus rOFA

| Antibody | Antigen | Kd M |
|---|---|---|
| BV-15 (IgG2a) | rOFA | 7.87 ± 0.91 × 10⁻¹¹ |
| BV-27 (IgG2a) | rOFA | 7.63 ± 1.66 × 10⁻¹¹ |
| BV-06 (IgG2a) | rOFA | 6.32 ± 1.11 × 10⁻¹¹ |
| BV-12 (IgG2a) | rOFA | 2.98 ± 0.82 × 10⁻¹⁰ |
| BV-19 (IgG2b) + | rOFA | 1.72 ± 1.47 × 10⁻¹⁰ |
| 2C6 (IgG2b) | * | 6.29 ± 2.54 × 10⁻⁸ |
| 3G7 (IgG1) | * | 1.48 ± 1.25 × 10⁻⁷ |

* 2C6 and 3G7 are described and claimed in U.S. Application 12/732,880, filed Mar. 26, 2010, entitled ONCOFETAL ANTIGEN/IMMATURE LAMININ RECEPTOR ANTIBODIES FOR DIAGNOSTIC AND CLINICAL APPLICATIONS. The antibodies of that invention were generated to particular synthetic peptide sequences derived from the C-terminal portion of OFA and are included herein and throughout this application as references and in comparison to the above claimed antibodies (BV-15, BV-27, BV-06, and BV-12.
+ BV-19 is included as a reference and comparison to the antibodies of this invention. Although generated by immunizing with OFA, this antibody recognizes Mature Laminin Receptor Protein.
Kd values for 2C6, 3G7 and BV-19 were also calculated according to the method of Beatty et. al (1987).

### Citations:

(1) Stephen I. Rudnick and Gregory P. Adams (2009). Affinity and Avidity in Antibody-Based Tumor Targeting, Cancer Biotherapy & Radiopharmaceuticals, Cancer Biother. Radiopharm. 2009 April; 24(2): 155-161.

(2) Beatty J. D., Beatty B. G., Vlahos W. G., Hill L. R. (1987). Method of analysis of non-competitive enzyme immunoassays for antibody quantification. J. Immunol. Methods 100(1-2):161-172; Beatty J. D., Beatty B. G., Vlahos W. G., Measurement of Monoclonal Antibody Affinity by Non-Competitive Enzyme Immunoassay, J. Immunol. Methods 100(1-2):173-179.

(3) Warner, R. L., Ram, B. P., Hart, L. P., and Pestka, J. J. (1986). Screening for zearalenone in corn by competitive direct enzyme-linked immunosorbent assay. J. Agric. Food Chem. 34(4):714-717.

DOI: 10.1021/jf00070a031 GraphPad Prism version 4.00 for Windows, GraphPad Software, San Diego Calif. USA

### EXAMPLE 2

### Antibody Affinity Versus Native OFA

BV-15, BV-27, BV-06, and BV-12 antibodies were quantitatively assessed against cell surface, native OFA. In these studies, antibody affinity as well as antigen density were measured

Cells. Two immortalized human myelogenous leukemia lines (HL-60 and K562) were used for these studies.

Flow cytometry. Flow cytometric analysis was done using a BD LSRII cytometer (Serial #: H47300011; HTS Serial #: U30100321) and data were analyzed using BD FacsDiva Software.

Antigen density. A commercially-available assay kit (QIFIKIT; Dako) was used to quantify antibody binding capacity. The assay kit contains polystyrene beads (10 μm in diameter) coated with different (but precisely known) amounts of a mouse IgG antibody directed against CD5 antigen. In the initial studies, HL60 cells were labeled with MAb BV-27 in the standard way, using a saturating amount of antibody (7.5 μg per reaction). A control mouse IgG2a was used as control. Then, cells with bound antibody or IgG control and polystyrene beads containing bound CD5 were labeled with the secondary antibody in an identical manner under identical conditions. Cells and beads were analyzed together by flow cytometry and the amount of bound antibody was determined by direct comparison with the standard curve generated with the styrene beads. Following initial studies with BV-27, 3 other inventive antibodies were assessed in the same manner.

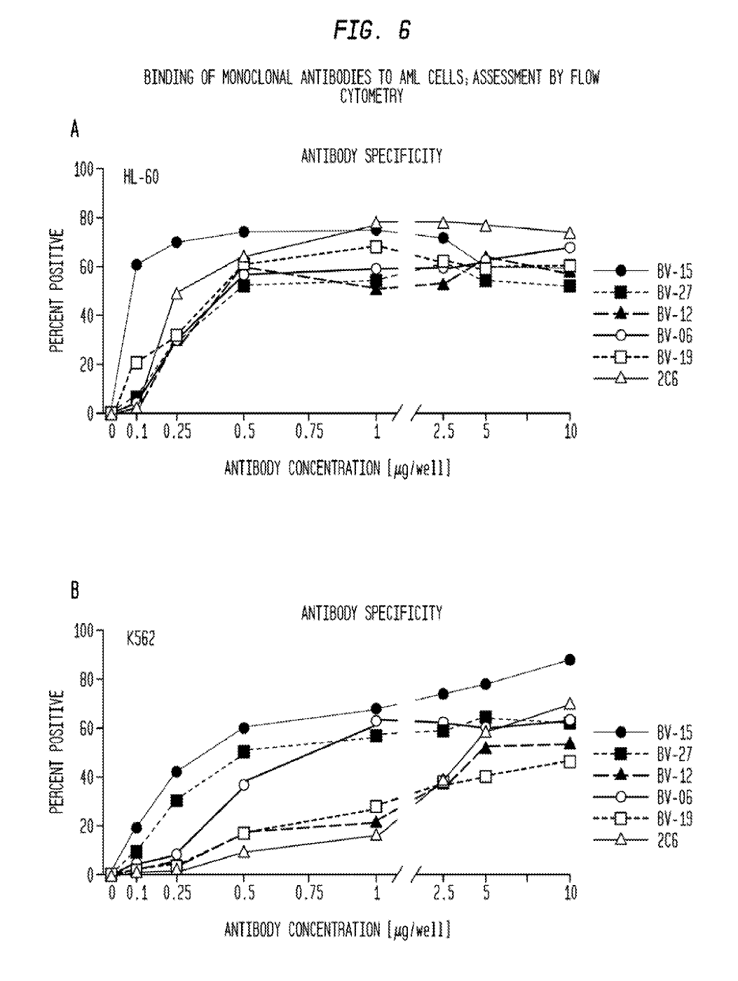

Results. Results from direct binding studies. FIGS. 6A-B show antibody-binding curves for HL-60 and K562 cells with the panel of monoclonal antibodies. These figures show that both cell lines express epitopes recognized by each of the antibodies in the panel raised against rOFA. It can also be seen from the binding curves that antibody affinities for cell surface OFA/iLRP varied within a small range. The half-maximal binding values for each of the antibodies for the two cell types are presented in Table 2.

TABLE 2
Half-maximal Binding values for MAb
binding to HL-60 and K562 cells

| Antibody | K562 ([Ab] in M) | HL60 |
|---|---|---|
| BV-15 | 2.38 × 10⁻⁹ | 4.40 × 10⁻¹⁰ |
| BV-27 | 2.81 × 10⁻⁹ | 3.02 × 10⁻⁹ |
| BV-12 | 2.56 × 10⁻⁸ | 2.50 × 10⁻⁹ |
| BV-06 | 5.00 × 10⁻⁹ | 2.63 × 10⁻⁹ |
| BV-02 | 3.12 × 10⁻⁹ | not done |
| BV-19 | >6.25 × 10⁻⁸ | 2.75 × 10⁻⁹ |
| 2C6 | 2.38 × 10⁻⁹ | 1.75 × 10⁻⁹ |

These values were calculated based on flow cytometric analysis. Where the value is greater than 6.25×10⁻⁸ M, this indicates we did not reach 50% binding with 10 μg of antibody per ml. In contrast to the studies with rOFA, 2C6 expressed high affinity against native OFA while low affinity against rOFA. All of the antibodies of this invention had relatively high affinity to both targets.

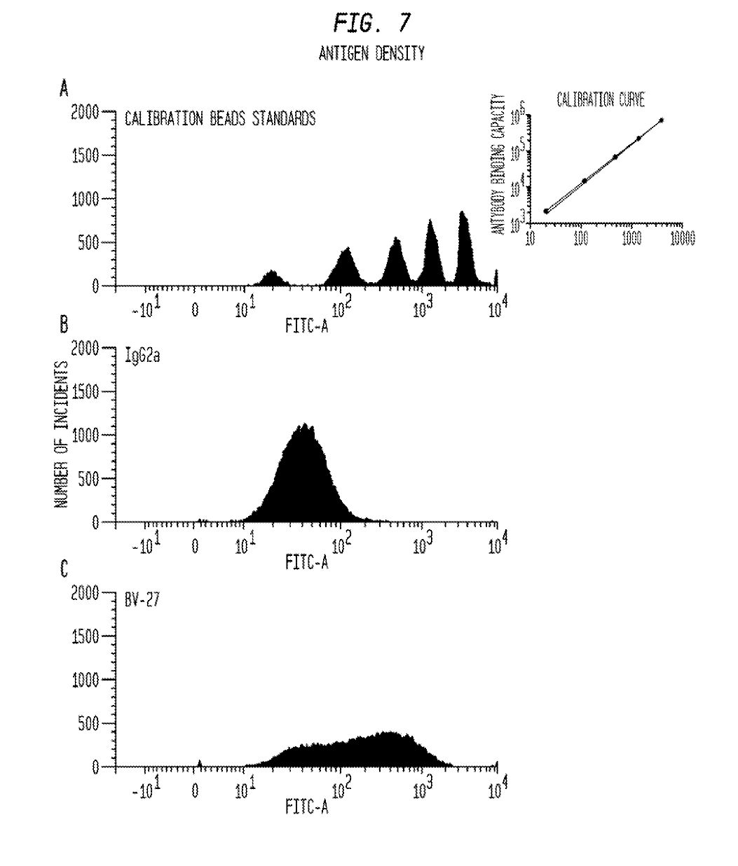

Antigen density. FIGS. 7A-C show histograms from the styrene bead method for determining antigen density on HL-60 cells. For the studies depicted in the histogram, monoclonal antibody BV-27 was used. The antibody was used at a saturating concentration (10 micrograms per reaction). By comparison with the calibration curve shown in the insert, the number of binding sites per cell was calculated to be 7.53×10⁴ sites per cell. When K562 cells were examined in the same assay with monoclonal antibody BV-27, a value of 5.4×10⁴ sites per cell was attained. With K562 cells, three of the other antibodies were also assessed by the same method. Sites per cell with these antibodies were as follows: MAb BV-15 (11.7×10⁴ sites per cell); BV-12 (6.3×10⁴ sites per cell); and BV-06 (6.3×10⁴ sites per cell).

### EXAMPLE 3

### Antibody Specificity

Introduction: The specificity for tumor versus normal cells was measured for each of BV-15, BV-27, BV-12, BV-06, and comparative antibodies, 2C6 and BV-19. Four assessments of specificity were evaluated: i) Relative expression of OFA/iLRP on long-term cultured tumor and normal cells; ii) Binding to primary explants cultures of tumor and normal cells; iii) molecular evaluation of the antibody panel on Western blots and iv)immunohistology on fresh frozen and paraffin sections of tumor and normal tissues.

Cells and culture conditions: Three immortalized human myelogenous leukemia lines (K562, HL60 and NB4) and two human breast cancer cell lines (HCC1937 and HCC38 characterized as “triple negative” breast cancer) were examined by flow cytometry. In addition, normal epidermal keratinocytes and normal dermal fibroblasts were also examined here as were immortalized human epidermal keratinocytes (i.e., HaCaT) (Boukamp P, Petrussevska R T, Breitkreutz D, Hornung J, Markham A, Fusenig N E (1988) Normal keratinization in a spontaneously immortalized aneuploid human keratinocyte cell line. J Cell Biol 106(3):761-771.).

K562 cells: The first human immortalized myelogenous leukemia line, derived from a 53 year old female CML patient in blast crisis. The cells resembled both undifferentiated granulocytes and erythrocytes.

Culture conditions: The cells were cultured using RPMI-1640 (Life Technologies #21870), supplemented with 2 mM L-glutamine (Life Technologies #25030-081), 50 U penicillin/50 μg streptomycin (Life Technologies #15070-063), and 10% fetal bovine serum (Hyclone Laboratories, ATC31707). Cultures were maintained at 37° C. in a humidified atmosphere of 95% air and 5% CO₂. Culture doubling time was approximately 18 hours.

HL-60 cells: A human promyelocytic leukemia cell line, derived from a 36-year-old female with acute promyelocytic leukemia (principally a neutrophilic promyelocyte precursor). Spontaneous differentiation to mature granulocytes can be induced by dimethyl sulfoxide (DMSO), or retinoic acid. The compounds 1,25-dihydroxyvitamin (D₃), 12-O-tetradecanoylphorbol-13-acetate (TPA) and GM-CSF can induce HL-60 to differentiate to monocytic, macrophage-like and eosinophil phenotypes, respectively.

Culture conditions: Same as K562 cells, except 20% fetal bovine serum. Culture doubling time was about 24 hours.

NB4 cells: A human promyelocytic leukemia cell line very similar in characteristics to HL60. Culture conditions: Same as for HL60.

HCC38 cells (ATCC #CRL-2314): HCC38 is an epithelial cell line initiated from a primary, stage IIB, grade 3, ductal carcinoma with lymph node metastasis. HCC38 cell line was obtained from a patient with a prior history of leiomyosarcoma and whose mother died from breast cancer. HCC38 is positive for epithelial-specific cell marker, epithelial glycoprotein 2 (EGP2) and cytokeratin 19; and negative for the expressions of estrogen (ER) and progesterone (PR) receptors.

Culture conditions: The cells were cultured using RPMI-1640 (Life Technologies #21870), supplemented with 2 mM L-glutamine (Life Technologies #25030-081), 50 U penicillin/50 μg streptomycin (Life Technologies #15070-063), and 10% fetal bovine serum (Hyclone Laboratories, ATC31707). Cultures were maintained at 37° C. in a humidified atmosphere of 95% air and 5% CO₂.

HCC1937 cells (ATCC Catalog #CRL-2336): This cell line was initiated from a primary ductal carcinoma. The tumor was classified as TNM Stage IIB, grade 3. BRCA1 analysis revealed that the cell line is homozygous for the BRCA1 5382C mutation, whereas the lymphoblastoid cell line derived from the same patient is heterozygous for the same mutation. This mutation was present in two other family members; an identical sister also developed breast cancer. The cell line has an acquired mutation of TP53 with wild-type allele loss, an acquired homozygous deletion of the PTEN gene, and loss of heterozygosity at multiple loci known to be involved in the pathogenesis of breast cancer. The cells are negative for expression of Her2-neu and for expression of p53. HCC1937 is positive for the epithelial cell specific marker Epithelial Glycoprotein 2 (EGP2) and for cytokeratin 19. The cells are negative for expression of estrogen receptor (ER) and progesterone receptor (PR).

Culture conditions: same as HCC38.

Primary human dermal fibroblast: Human foreskin tissue was obtained from circumcisions performed at the University of Michigan Hospitals. De-identified tissue samples were obtained under an exemption from institutional review board oversight. This tissue was used to establish cultures of keratinocytes and dermal fibroblasts as described previously (Varani J, Perone P, Griffiths C E, Inman D R, Fligiel S E, Voorhees J J (1994) All-trans retinoic acid (RA) stimulates events in organ-cultured human skin that underlie repair. Adult skin from sun-protected and sun-exposed sites responds in an identical manner to RA while neonatal foreskin responds differently. J Clin Invest 94:1747-1756.).

Culture conditions: The cells were cultured in Dulbecco's Modified Minimal Essential Medium (Life Technologies #11960-044) supplemented with Nonessential Amino Acids (Life Technologies #11140-0 U penicillin/50 μg streptomycin (Life Technologies #15070-063), and 10% fetal bovine serum (Hyclone Laboratories, ATC31707). Cultures were maintained at 37° C. in a humidified atmosphere of 95% air and 5% CO₂.

Primary human epidermal keratinocytes: Keratinocytes were isolated and cultured from the same tissue as fibroblasts as described previously (Varani, supra). Cells were used at passage 2-3.

Culture conditions: Keratinocytes were cultured in Keratinocyte Growth Medium (KGM; Lonza; cc-3001) supplemented with gentamicin (Life Technologies #15750-060) (amphotericin-free). Cultures were maintained at 37° C. in a humidified atmosphere of 95% air and 5% CO₂.

Flow cytometry: Flow cytometric analysis was done using a BD LSRII cytometer (Serial #: H47300011; HTS Serial #: U30100321) and data were analyzed using BD FacsDiva Software. Briefly, cells in suspension were concentrated to 3-5×10⁵/ml, washed 2× in cold Standard Flow Buffer (SFB) and pelleted. Cells were then resuspended at 5×10⁵ in 100 μL of cold SFB in wells of a 96-well plate. The primary antibody was added to the cells and incubated for 30 minutes in the cold. After two wash steps, the fluorochrome-conjugated secondary was added and cells again incubated for 30 minutes. After two additional washes, the cells were fixed in 250 μl of 2% Formaldehyde solution per well. Cells were then assessed immediately or stored in the dark for up to 24 hours.

Confocal fluorescence microscopy: A Zeiss LSM 510 confocal microscope with a ×63 (C-Apochr) NA=1.2 water immersion objective lens was used for confocal microscopic analysis of substrate-attached cells. Laser excitation wavelengths included 364, 488, and 543 nm scanned in sequence by the line method. Briefly, cells were plated at 2×10⁴/well and cultured for 48 hours in LabTek 4-well chambered slides. After incubation, cells were washed gently 1× in warm DPBS (w/Ca²⁺Mg²⁺) and fixed with warm 2% formaldehyde for 20 minutes (500 μl/well). After three subsequent washes, cells were stored in PBS (w/Ca²⁺Mg²⁺) at 4° for up to 3 days. Cells were stained as follows: First, a blocking solution was applied for 90 minutes: The primary antibodies were then added in a 300 μl solution containing 1% BSA per well. Incubation was overnight at 4° C. The next day, the slides were washed three times (10 minutes each) and (in the dark), treated with fluorochrome-conjugated secondary antibody and phalloidin (to identify cytoskeletal structures). Incubation with the secondary antibody and phalloidin occurred concurrently for 60 minutes at room temperature. The slides were then washed three additional times and then cover-slipped with Prolong Gold (1 large drop per well) and visualized.

Western blotting (including preparation of cell lysates): Cells were harvested from culture, washed and then lysed in 1× cell lysis buffer consisting of 20 mM Tris-HCl (pH 7.4), 2 mM sodium vanadate, 1.0 mM sodium fluoride, 100 mM NaC1, 1% NP-40, 0.5% sodium deoxycholate, 25 μg/ml each of aprotinin, leupeptin and pepstatin, and 2 mM EDTA and EGTA. Lysis was performed by adding 200 μl of lysis buffer to each well and incubating the plate on ice for 5 minutes. After incubation, cells are scraped and samples sonicated. Then the extracts were cleared by microcentrifugation at 14000 g for 15 minutes. Western blotting with the various antibodies was carried out. Briefly, samples were separated in SDS-PAGE under denaturing and reducing conditions and transferred to nitrocellulose membranes. After blocking with a 5% nonfat milk solution in Tris-buffered saline with 0.1% Tween (TTBS) at 4° C. overnight, membranes were incubated for one hour at room temperature with the anti-OFA/iLRP antibodies diluted 1:2000 (typically) in 5% nonfat milk/TTBS. Thereafter, the membranes were washed with TTBS and bound antibody detected using the Phototope-HRP Western blot detection kit (Cell Signaling Technologies, Inc., Beverly, Mass.). Images were scanned, digitized and quantified. Prior to loading the gels, protein levels in each sample are determined using the BCA protein determination kit (Pierce Biotechnology, Rockford, Ill.) and equal amounts of protein were loaded onto each lane.

Immunohistology: Immunostaining was done using formalin-fixed, paraffin-imbedded tissue sections (individual cases and microarrays) and tissue sections prepared from OCT-frozen tissue. Biochain (San Francisco, Calif.) was the commercial vendor for the tissue arrays. Paraffin-sections were cut at 5 μm on a microtome and heated for 40 minutes at 65° C. Slides were then deparaffinized in xylene (3 changes for 2:00 minutes each). Slides were then rehydrated through graduated alcohols (100% alcohol, 95% alcohol, 70% alcohol, water) for 2 minutes each and finally submersed in tap water. Slides were then placed in TBS wash buffer until staining. Frozen Sections were cut from OCT-frozen tissue blocks and air dried for 30 minutes under a hood at room temperature, following which the sections were treated with cold 95% ethanol and acetone (50:50) for 10 minutes. After being air-dried for 30 minutes, slides were stained.

Slides prepared by either method were stained with the monoclonal antibodies of choice by the immunoperoxidase method. Briefly, slides were treated with “Background Sniper Block” (Biocare #BS966) for 10 minutes and then rinsed. The primary antibody was added for 30 minutes and the slides were then rinsed again. The secondary antibody (Mouse Secondary Reagent from Biocare #IPSC5001) was then applied for 10 minutes. Following three additional rinses, the Tertiary Reagent/HRP (Universal HRP Tertiary from Biocare #IPT5002) was applied for 10 minutes and the slides rinsed again. Chromagen (DAB) was added for 5 minutes and in some cases, the slides also received hematoxylin counterstain. After water rinse, the slides were dehydrated (70% alcohol, 95% alcohol, 100% alcohol; 2 minutes each step), exposed to three changes of xylene (2 minutes each) and coverslipped.

Evaluation was performed using an Olympus BX45 light microscope at total magnifications ranging from ×40 to ×600. The tumors and normal tissues were scored according to the intensity of staining on each sample from 1+ (weak) to 3+ (strong).

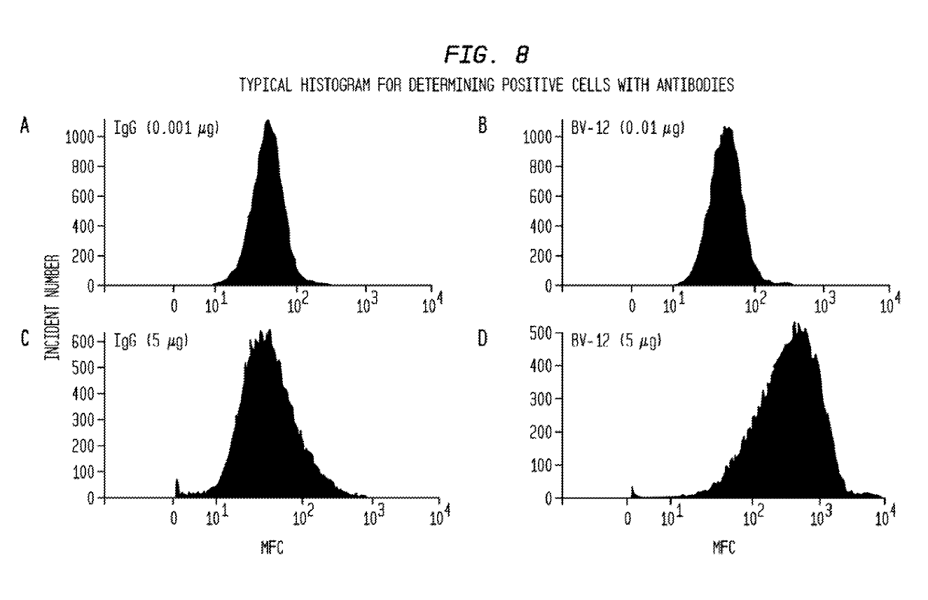

Results: Flow cytometry. Two acute myelogenous leukemia (AML) lines were examined. These included K562 cells (a prototypic undifferentiated human AML line) and HL60, an AML line differentiated to the promyeloblastic stage. A typical histogram from studies with the HL60 line and BV-12 is shown in FIGS. 8A-D.The data for all the antibodies is shown in Table 3 using HL-60 cells. (Values shown are the percentage of positive cells at each antibody concentration. Also shown are the mean fluorescence intensity values.) The binding pattern of antibodies to both cell lines is similar.

TABLE 3
Flow cytometricanalysis of anti-OFA MAb binding to
HL60 cells. Values shown are the percentage of positive cells
at each antibody concentration (expressed in units of μg).

| Antibody | 0.001 | 0.01 | 0.1 | 0.5 | 1.0 | 5.0 | 10 |
|---|---|---|---|---|---|---|---|
| IgG2a | 2.3 | 2.5 | 2.3 | 2.6 | 2.3 | 2.5 | 2.3 |
| IgG2b | 2.3 | 2.6 | 3.9 | 1.8 | 2.5 | 2.4 | 2.2 |
| BV-06 | 2.0 | 3.7 | 3.8 | 1.1 | 5.2 | 13.1 | 8.9 |
| BV-12 | 2.7 | 16.1 | 45.4 | 49.4 | 62.9 | 61.4 | 51.3 |
| BV-15 | 5.2 | 31.1 | 40.1 | 45.6 | 51.1 | 63.5 | 51.9 |
| BV-27 | 4.6 | 22.0 | 31.5 | 35.7 | 38.0 | 46.2 | 30.0 |
| BV-19 | 4.7 | 8.5 | 25.6 | 43.1 | 68.0 | 65.1 | 69.1 |

Western blotting: Cell surface staining can be used to identify differences between tumor cells and normal cells in antibody binding-and thereby establish specificity for tumor cells. However, specificity also relates to reactivity of the antibodies with 37 kD OFA/iLRP versus reactivity with the 67 kD mature laminin receptor protein. In order to compare binding to these two related protein species, Western blotting was utilized.

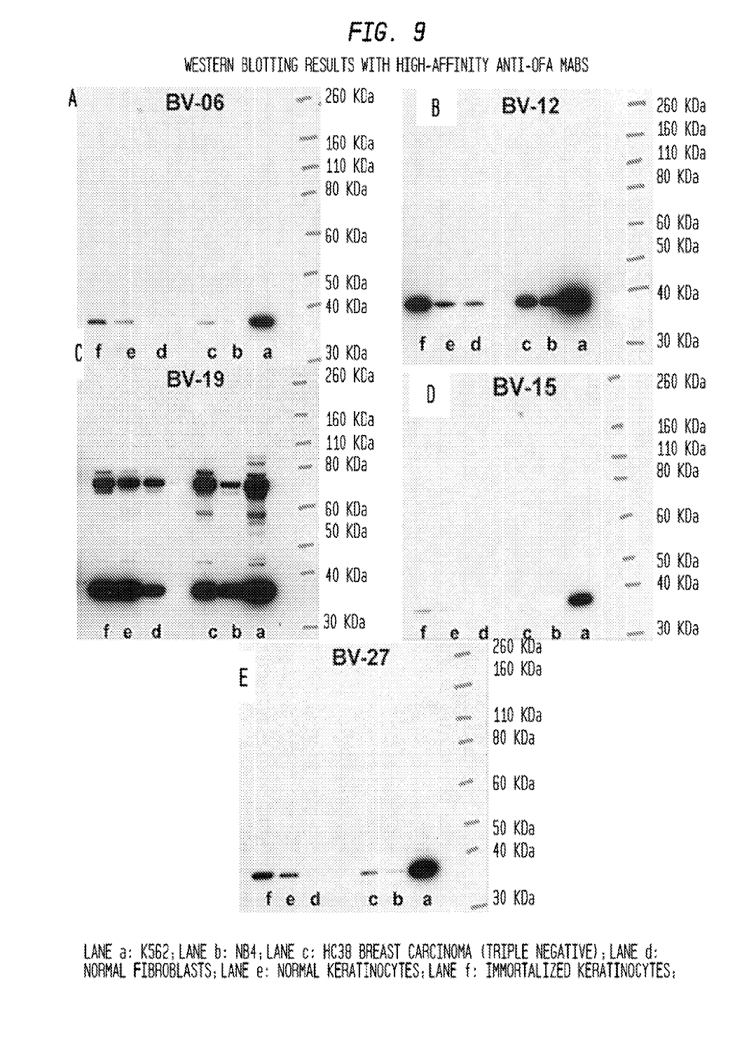

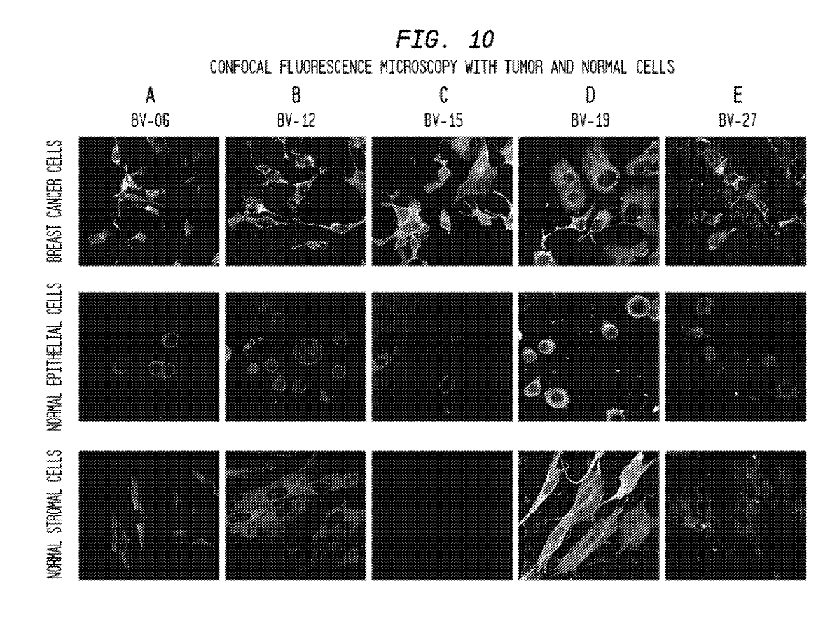

The data in FIGS. 9A-E show the results from the western blotting with each of the four antibodies of this invention as well as BV-19. Each panel a-f has a similar format. Specifically, lysates from three tumor lines (K562, NB4 and HCC38) are shown in lanes a-c. First passage fibroblasts and keratinocytes (normal human cells) are shown in lanes d and e. Lane f contains lysate from normal, immortalized human keratinocytes (HaCaT cells). Each lane contains 10 μg of cell lysate protein.

All of the antibodies of this invention reacted only with the 37 kD OFA molecule, whereas BV-19 in comparison also reacted with a 67 kD molecule, a hallmark of Mature Laminin Receptor. (See FIG. 9C.) BV-15 appeared to react only with 37 kD OFA present in tumor cells and only slightly with immortalized keratinocytes but not normal keratinocytes, a reactivity pattern expected for an antibody targeting an oncofetal antigen. (See FIG. 9D) The other antibodies of this invention had more reactivity to 37 kD OFA present in both tumor and normal cultured cells. (See FIGS. 9A, B, and E.)

Table 4 summarizes western blotting studies. Reactivity of K562 cell lysates with MAb BV-15 was arbitrarily set to 10 and reactivity with other lysates compared to this.

TABLE 4
Summary table of western blot data

| | Antibody: | | | | | |
|---|---|---|---|---|---|---|
| | | | | | BV-19 | |
| Cells | BV-15 | BV-06 | BV-27 | BV-12 | 37-Kd | 67-Kd |
| K562 | 10.0 | 5.0 | 8.0 | 9.0 | 9.0 | 7.0 |
| NB4 | 3.0 | <0.1 | 0.5 | 7.0 | 7.0 | 1.0 |
| Breast | 4.0 | 0.5 | 1.0 | 8.0 | 8.0 | 6.0 |
| Fibroblast | 1.0 | <0.1 | <0.1 | 2.0 | 1.5 | 2.0 |
| Keratinocyte | 1.5 | 0.5 | 1.0 | 4.0 | 5.0 | 3.0 |
| Immortalized | 4.5 | 1.0 | 3.0 | 8.0 | 5.0 | 3.5 |
| KC | | | | | | |

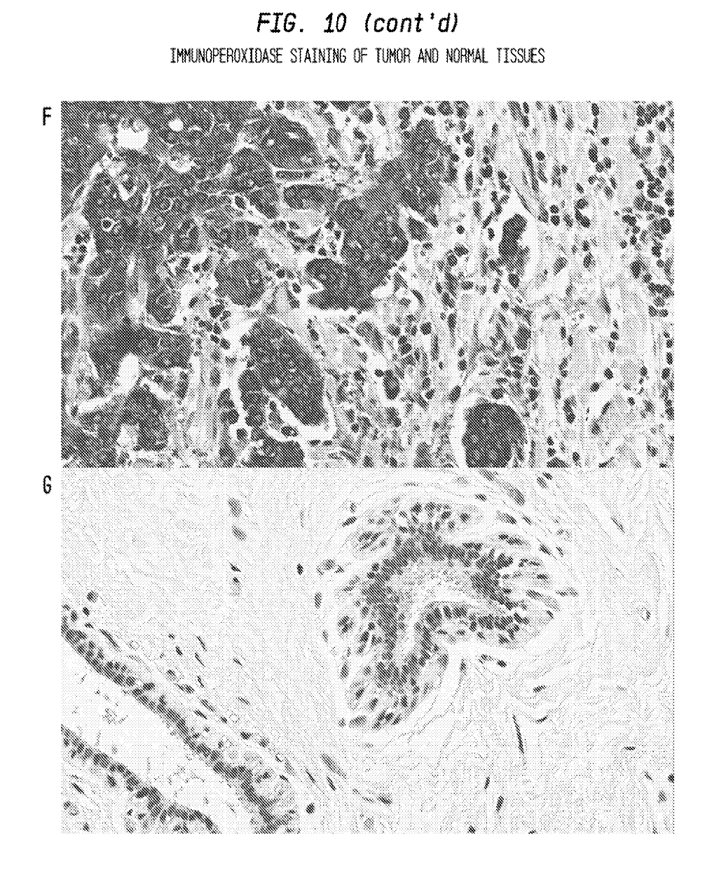

Confocal fluorescence microscopy: Confocal fluorescence microscopy was used to assess antibody binding to several different anchorage-dependent cell types. Among these were two triple-negative breast cancer cell lines (HCC38 and HCC1937) as well as primary cultures of human epidermal-derived keratinocytes and human dermal fibroblasts. Non-permeabilized cells were used in these assays so that cell surface antibody binding could be assessed. FIGS. 10A-E compare reactivity of the four inventive antibodies and BV-19 for comparison with one of the two breast cancer cell lines and with normal human epithelial cells and normal human fibroblasts. As shown, all five antibodies stained the breast cancer cells. With the four inventive antibodies, there was minimal staining of normal cellular components. (See FIGS. 10A-C, E.) The exception was the non-inventive MAb BV-19, which stained normal epithelial cells and fibroblasts as well as the breast cancer cells. (See FIG. 10D.)

Taken together, these findings from flow cytometry and confocal fluorescence microscopy are consistent with respect to tumor cell versus normal cell reactivity with antibodies to cell surface-associated 37 kD OFA/iLRP. Of interest, the 37 kD OFA is also an intracellular protein component of the ribosome (Ardini et al. Molec. Biol. Evol. 15:1017-1028 (1989)). As such, it is expressed in many normal cells. There was a correspondence in reactivity to the 67 kD form and reactivity to normal tissues as shown with BV-19. In contrast, the other antibodies with only reactivity to the 37 kD form expressed differential reactivity, at least at the cellular level, for tumor versus normal cultured cells.

Immunohistology: The above studies addressed comparisons among the inventive antibodies for reactivity with tumor cells versus normal cells in culture and for reactivity with 37 kD OFA/iLRP versus 67 kD mature LRP. In addition to these approaches, we used immunoperoxidase staining of tissue sections as an additional way to demonstrate tumor reactivity and to determine specificity between tumor tissue and normal tissue.

Monoclonal antibodies 2C6, BV-15 and BV-27 were examined against a battery of normal tissues in arrays. No binding was found in any major normal tissue for all of these antibodies reactive to the C-terminal portion of OFA. In contrast, BV-19, reactive to the Mature Laminin Receptor, demonstrated multiple normal cell reactivities including cells of epithelial and mesenchymal origin. Breast tumor tissue was examined with two of the antibodies of this invention showing the most specificity by Western blotting and confocal microscopy-BV-15, and BV-27. After initial titration of the three MAbs to identify optimal staining with each MAb, the one that gave the best staining of tumor tissue and the least background (i.e., MAb BV-15 at 1:40,000 dilution) was used in additional studies. OCT-frozen tissue from three breast cancer specimens and two age-matched controls were compared initially. Following this, 5 formalin-fixed breast cancer specimens were stained. There was more intense staining (overall) in tissue sections from breast cancer specimens than was seen in the normal controls. Representative images are shown in FIGS. 10F-G. Normal ducts were negative with the exception of slight, cytoplasmic staining for some ducts but not all.

Finally, a series of 5 formalin-fixed bone marrow biopsies were examined with MAb BV-15. Strong reactivity was seen against AML whereas certain or normal progenitors were less reactive and others were completely negative (data not shown).

Overall Conclusion on Specificity: The data from all of these methods of study indicate that the antibodies of the present invention demonstrate selective reactivity, and discriminate tumor from normal tissues. By point of comparison, BV-19 raised to the same immunogen, rOFA, reacts to the mature, 67 kD form of the antigen and the immature 37 kD form and expresses no selectivity in its reactivity. This distinction is important because the 67 kD (or mature) form of OFA is expressed widely in normal tissues. It has been reported that OFA is in normal mouse cortico-neurons and mouse liver. Unexpectedly, the antibodies of the present invention did not express reactivity to mouse liver but did react to certain brain neurons as demonstrated by immunoperoxidase staining of tissue. Thus the epitopes recognized by the antibodies of this invention are expressed more selectively than that reported for OFA antigen.

### EXAMPLE 4

### Determining Biological and Anti-Tumor Activity

A series of studies was conducted in vitro and then in in vivo animal models to evaluate the ability of inventive antibodies to block tumor growth. Since OFA/iLRP is a laminin-binding protein (1-4), one of the key questions addressed was whether the inventive monoclonal antibodies to OFA/iLRP would interfere with tumor cell-laminin interactions.

Monoclonal antibodies. The antibodies included in the in vitro studies included the antibodies of this invention

Cells: For these studies we utilized two anchorage-dependent murine fibrosarcoma lines that are known to express surface OFA/iLRP. These include the MCA-1315 line described by Coggins et al. (5) and the NP line established in our laboratory (1,4). In addition, we used a murine B-cell leukemia line (A20) which has also been shown to express surface OFW/iLRP (6).

Laminin: Murine laminin obtained commercially was used in these studies. Laminin was diluted in PBS and wells of a 24-well dish were coated with 0.1-0.5 μg of the purified protein per well (2 cm² in surface area). Coating was done by incubating the plates with the protein overnight at 37° C. in an atmosphere of 95% air/5% CO₂. Fibronectin served as a control. When fibronectin was used, it was coated onto wells of a 24-well dish exactly as was done with laminin except that 1 μg/well was used.

Cell attachment to laminin: MCA-1315 cells were harvested from monolayer culture and washed two times in PBS. Cells (5×10⁴ cells per well) were added to laminin-coated wells of a 24-well culture dish in culture medium consisting of serum-free DMEM medium with 200 μg/ml of bovine serum albumin (DMEM-BSA). Cells were incubated at 37° C. in an atmosphere of 95% air/5% CO₂. Fibronectin-coated wells served as control. At various times later, the non-attached cells were harvested and counted. Wells (with attached cells present) were flooded with 10% buffered formalin. Cell spreading was determined by counting the percentage of attached cells that had developed a spindle-shaped appearance. Formalin-fixed cells were then used for microscopy.

Proliferation: MCA-1315 cells (5×10⁴ cells per well) were harvested from culture, washed two times and plated in wells of a 24-well dish. Growth medium (DMEM medium containing 10% fetal bovine serum [DMEM-FBS]) was used for this. After attachment and spreading, the cells were washed and incubated in serum-free medium (DMEM-BSA) and treated with the monoclonal antibodies to OFA/iLRP at 25 μg/well. Two days later, cells were harvested and counted.

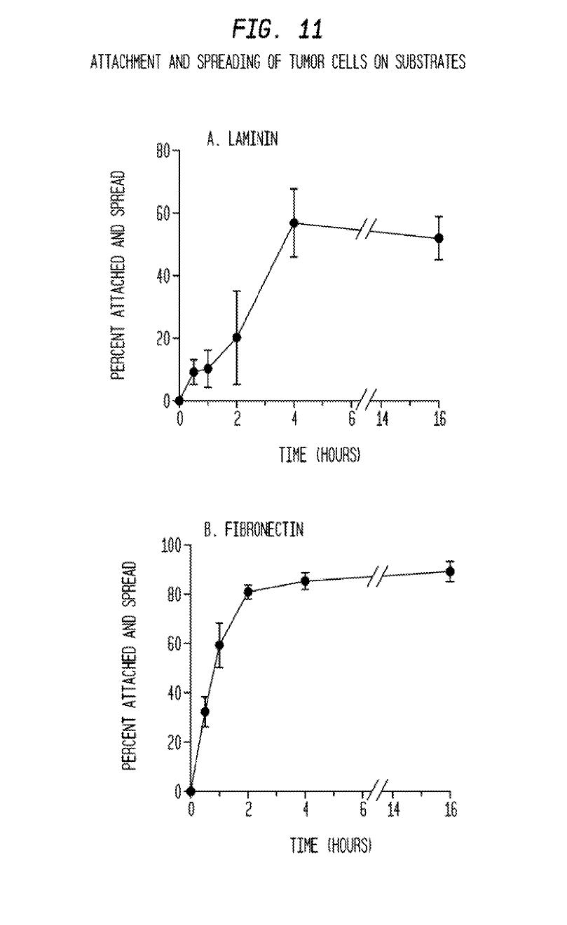

Results: Cell attachment to laminin. FIG. 11A demonstrates attachment of one of the two fibrosarcoma lines to laminin. The cells attached to laminin-coated wells under serum-free conditions (i.e., in DMEM-BSA). Attachment and spreading were time-dependent. Attachment and spreading occurred more rapidly and to a greater extent on fibronectin (FIG. 11B).

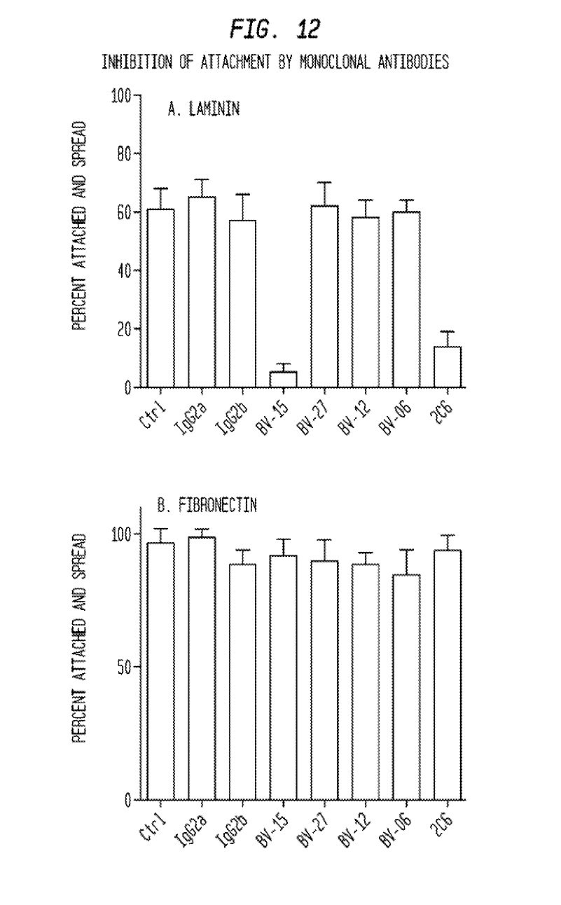

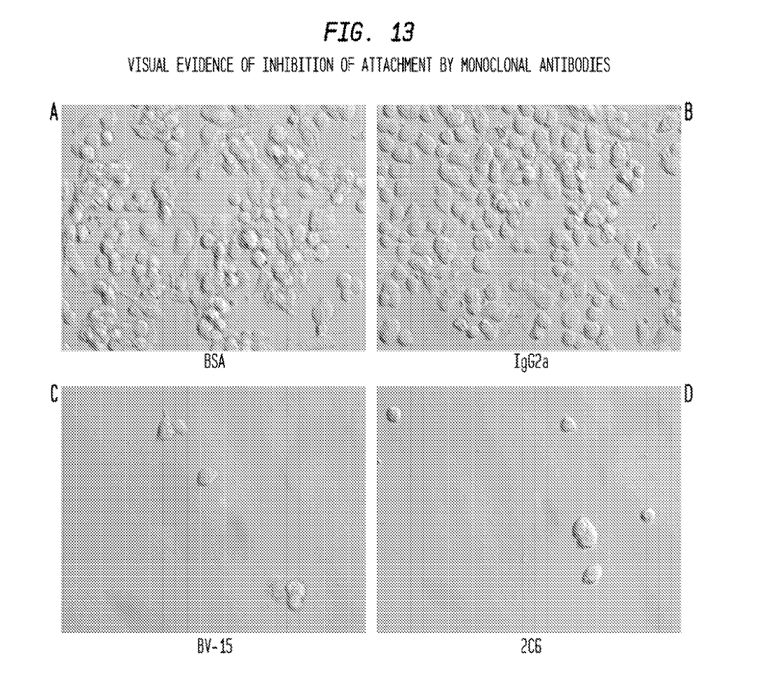

Using 0.5 ug of laminin per well and antibody concentrations of 25 ug/well, we determined the capacity of each antibody to interfere with attachment and spreading. Both BV-15 and MAb 2C6 inhibited cell attachment and spreading on the laminin substrate whereas BV-27 did not. Neither of the control IgGs or the other antibodies interfered with cell attachment and spreading on laminin. OFA contains a laminin binding site in the C-terminal portion but does not bind fibronectin. FIGS. 12A-B show that, as expected, the antibodies to OFA only inhibited attachment of cells to laminin. FIGS. 13A-D show microscopic evidence that MCA-1315 cells attach to laminin in the presence of control IgG2a (FIG. 13B) but not in the presence of either of the two anti-OFA/iLRP antibodies (FIGS. 13C and D). A second OFA/iLRP-positive murine fibrosarcoma cell line (NP) gave similar results to those presented with MCA-1315 cells (data not shown).

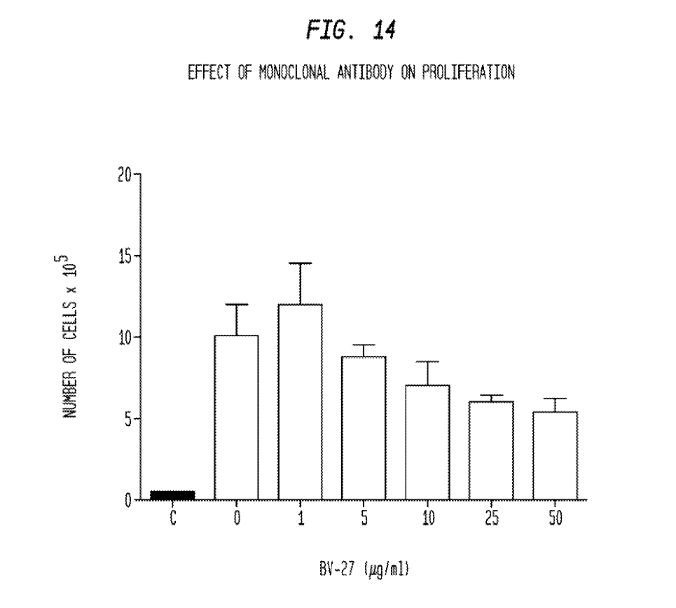

Proliferation: FIG. 14 shows results from studies in which monoclonal antibodies to OFA/iLRP were assessed for ability to interfere with proliferation. For these experiments, the A20 line of murine leukemia cells was used. Unexpectedly, only BV-27 inhibited proliferation while the antibodies (BV-15 and 2C6) that could alter attachment did not inhibit proliferation.

Conclusion: The data show that antibodies specific to OFA/iLRP suppressed attachment to laminin but had no effect on attachment to fibronectin. These findings are of interest because cell-substrate adhesive interactions are critical to all aspects of tumor biology and tumor metastases. If cell-substrate adhesive interactions are disrupted, subsequent events like metastases can be altered (7).

The data also demonstrate that certain of the monoclonal antibodies interfered with cell proliferation. It is of interest that monoclonal antibody BV-27, which did not have a major effect on cell attachment to laminin, was effective in suppressing proliferation. Direct inhibition of proliferation may predict suppression of primary tumor growth in vivo.

### REFERENCES

1. Malinoff, H. L., Wicha, M. S. Isolation of a cell surface receptor protein for laminin from murine fibrosarcoma cells. J. Cell. Biol. 1983. 96(5):1475-1479.

2. Rao, N. C. et al. Isolation of a tumor cell laminin receptor. Biochem. Biophys. Res. Commun. 1983. 111 (3) :804-808.

3. Viacavad, P. The spectrum of 67-kD laminin receptor expression in breast carcinoma progression. J. Pathol. 1997; 182(1):36-44.

4. Malinoff, H., Varani, J., McCoy, J. P., and Wicha, M. Metastatic potential of murine fibrosarcoma cells correlates with endogenous surface-receptor bound laminin. Int. J. Cancer, 1984; 33:651-655.

5. Rohrer J W, Barsoum A L, Coggin jr J H. Identification of oncofetal antigen—immature laminin receptor protein epitopes that activate BALB/c OFA/iLRP-specific effector and regulatory T-cell clones. J. Immunol. 2006: 176:2844-2856.

6. Coggin jr J H, Barsoum A L, Rohrer J W. 37-kD oncofetal antigen protein and immature laminin receptor protein are identical, universal T-cell inducing immunogens on primary rodent and human cancers. Anti-cancer Res. 1999: 19:5535-5542.

7. Aznavoorian S, Murphy A N, Stetler-Stevenson W G, Liotta L A. Molecular aspects of tumor cell invasion and metastasis. Cancer. 1993: 71:1368-1383.

### EXAMPLE 5

### Epitope Analysis and the Most Effective Binding Combination

A number of studies were carried out to assess potential interactions among individual antibodies to OFA/iLRP. Studies employing rOFA/iLRP were carried out by ELISA. Additional studies assessed antibody interactions with OFA/iLRP on intact cells. The initial purpose of these studies was to “map” cross-competition among the antibodies. Enhancement of MAb BV-15 binding to viable cells by co-incubation with MAb BV-27 was observed.

Monoclonal antibodies: The antibodies included the antibodies of this invention. Biotinylation was accomplished utilizing an EZ-Link® Sulfo-NHS-Biotinylation Kit from Thermo Scientific.

Assessment of MAb competition by direct ELISA. Antibody competition at 4° C. vs 24° C. was utilized to determine whether specific binding sites on the OFA molecule could be distinguished by the antibodies bound. Briefly, antigen (rOFA/iLRP) was coated onto the solid phase of micro-titer plates (Immulon IV, Nunc) in carbonate buffer at 0.5 μg/mL and allowed to bind overnight at 4° C. After washing and blocking with 0.5% BSA, antibody solutions of individual clones were added at 7 sequential Log₂ concentrations in diluent and allowed to react overnight at 4° C. The following morning the second plate was prepared as above. This plate was incubated for 1 hour at room temperature with the same concentrations of the individual antibodies. After the initial incubation period (of either length) and washing, a single concentration of a biotinylated antibody was added to both plates and they were allowed to react for 60 minutes at room temperature on a rotary platform shaker. After competitive antibody binding to OFA and subsequent streptavidin-HRP addition and incubation, bound biotinylated anti-OFA antibody was quantified utilizing TMB substrate addition and subsequent color development (Warner, et al., J. Agric. Food Chem. 34(4):714-717. DOI: 10.1021/jf00070a031.).

MAb binding to synthetic peptides: A series of 12-mer synthetic peptides covering the C-terminal region of the OFA/iLRP molecule was generated and analyzed for ability to serve as targets for the MAbs. Using the peptides as targets, the OFA/iLRP-epitope binding site for each anti-OFA/iLRP monoclonal antibody was identified, (Ball et al., J. Immunol. Meth. 171:37-44 (1994)). The peptide-based ELISA procedure involved using poly-L-lysine (PLL) as the anchor protein for the synthetic peptides, which were then covalently linked to the PLL using glutaraldehyde. The reactive aldehyde was then blocked by incubation in 1M glycine at room temperature. The plates were blocked for 2 hours with 200 μg/well of Casein Blocker in PBS. The avidin-Biotin system was used to develop the ELISA after treatment with biotinylated horse anti-mouse IgG.

Assessment of MAb cross-competition with intact cells: Biotinylated antibodies were also assessed for binding to HL-60 cells using flow-cytometry as described under specificity studies. For these studies, each of the other antibodies (non-biotinylated) was added along with the biotinylated “target” antibody. Fluorescence due to bound biotinylated antibody was then determined in the normal manner. Briefly, cells in suspension were concentrated to 3-5×10⁵/ml, washed 2× in cold Standard Flow Buffer (SFB) and pelleted. Cells were then resuspended at 5×10⁵ in 100 μL of cold SFB in wells of a 96-well plate. The primary (biotinylated) antibody was added to the cells and incubated for 30 minutes in the cold with or without the competitor, non-biotinylated antibody. After two wash steps, fluorochrome-conjugated streptavidin complex was added and cells again incubated for 30 minutes. After two additional washes, the cells were fixed in 250 μl of 2% Formaldehyde solution per well. Cells were then assessed immediately by flow cytometry or stored in the dark for up to 24 hours. Flow cytometric analysis was done using a BD LSRII cytometer (Serial #: H47300011; HTS Serial #: U30100321) and data were analyzed using BD FacsDiva Software. In some cases (as indicated in the Results Section), the amount of target antibody was varied.

Results from ELISA assays: For the initial experiments, each of the five antibodies (4 inventive antibodies and BV-19) were biotinylated and then unmodified versions all used as competitors. These initial studies were carried out at room temperature. There was a significant amount of cross-competition among the antibodies and it appeared that the degree of competition was due, in part at least, to antibody affinity. The studies were repeated but instead of adding the non-biotinylated “competitor” antibody for 1 hour at 24° C., the competitor antibody was added to the bound OFA/iLRP for 24 hours at 4° C. The rationale for this experiment was that if differences in antibody affinity were solely or largely responsible for what was observed, such differences could be reduced by the longer pre-incubation period. The standard protocol was run in parallel.

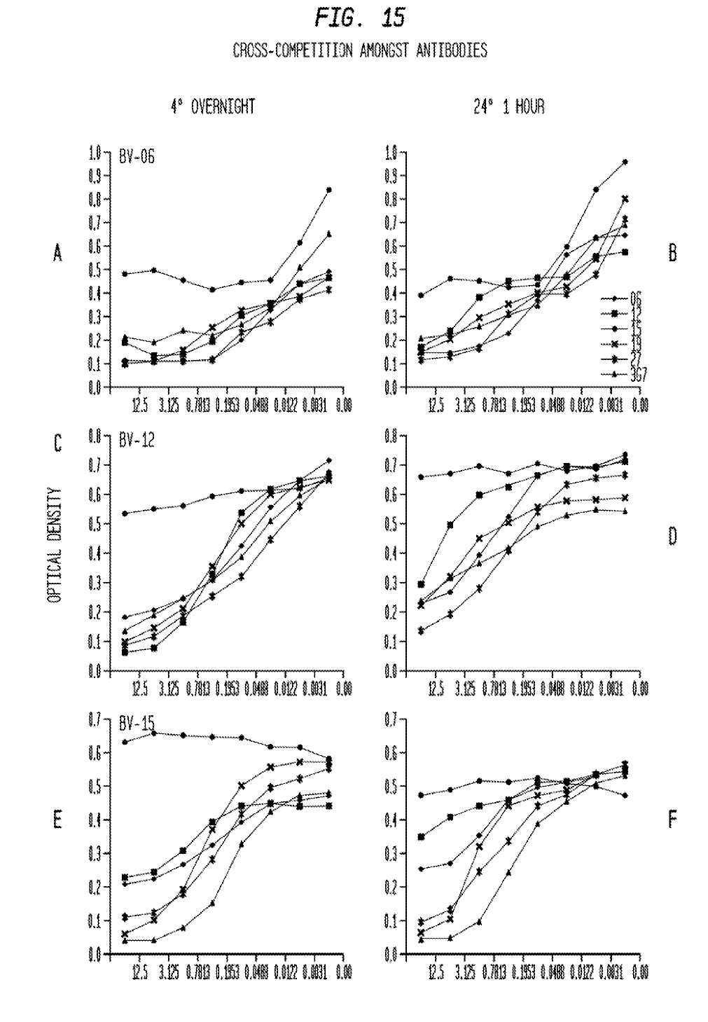

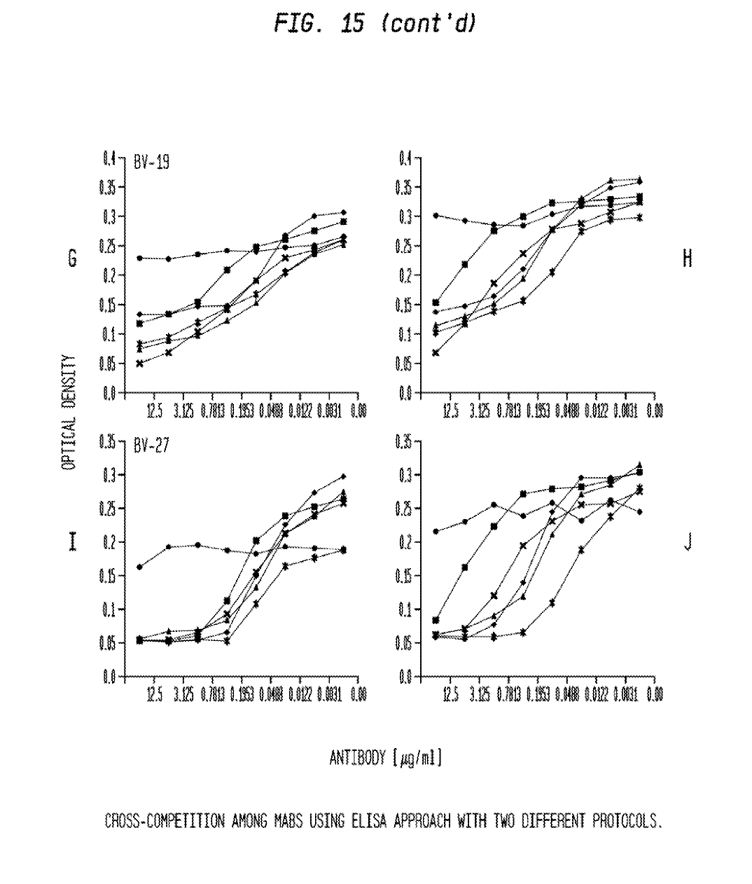

FIGS. 15A-J show results from the ELISA-based co-competition assay-comparing the two protocols (4° C. overnight on the left and 24° C. for 1 hour at right). In this comparative study, four inventive antibodies (FIGS. 15A, B, C, D, E, F, I, J) along with BV-19 (FIGS. 13G, H) were utilized as competitors. Two things are apparent from the figures. First, the 4° C. protocol did, in fact, reduce difference among the competitors. That is, the “tightness” of the data was closer with this protocol than was observed with the 24° C./1 hour protocol. Second, in spite of this, the data from the two protocols led to the same conclusion—i.e., that all four of the inventive antibodies competed to some degree with all the other MAbs. Neither 3G7 (a MAb prepared using OFA sequence of aa 131-144) nor mouse IgG competed with binding of any of the inventive antibodies (data not shown).

Table 5 summarizes the relative competitiveness of the antibodies with each of the biotinylated antibodies.

TABLE 5
Relative competition among the high-affinity MAbs to OFA.

| | Competitor MAb | | | | |
|---|---|---|---|---|---|
| Target MAb | BV-27 | BV-15 | BV-19 | BV-12 | BV-06 |
| BV-27 | BV-27 > | BV-15 > | BV-19 > | BV-06 > | BV-12 |
| BV-15 | BV-15 > | BV-27 = | BV-19 = | BV-06 > | BV-12 |
| BV-19 | BV-15 > | BV-27 = | BV-19 = | BV-06 > | BV-12 |
| BV-12 | BV-15 > | BV-27 = | BV-19 > | BV-06 > | BV-12 |
| BV-06 | BV-15 = | BV-19 = | BV-19 = | BV-06 = | BV-12 |

The target antibody was biotinylated and the competitor Mabs were not labeled.

Studies with synthetic peptides as target: Given the cross-reactivity observed in the direct ELISA and the suggestion that differences in antibody affinity were contributing to the results obtained, a second approach was used. A series of 12-mer peptides was made beginning at OFA/iLRP amino acid 177 and continuing with overlapping sequences (4 aa overlaps) to amino acid 295. The peptide sequences are shown in Table 6.

TABLE 6
Overlapping Peptide Sequences of OFA
Peptide

| | | |
|---|---|---|
| 1. | 177-188 | MLAREVLRMRGT |
| 2. | 181-192 | EVLRMRGTISRE |
| 3. | 185-196 | MRGTISREHPWE |
| 4. | 189-200 | ISREHPWEVMPD |
| 5. | 193-204 | HPWEVMPDLYFY |
| 6. | 197-208 | VMPDLYFYRDPE |
| 7. | 201-212 | LYFYRDPEEIEK |
| 8. | 205-216 | RDPEEIEKEEQA |
| 9. | 209-220 | EIEKEEQAAAEK |
| 10. | 213-224 | EEQAAAEKAVTK |
| 11. | 217-228 | AAEKAVTKEEFQ |
| 12. | 221-232 | AVTKEEFQGEWT |
| 13. | 225-236 | EEFQGEWTAPAP |
| 14. | 229-240 | GEWTAPAPEFTA |
| 15. | 233-244 | APAPEFTATQPE |
| 16. | 257-268 | VPIQQFPTEDWS |
| 17. | 261-272 | QFPTEDWSAQPA |
| 18. | 265-276 | EDWSAQPATEDW |
| 19. | 269-280 | AQPATEDWSAAP |
| 20. | 273-284 | TEDWSAAPTAQA |
| 21. | 277-288 | SAAPTAQATEWV |
| 22. | 281-292 | TAQATEWVGATT |
| 23. | 285-295 | TEWVGATTDWS |

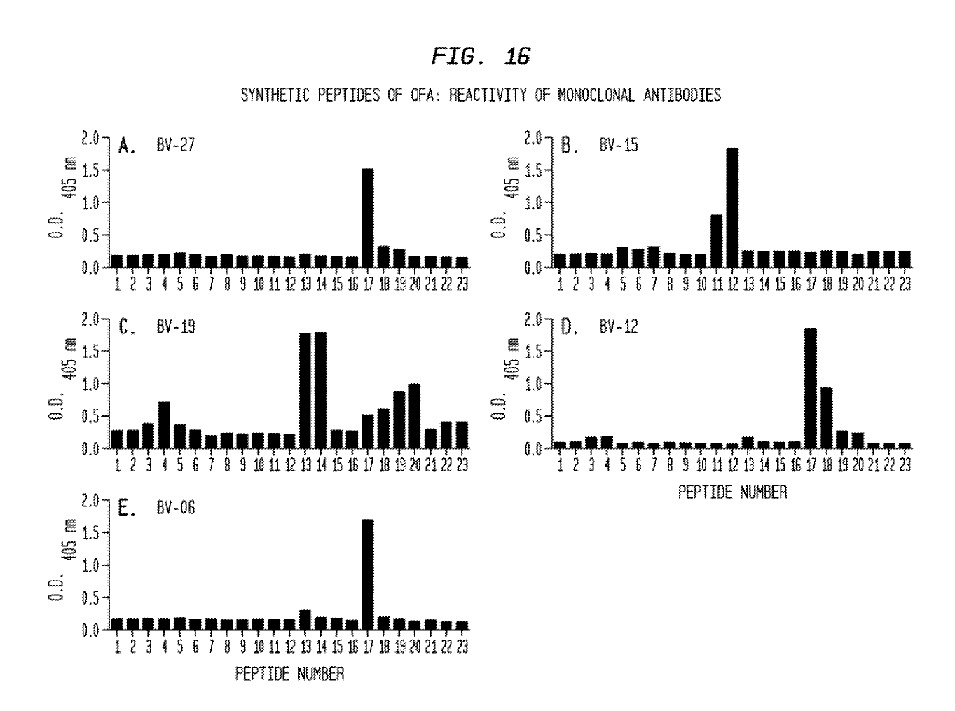

Using these peptides as target antigens in ELISA, each of the antibodies was mapped to one or more sequences. The data for the 4 inventive antibodies are shown in FIGS. 16A, B, D and E. Antibodies BV-06, and BV-27 mapped to peptide 17, amino acid residues 261-272 (QFPTEDWSAQPA) while BV-12 mapped to both peptides 17 and 18, amino acid residues 261-276 (QFPTEDWSAQPATEDW). (FIGS. 16B, D, and E). MAb BV-19 appears to bind to a discontinuous epitope that mapped primarily to peptides 13 and 14, amino acid residues 225-240 (EEFQGEWTAPAPEFTA) with reduced but detectable binding to peptide 4, amino acid residues 189-200 (ISREHPWEVMPD) as well as peptides 17-20, amino acid residues 261-284 (QFPTEDWSAQPA TEDWSAAPTAQA) (FIG. 16C). MAb BV-15 reacted with peptides 12 and 13, amino acid residues 217-232 (AAEKAVTKEEFQGEWT) (FIG. 16A). Although all the antibodies to the C-terminal portion of OFA demonstrated cross-inhibition of binding, they expressed unique reactivity patterns with the synthetic peptides. Most importantly, BV-19, which cross-reacts with the mature laminin receptor, recognizes a large, discontinuous epitope which contains distinct peptide segments from amino acid residue 189 through residue 284. This may explain the remarkable difference in specificity to OFA when compared to that of BV-27, BV-15, BV-06 and BV-12 (which do not cross-react with the mature protein).

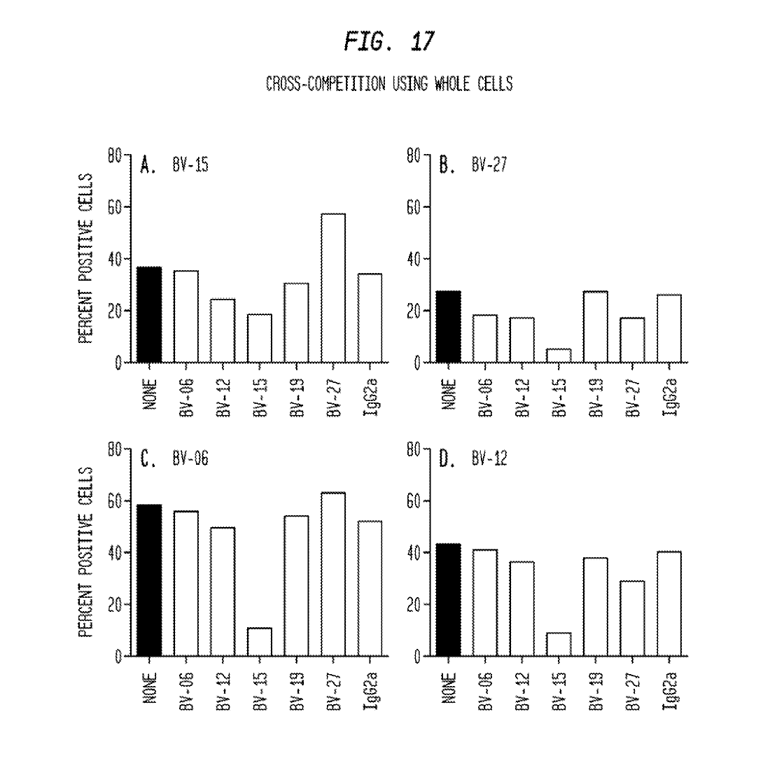

Results from intact cell assays: In a final set of experiments, the biotinylated antibodies and non-biotinylated antibodies were examined for cross-competition using intact HL-60 cells. After establishing concentrations for binding of the biotinylated antibodies, concentrations designed to obtain reactivity in approximately 50% of the cells (in all cases, this was 0.5 μg of the biotinylated MAbs). Each of the five competitor antibodies (non-labeled) were included at a single concentration—i.e., 10 μg/ml. The capacity of the competitor antibodies to reduce the percentage of positive cells was determined. The results are shown in FIGS. 17A-D. They can be summarized as follows.

First, non-biotinylated BV-15 effectively competed with itself and also competed with all of the other antibodies tested in this experiment (e.g., BV-27, BV-12, and BV-06) (FIG. 17B).

Second, non-labeled BV-27 effectively competed with biotinylated BV-27 (45% reduction) (FIG. 17A). It also competed with BV-12 (not surprising, in light of the peptide mapping studies) but did not compete with the other two antibodies utilized in this study. Unexpectedly, the presence of BV-27 in the mix enhanced binding with both antibodies (BV-06 and BV-15). Enhancement with BV-06 was modest, but the enhancement of BV-15 binding was strong. This finding is clearly unexpected from what would have been predicted based on the ELISA data and peptide mapping studies.

Third, IgG2a (as well as MAb BV-19) showed no inhibition with any of the antibodies (FIGS. 17A-D).

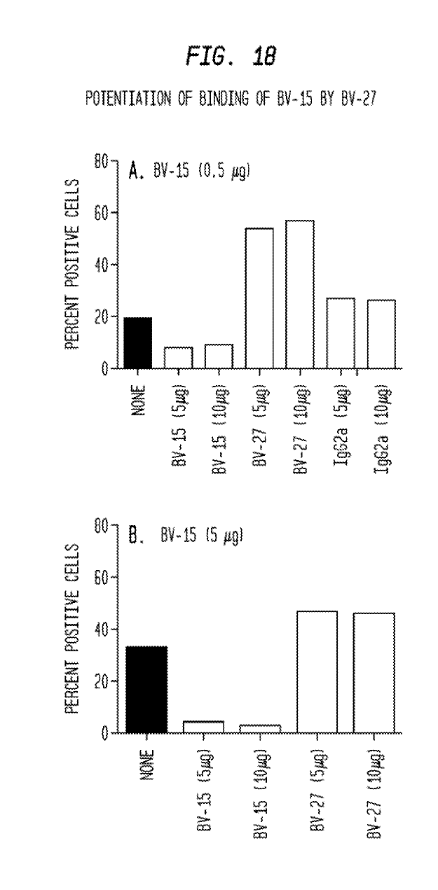

Given the unexpected enhancement of binding of MAb BV-15 by MAb BV-27, additional studies were carried out in which the biotinylated antibody (BV-15) was included at increasingly higher concentrations relative to the amount of MAb BV-27. The enhancement of binding of BV-15 by BV-27 was more apparent at low concentrations of the antibodies than at higher concentrations. (FIGS. 18A-B).

### EXAMPLE 6

### In Vivo, Therapeutic Evaluation

Monoclonal antibodies. The antibodies included in the analysis were BV-15 and BV-27.

Study design. The overall goal of these studies was to determine if anti-OFA/iLRP monoclonal antibodies could suppress primary tumor growth and/or metastasis formation by OFA/iLRP-positive cells in syngeneic mice. Two tumor cell lines were used. These include the murine B cell lymphoma line, A20, in Balb/c (syngeneic) mice and the B16 melanoma line in C57BL/6 (syngeneic) mice. Primary tumor growth and tumor formation following intravenous injection were assessed.

Primary tumor growth studies. Using a dose of tumor cells that will produce tumors in virtually 100% of the injected mice (5×10⁵ A20 cells per mouse or 1×10⁴ B16 melanoma cells), we carried out tumor growth inhibition studies with MAb BV-15 and BV-27. Mice were injected with tumor cells on day-0. Tumor bearing mice were injected with each of the potentially therapeutic antibodies or with the isotype matched control antibodies. One hundred (100) μg of antibody per mouse was injected on days 1, 4, 8, 11, 15, and 18 after tumor injection. The antibodies were injected via the intraperitoneal route. Tumor size was measured twice weekly and the animals were sacrificed on day-25(or earlier when signs of illness became apparent).

At the time of sacrifice, tumors were fixed in 10% buffered formalin and examined histologically. Blood was obtained from heart puncture at the time of sacrifice. In this protocol, 20 μl of blood was mixed with 2 ml of culture medium. Eight 10-fold dilutions were prepared and triplicate wells seeded with each dilution. The graded dilutions of the blood-culture medium were used so that the blood tumor load could be precisely determined.

At the time of sacrifice, lungs, liver and spleen were removed from animals and examined grossly and histologically for the presence of tumors. Grossly visible tumors were counted and sized.

IV tumor injection studies. For these studies, viable A20 tumor cells (5×10⁵) or B16 melanoma cells (1×10⁴) were introduced directly into the circulation by tail vein injection. Tumor bearing mice were injected with each of the potentially therapeutic antibodies or isotype matched control antibodies. We injected 100 μg of antibody per mouse. For the IV studies, mice were injected on 1 day before tumor injection and on days 1, 5, 8, 12 and 15. Animals were monitored daily for signs of illness and sacrificed on day-25 (or earlier when signs of illness became apparent, which occurred in two control mice).

Blood was obtained from heart puncture at the time of sacrifice and examined for tumor load as above.

At the time of sacrifice, lungs, liver and spleen were removed from animals and examined grossly and histologically for the presence of tumors.

Parameters assessed. In the IM tumor studies, primary tumor growth was assessed as a function of time. Histological features of the primary tumor in the control versus therapeutic antibody-treated animals were examined. Parameters assessed in the IV tumor studies included number of viable tumor cells in the blood at the time of sacrifice and number and size of lung and liver tumors. Tumor sections from both the primary tumors and the metastatic tumors were assessed for “activated caspase-3” staining as a measure of tumor apoptosis.

Statistical evaluation and group size. Fifteen animals per group were used in both the IM and the IV studies with A20. In B16 melanoma studies, there were 9-10 animals per group. Each parameter was analyzed separately using the unpaired T-test or one-way ANOVA followed by paired group comparison.

Results: The majority of the in vivo data (to date) has been generated using A20 cells as target. A primary tumor growth model entailed injecting the cells IM at a dose designed to induce tumors in <95% of the mice. Treatment was begun 1 day post tumor injection. In this model, we suppressed primary tumor growth.

A “metastatic” tumor model was also used with this tumor line. Mice were injected with tumor cells by the IV route. Treatment was begun 1 day prior to tumor cell injection and with additional antibody treatment on days 1, 5, 8, 12, and 15 days after tumor cell injection. In this model, a reduction in liver metastases, lung metastases and a reduction in blood borne tumor load were measured.

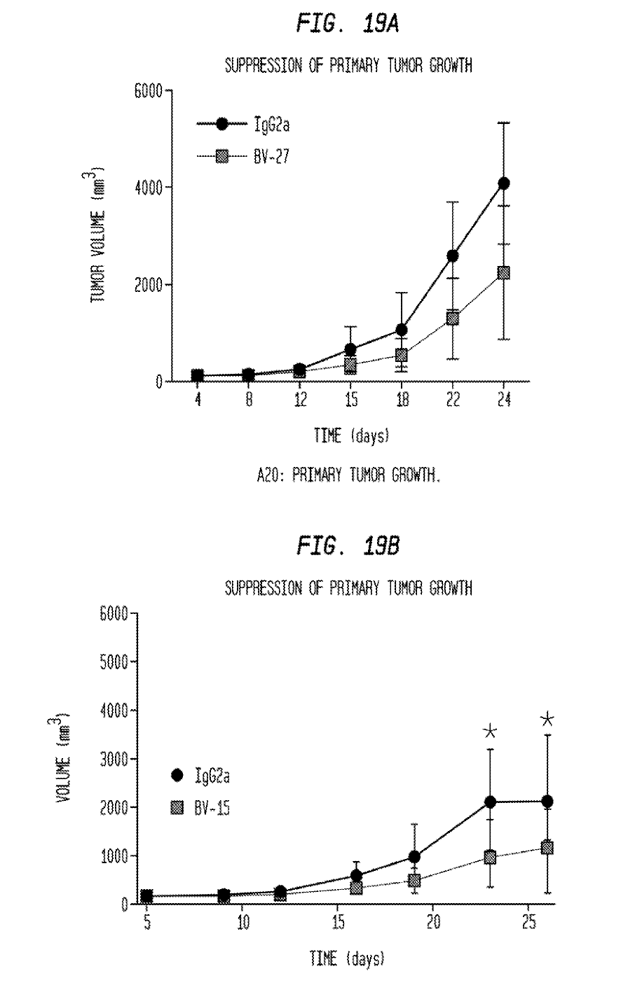

Results for BV-27 are shown in FIG. 19A. The antibody suppressed primary tumor growth (A20 cells) by about one-third. BV-15 in the same model also significantly suppressed tumor growth but less than BV-27 (FIG. 19B).

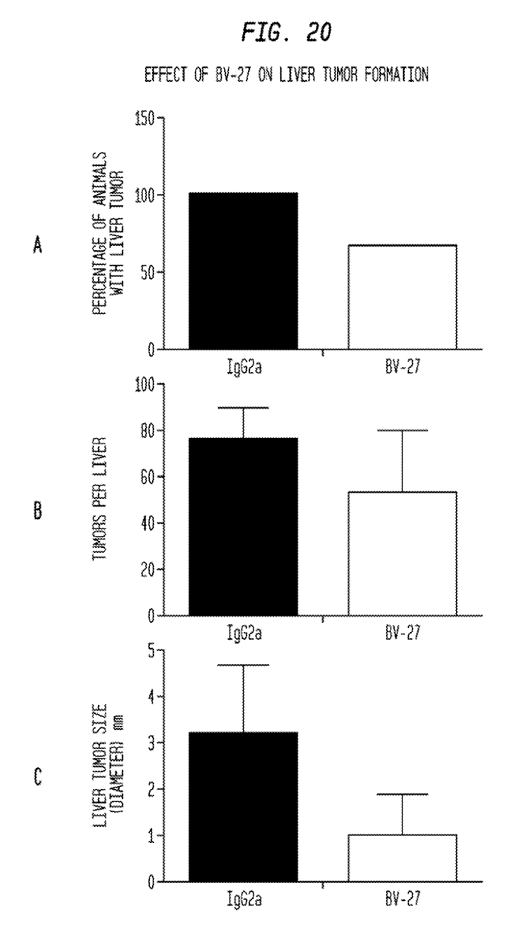

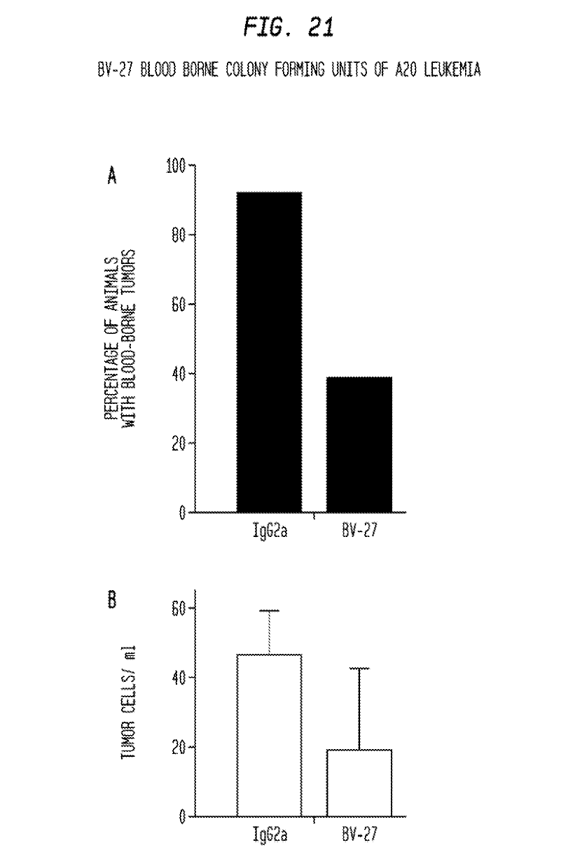

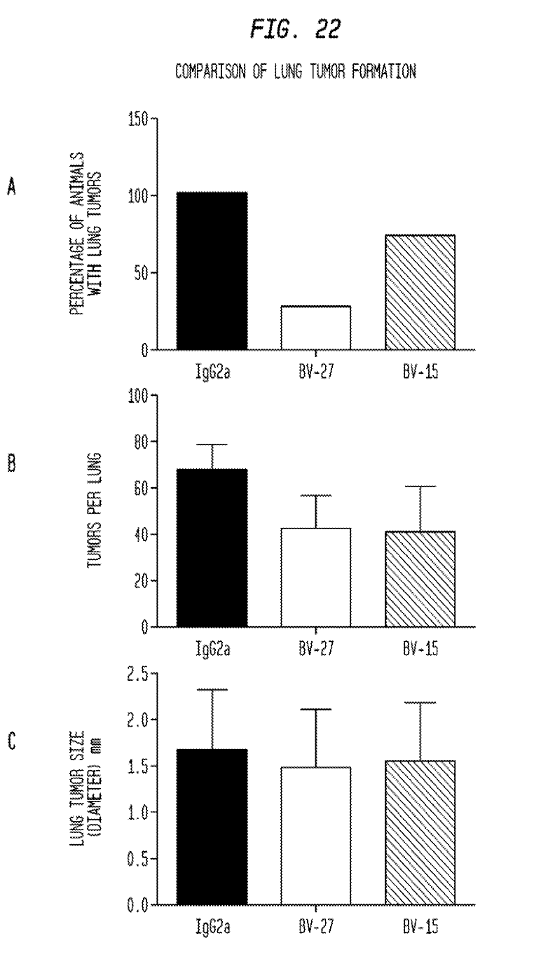

In the metastatic model, FIGS. 20A-C, 21A, B, and 22A-C show that BV-27 (which was the most effective of the four antibodies at inhibiting cell proliferation in vitro) was active in suppressing tumor formation (metastases) in all compartments of the body (liver, blood, and lung). Most striking was liver and lung tumor formation where not only was there an impact on number of tumors formed but a reduction in size of tumors also. FIGS. 22A-C also provide comparative data with BV-15 indicating it was less effective than BV-27.

### EXAMPLE 7

### Determination of OFA in Circulation as a Tumor Diagnostic in Dogs

The inventive antibodies may also be used as a diagnostic tool such as to analyze levels of OFA in the circulation of animals in the absence of tumor cells. This diagnostic tool measures circulating OFA and can be used to differentiate various stages of cancer in animals such as dogs from those successfully in remission undergoing treatment and animals with other conditions such as inflammation. Representative studies in dogs are described below.

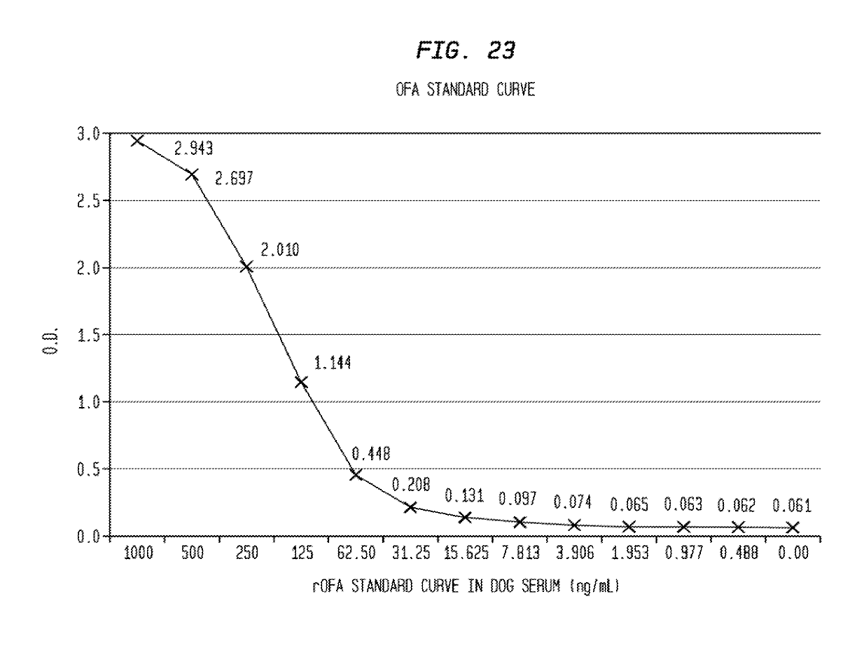

A sandwich ELISA was developed to measure the OFA levels in serum. Anti-OFA BV-27 antibody was coated at 1 μg/ml in Carbonate Buffer (pH 9.6) at 50 μL/well of anti-OFA. The plate was covered with an acetate sealer and incubated at 4° C. overnight. Prior to use the plate was washed 3 times with 200 μλ/well of DPBS-tween 20 (0.05%; DPBS-t) and 250 μλ/well of Blocking Buffer (0.5% BSA in 1× DPBS) was added and allowed to incubate on a platform shaker for 30 minutes at room temperature (R.T.). Samples and target antigen (rOFA) were diluted in Diluent Buffer, comprised of Superblock (Thermo-Fisher, Pierce #37515) diluted to 50/50 v/v with 1× DPBS. Serum from dogs with various stages of diagnosed cancer and various other diseases was diluted 1:25 in diluent. A standard curve of rOFA was made using Normal Healthy dog sera diluted 1:25 in Diluent Buffer, in a serial dilution (Log₂) from 1,000 ng/ml to 0.488 ng/ml and zero control. Plates were washed 3 times with 200 μL/well of DPBS-t and 50 μl/well of OFA Standards or Samples were added to each well and plates loosely covered with aluminum foil and incubated on a platform shaker for 60 minutes at R.T. At the end of the incubation period plates were washed 4×s w/DPBS-t and 50 μL/well of biotinylated anti-OFA antibody (BV-15 or BV-19) diluted 1:1,000 in diluent buffer was added to each well, loosely covered with aluminum foil and incubated on a platform shaker for 60 minutes at R.T. At the end of the incubation period plates were washed 5×s w/DPBS-t and 50 μL/well of Streptavidin-HRP (R & D Systems, #890803) diluted 1:400 in Diluent Buffer was added to each well, loosely covered with aluminum foil and incubated on a platform shaker for 30 minutes at R.T. At the end of the incubation period plates were washed 5×s w/DPBS-t and 100 μL/well of TMB Substrate (Thermo Fisher #34028) and color allowed to develop for up to 10 minutes. The reaction was stopped by adding 50 μL/well of H₂SO₄ (2 N) and plates read at 450 nM on ELISA Plate Reader. Computer software was used to plot the Log concentration of the rOFA standards on the x-axis and optical density on the y-axis generating a curve (FIG. 23) with a linear dose-response range of 62.5-500 ng/mL OFA. An equation of the line was generated by the software and used to determine the concentration of OFA in the dog serum.

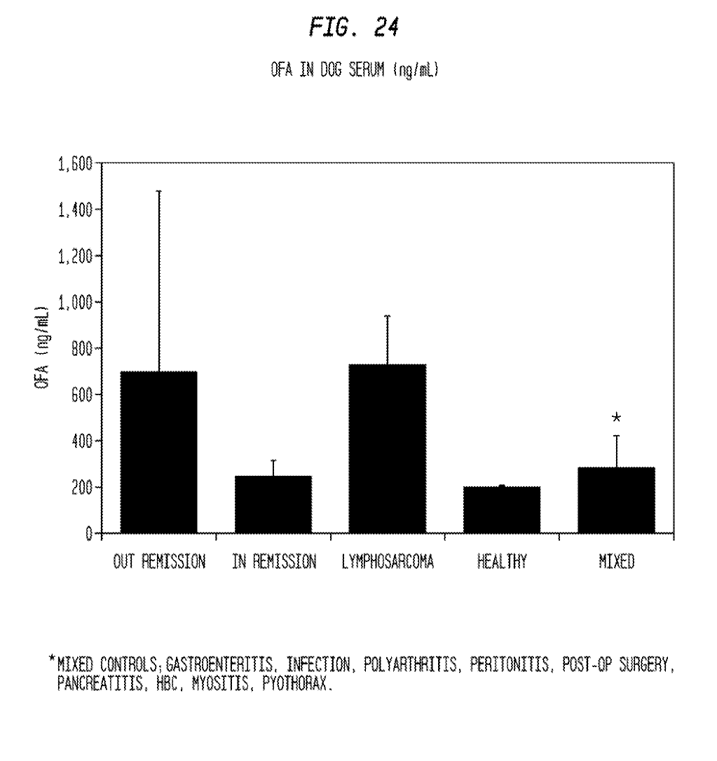

Test groups of animals in the original data set included dogs with lymphosarcoma as compared to animals in remission, healthy controls and control animals that had various inflammatory conditions. As shown in Table 7 there was a marked degree of difference in the detectable levels of OFA in serum of the dogs in the various groups. In the healthy control animals and in those animals with inflammatory conditions (mixed controls), the amount of OFA in the serum averaged 195.86+/−1.19 ng/ml for the healthy animals and 270.71+/−143.89 ng/ml for the inflammatory control animals. By comparison, those animals with active acute lymphosarcoma at the time of diagnosis had much higher levels of OFA in circulation (723.80+/−215.73 ng/ml), as well as animals which were in treatment but “out of remission” from their cancer (699.59+/−782.98 ng/ml) as compared to the control animals. Animals that were “in remission” of their cancer had lower levels of OFA (243.05+/−65.44 ng/ml) similar to the levels seen in the controls. These results are also shown in bar graph form (FIG. 24) and demonstrate the ability of the assay to detect circulating OFA in the blood. Secondly and most importantly it is also possible to detect different levels of OFA in the serum with high levels seen in the blood of animals with active tumors as compared to those in remission and control animals. Thus these data demonstrate that the measurement of circulating OFA is a useful diagnostic marker for the presence of cancers and for monitoring the success of therapy and reoccurrence of disease.

TABLE 7
Mean OFA in Dog Serum in ng/ml (range)

| Out | In | | Healthy | Mixed |
|---|---|---|---|---|
| Remission | Remission | Lymphosarcoma | Controls | Controls* |
| (n = 19) | (n = 9) | (n = 6) | (n = 5) | (n = 11) |
| 699.59 | 243.05 | 723 .80 | 195.86 | 270.71 |
| (243.75- | (195.33- | (447.58- | (195.33- | (195.33- |
| 3,711.00) | 356.00) | 927.75) | 198.00) | 659.25) |

*Mixed Controls; Gastroenteritis, Infection, Polyarthritis, Peritonitis, Post-Op Surgery, Pancreatitis, HBC, Myositis, Pyothorax.

### Deposit of Biological Materials

Biological material has been deposited under the terms of the Budapest Treaty at American Type Culture Collection (ATCC), 10801 University Blvd., Manassas, Va. 20108, USA. The identification, designation numbers and date of deposits for each of the four hybridoma cell lines are set forth in the table below.

| IDENTIFICATION | ATCC | DATE OF |
|---|---|---|
| REFERENCE | DESIGNATION NO. | DEPOSIT |
| Hybridoma Cell Line BV-06 | PTA-120412 | Jun. 13, 2013 |
| Hybridoma Cell Line BV-12 | PTA-120415 | Jun. 13, 2013 |
| Hybridoma Cell Line BV-15 | PTA-120413 | Jun. 13, 2013 |
| Hybridoma Cell Line BV-27 | PTA-120414 | Jun. 13, 2013 |

The hybridoma cell lines have been deposited under conditions that assure that access thereto will be available during the pendency of this patent application to one determined by the Commissioner of Patents and Trademarks to be entitled thereto under 37 C.F.R. §1.14 and 35 U.S.C. §122. The deposits represent pure cultures. The deposits are available as required by foreign patent laws in countries wherein counterparts of the subject application or its progeny are filed. However, it should be understood that the availability of a deposit does not constitute a license to practice the subject invention in derogation of patent rights granted by governmental action.

### CITATIONS OF OTHER PUBLICATIONS REFERENCED HEREIN

Method of analysis of non-competitive enzyme immunoassays for antibody quantification. Beatty J D, Beatty B G, Vlahos W G, Hill L R. J Immunol Methods. 1987 Jun. 26; 100(1-2):161-72. PMID: 3298436 [PubMed—indexed for MEDLINE]

Measurement of monoclonal antibody affinity by non-competitive enzyme immunoassay. Beatty J D, Beatty B G, Vlahos W G. J Immunol Methods. 1987 Jun. 26; 100(1-2):173-9. PMID: 2439600 [PubMed-indexed for MEDLINE]

Engineered antibodies of IgG1/IgG3 mixed isotype with enhanced cytotoxic activities. Natsume A, In M, Takamura H, Nakagawa T, Shimizu Y, Kitajima K, Wakitani M, Ohta S, Satoh M, Shitara K, Niwa R. Cancer Res. 2008 May 15; 68(10):3863-72. doi: 10.1158/0008-5472.CAN-07-6297. PMID: 18483271 [PubMed-indexed for MEDLINE]

Replacing the complementarity-determining regions in a human antibody with those from a mouse. Jones P T, Dear PH, Foote J, Neuberger M S, Winter G. Nature. 1986 May 29-Jun. 4; 321(6069):522-5. PMID: 3713831 [PubMed—indexed for MEDLINE]

Reshaping human antibodies: grafting an antilysozyme activity. Verhoeyen M, Milstein C, Winter G. Science. 1988 Mar. 25; 239(4847):1534-6. PMID: 2451287 [PubMed—indexed for MEDLINE]

Reshaping human antibodies for therapy. Riechmann L, Clark M, Waldmann H, Winter G. Nature. 1988 Mar. 24; 332(6162):323-7. PMID: 3127726 [PubMed—indexed for MEDLINE]

Minimizing immunogenicity of the SDR-grafted humanized antibody CC49 by genetic manipulation of the framework residues. Gonzales N R, Padian E A, De Pascalis R, Schuck P, Schlom J, Kashmiri S V. Mol Immunol. 2003 October; 40(6):337-49. PMID: 14522015 [PubMed indexed for MEDLINE]

Developing a minimally immunogenic humanized antibody by SDR grafting. Kashmiri S V, De Pascalis R, Gonzales N R. Methods Mol Biol. 2004; 248:361-76. PMID: 14970508 [PubMed-indexed for MEDLINE]

A possible procedure for reducing the immunogenicity of antibody variable domains while preserving their ligand-binding properties. Padlan E A. Mol Immunol. 1991 April-May; 28(4-5):489-98. PMID: 1905784 [PubMed—indexed for MEDLINE]

Comparison of surface accessible residues in human and murine immunoglobulin Fv domains. Implication for humanization of murine antibodies. Pedersen J T, Henry A H, Searle S J, Guild BC, Roguska M, Rees A R. J Mol Biol. 1994 Jan. 21; 235(3):959-73. PMID: 7507176 [PubMed—indexed for MEDLINE]

Humanization of murine monoclonal antibodies through variable domain resurfacing. Roguska M A, Pedersen J T, Keddy C A, Henry A H, Searle S J, Lambert J M, Goldmacher V S, Bidttler W A, Rees A R, Guild B C. Proc Natl Acad Sci U S A. 1994 Feb. 1; 91(3):969-73. PMID: 8302875 [PubMed—indexed for MEDLINE]

Humanization of an anti-p185HER2 antibody for human cancer therapy. Carter P, Presta L, Gorman C M, Ridgway J B, Henner D, Wong W L, Rowland A M, Kotts C, Carver M E, Shepard H M. Proc Natl Acad Sci USA. 1992 May 15; 89(10):4285-9. PMID: 1350088 [PubMed-indexed for MEDLINE]

Humanization of an antibody directed against IgE. Presta L G, Lahr S J, Shields R L, Porter J P, Gorman C M, Fendly B M, Jardieu P M. J Immunol. 1993 Sep. 1; 151(5):2623-32.

Humanization of an anti-vascular endothelial growth factor monoclonal antibody for the therapy of solid tumors and other disorders. Presta L G, Chen H, O'Connor S J, Chisholm V, Meng Y G, Krummen L, Winkler M, Ferrara N. Cancer Res. 1997 Oct. 15; 57(20):4593-9.

Humanization of a recombinant monoclonal antibody to produce a therapeutic HER dimerization inhibitor, pertuzumab. Adams C W', Allison D E, Flagella K, Presta L, Clarke J, Dybdal N, McKeever K, Sliwkowski M X. Cancer Immunol Immunother. 2006 June; 55(6):717-27. Epub 2005 Sep. 3.

A humanized antibody that binds to the interleukin 2 receptor. Queen C, Schneider W P, Selick H E, Payne P W, Landolfi N F, Duncan J F, Avdalovic N M, Levitt M, Junghans R P, Waldmann T A. Proc Natl Acad Sci USA. 1989 December; 86(24):10029-33.

Humanization of a murine monoclonal antibody by simultaneous optimization of framework and CDR residues. Wu H, Nie Y, Huse W D, Watkins J D. J Mol Biol. 1999 Nov. 19; 294(1):151-62. PMID: 10556035 [PubMed—indexed for MEDLINE]

Antibody humanization by framework shuffling. Dall'Acqua W F, Damschroder M M, Zhang J, Woods R M, Widjaja L, Yu J, Wu H. Methods. 2005 May; 36(1):43-60. PMID: 15848074 [PubMed—indexed for MEDLINE]

Structural correspondences between mouse and human immunoglobulin VH genes. Application to the humanization of mouse monoclonal antibodies. Poul M A, Lefranc M P. Ann NY Acad Sci. 1995 Sep. 29; 764:359-61. No abstract available. PMID: 7486546 [PubMed indexed for MEDLINE]

Kabat Database and its applications: future directions. Johnson G, Wu T T. Nucleic Acids Res. 2001 Jan. 1; 29(1):205-6. PMID: 11125092 [PubMed—indexed for MEDLINE]

Use of human germline genes in a CDR homology-based approach to antibody humanization. Hwang W Y, Almagro J C, Buss T N, Tan P, Foote J. Methods. 2005 May; 36(1):35-42. PMID: 15848073 [PubMed-indexed for MEDLINE]

Germline humanization of a non-human primate antibody that neutralizes the anthrax toxin, by in vitro and in silico engineering. Pelat T, Bedouelle H, Rees A R, Crennell S J, Lefranc M P, Thullier P. J Mol Biol. 2008 Dec. 31; 384(5):1400-7. doi: 10.1016/j.jmb.2008.10.033. Epub 2008 Oct. 19. PMID: 18976662 [PubMed-indexed for MEDLINE]

Germline humanization of a murine Abeta antibody and crystal structure of the humanized recombinant Fab fragment. Robert R, Streltsov V A, Newman J, Pearce L A, Wark K L, Dolezal 0. Protein Sci. 2010 February; 19(2):299-308. doi: 10.1002/pro.312. PMID: 20014445 [PubMed-indexed for MEDLINE]

Development of an optimized expression system for the screening of antibody libraries displayed on the Escherichia coli surface. Daugherty P S, Olsen M J, Iverson B L, Georgiou G. Protein Eng. 1999 July; 12(7):613-21. PMID: 10436088 [PubMed-indexed for MEDLINE] Free Article

Yeast surface display for screening combinatorial polypeptide libraries. Boder E T, Wittrup K D. Nat Biotechnol. 1997 June; 15(6):553-7. PMID: 9181578 [PubMed—indexed for MEDLINE]

All patent publications and non-patent publications are indicative of the level of skill of those skilled in the art to which this invention pertains. All these publications are herein incorporated by reference to the same extent as if each individual publication were specifically and individually indicated as being incorporated by reference.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. An antibody or a single-chain or antigen-binding fragment thereof that specifically binds a C-terminal epitope of oncofetal antigen (OFA)/immature laminin protein (iLRP) present on a tumor/cancer cell and which does not substantially cross-react with mature OFA/LRP or with non-cancer cells, wherein the antibody comprises a) three heavy chain complementarity determining regions (CDRs) which have the sequences GYTFTSYNMH, YIYPGNGGTNYNQKFKG, and GGYYYGSSWELYFDY, and three light chain CDRs which have the sequences RSSQSIVHSNGNTYLE, KVSNRFS, and FQGSHVPPT; b) three heavy chain CDRs which have the sequences GFSLTAYGVN, MIWGNGDTDYNSALKS, and YGY, and three light chain CDRs which have the sequences KSSQSLLDSDGKTYLN, LVSKVDS, and WQGTHFPFT; c) three heavy chain CDRs which have the sequences GFTFSSYTMS, TISSGGTYTYYPDSVKG, and LRY, and three light chain CDRs which have the sequences KSGQSLLDSDGKTYLN, LVSKLDS, and WQGTHFPQT; or d) three heavy chain CDRs which have the sequences GFSLTSYDIS, VIWTGGGTNYNSAFMS, and SFVY, and three light chain CDRs which have the sequences RSSQSLVHSNGNTYLH, KVSNRFS, and SQSTHVPWT, or a variant of any of said CDRs.

2. The antibody of claim 1, which is a monoclonal antibody.

3. The antibody of claim 2, which is a murine monoclonal antibody.

4. The antibody of claim 1, which comprises three heavy chain complementarity determining regions (CDRs) which have the sequences GYTFTSYNMH, YIYPGNGGTNYNQKFKG, and GGYYYGSSWELYFDY, and three light chain CDRs which have the sequences RSSQSIVHSNGNTYLE, KVSNRFS, and FQGSHVPPT.

5. The antibody of claim 4, which has a light chain variable region and a heavy chain variable region having the sequences illustrated in FIG. 1.

6. The antibody of claim 5, produced by the hybridoma cell line having the ATCC accession no. PTA-120412.

7. The antibody of claim 1, which comprises three heavy chain CDRs which have the sequences GFSLTAYGVN, MIWGNGDTDYNSALKS, and YGY, and three light chain CDRs which have the sequences KSSQSLLDSDGKTYLN, LVSKVDS, and WQGTHFPFT.

8. The antibody of claim 7, which comprises a heavy chain variable region and a light chain variable region having the sequences illustrated in FIG. 2.

9. The antibody of claim 8, produced by the hybridoma cell line having the ATCC accession no. PTA-120415.

10. The antibody of claim 1, which comprises three heavy chain CDRs which have the sequences GFTFSSYTMS, TISSGGTYTYYPDSVKG, and LRY, and three light chain CDRs which have the sequences KSGQSLLDSDGKTYLN, LVSKLDS, and WQGTHFPQT.

11. The antibody of claim 10, which comprises a heavy chain variable region and a light chain variable region having the sequences illustrated in FIG. 3.

12. The antibody of claim 11, produced by the hybridoma cell line having the ATCC accession no. PTA-120413.

13. The antibody of claim 1, comprising three heavy chain CDRs which have the sequences GFSLTSYDIS, VIWTGGGTNYNSAFMS, and SFVY, and three light chain CDRs which have the sequences RSSQSLVHSNGNTYLH, KVSNRFS, and SQSTHVPWT.

14. The antibody of claim 13, which comprises a heavy chain variable region and a light chain variable region having the sequences illustrated in FIG. 4.

15. The antibody of claim 14, produced by the hybridoma cell line having the ATCC accession no. PTA-120414.

16. The antibody of claim 1, which binds at least one OFA/iLRP epitope selected from the group consisting of 217-232 (AAEKAVTKEEFQGEWT), 261-272 (QFPTEDWSAQPA) and 261-276 (QFPTEDWSAQPATEDW).

17. The antibody of claim 1, which is conjugated to a cytotoxic agent.

18. A pharmaceutical composition comprising the antibody of claim 1, and a pharmaceutically acceptable carrier.

19. A method of cancer treatment, comprising administering to a subject with cancer a therapeutically effective amount of the antibody of claim 1.

20. The method of claim 19, wherein the subject is a human.

21. The method of claim 19, wherein the cancer is a hematopoietic cancer.

22. The method of claim 21, wherein the hematopoietic cancer is leukemia.

23. The method of claim 21, wherein the hematopoietic cancer is lymphoma.

24. The method of claim 19, wherein the cancer is characterized by the presence of a solid tumor.

25. The method of claim 24, wherein the cancer is liver cancer.

26. The method of claim 24, wherein the cancer is lung cancer.

27. The method of claim 24, wherein the cancer is breast cancer.

28. The method of claim 24, wherein the cancer is colon cancer.

29. The method of claim 24, wherein the cancer is prostate cancer.

30. The method of claim 24, wherein the cancer is pancreatic cancer.

31. A method of diagnosing cancer in a subject, comprising contacting a tissue or fluid sample obtained from a subject with an antibody of claim 1, wherein the antibody is detectably labeled, and detecting complexation of the antibody and OFA/iLRP, wherein relative amount of OFA/iLRP in the sample relative to a control is indicative of a diagnosis of cancer in the subject.

32. The method of claim 31, wherein the detecting comprises an ELISA and wherein a fluid sample obtained from the subject is contacted with a capture antibody disposed on a solid support, and which is selected from the group consisting of BV-6, BV-12, BV-15 and BV-27, so as to allow formation of a complex between the OFA/iLRP and the capture antibody, followed by contacting with a non-cross inhibiting, detectably labeled detection antibody that binds OFA/iLRP and which is different from the capture antibody, detecting complexation of the detection antibody with the OFA/iLRP, and determining relative amount of the OFA/iLRP in the fluid sample relative to a control, wherein elevated amounts of OFA/iLRP in the fluid sample relative to the control is indicative of a diagnosis of cancer.

33. The method of claim 32, wherein the capture antibody is BV-27.

34. The method of claim 33, wherein the detection antibody is BV-15.

35. The method of claim 32, wherein the fluid sample is obtained from a human.

36. The method of claim 32, wherein the fluid sample is obtained from a non-human animal.

37. The method of claim 32, wherein the fluid sample is blood or serum.

38. The method of claim 32, wherein the non-human animal is a dog.

39. The method of claim 31, wherein the label is a chromogenic agent.

40. The method of claim 31, wherein the label is a fluorescent agent.

41. The method of claim 31, wherein the label is a chemiluminescent agent.
